(19) United States
(12) Patent Application Publication
(10) Pub. No.: US 2012/0315270 A1
(43) Pub. Date: Dec. 13, 2012

(54) RSV IMMUNOGENS, ANTIBODIES AND COMPOSITIONS THEREOF

(75) Inventors: Jason S. McLellan, Rockville, MD (US); Peter D. Kwong, Washington, DC (US); Barney S. Graham, Rockville, MD (US); William R. Schief, Encinitas, CA (US); Joe Jardine, Seattle, WA (US); Bruno Correia, San Diego, CA (US); Man Chen, Bethesda, MD (US)

(73) Assignees: THE UNITED STATES OF AMERICA, AS REPRESENTED BY THE; UNIVERSITY OF WASHINGTON, Seattle, WA (US); SECRETARY, DEPARTMENT OF HEALTH AND HUMAN SERVICES, Bethesda, MD (US)

(21) Appl. No.: 13/502,595

(22) PCT Filed: Oct. 21, 2010
(86) PCT No.: PCT/US2010/053558

§ 371 Date: Aug. 24, 2012

Related U.S. Application Data
(60) Provisional application No. 61/253,826, filed on Oct. 21, 2009.

(51) Int. Cl. A61K 39/12; A61K 39/42; A61P 31/14; C07K 16/10; C07K 14/08; C07H 21/04

(52) U.S. Cl. 424/133.1; 530/324; 536/23.72; 424/186.1; 530/387.3; 536/23.53

(57) ABSTRACT

The present invention provides immunogens that protect against RSV infection. The present invention also provides antibody proteins that protect against RSV infection. Such immunogens and antibody proteins are produced based on three-dimensional models also included in the invention. One model is of a complex between motavizumab and its antibody-binding domain on RSV fusion (F) protein. A second model is of a complex between 10 IF antibody and its antibody-binding domain on RSV F protein. The immunogens disclosed herein have been modified to elicit a humoral response against RSV F protein without eliciting a significant cell-mediated response against RSV. Such immunogens can comprise a scaffold into which RSV contact residues are embedded. The present invention also includes methods that utilize the disclosed three-dimensional models to produce immunogens and antibody proteins of the present invention. Also disclosed are methods of using the disclosed immunogens, for example to protect individuals from RSV infection. Also disclosed are methods of using the disclosed antibody proteins, for example to protect individuals from RSV infection.

This application claims the benefit of U.S. Provisional Patent Application No. 61/253,826, filed Oct. 21, 2009, which is hereby expressly incorporated by reference in its entirety.

### FIELD

The present invention relates to novel compositions that protect individuals from respiratory syncytial virus (RSV) infection. In particular, the present invention relates to vaccines that elicit antibodies having a high affinity for the RSV fusion (F) protein. The present invention also relates to therapeutic compositions comprising antibodies having a high affinity for the RSV F protein.

### BACKGROUND

Respiratory syncytial virus (RSV) is a highly contagious member of the Paramyxoviridae family of viruses that causes significant worldwide morbidity and mortality each year, particularly in infants. RSV infects people repeatedly throughout life, and causes significant morbidity in healthy children and adults. The RSV fusion (F) protein (see, e.g., Lopez J A et al., 1998, J. Virol. 72, 6922-6928) and antibodies thereto, have been targets for vaccine efforts. There is currently no licensed RSV vaccine. A previous vaccine trial in the 1960s containing a formalin-inactivated RSV actually enhanced the severity of disease upon natural infection with RSV. This was thought to have occurred due to an imbalanced T-cell response and elicitation of low avidity antibodies. Since there is currently no licensed RSV vaccine, passive immunization is used to prevent RSV infection, especially in those infants with prematurity, bronchopulmonary dysplasia, or congenital heart disease. Originally, RSV-neutralizing polyclonal antibodies from pooled human sera (RESPIGAM®) were used (see, e.g., Groothuis J R et al., 1995, Pediatrics 95, 463-467). This treatment was followed by the development of palivizumab (SYNAGIS®) (see, e.g., Johnson S et al., 1997, J. Infect. Dis. 176, 1215-1224). Palivizumab was humanized from mouse antibody 1129, which binds a 24-amino acid, linear, conformational epitope on the RSV F protein (see, e.g., Beeler J A, et al., 1989, J. Virol. 63, 2941-2950; Arbiza J et al., J. Gen. Virol. 73, 2225-2234, Lopez J A et al., 1993, J. Gen. Virol. 74, 2567-2577). Palivizumab binds to the F protein and thereby neutralizes the virus. Such treatments are expensive, costing approximately $1000 per dose. Moreover, the antibodies must be administered on a monthly basis during the winter months, thereby adding to the cost of treatment. When administered at a dose of 15 mg/kg each month during the RSV season, palivizumab reduces RSV-related hospitalizations by 55% (see, e.g., The Impact-RSV Study Group, 1998, Pediatrics 102, 531-537). Thus, a vaccine requiring only one or two administrations would have advantages over the current preventative treatment for RSV.

### SUMMARY

The present invention provides immunogens that protect against RSV infection. The present invention also provides antibody proteins that protect against RSV infection. Such immunogens and antibody proteins are produced based on three-dimensional models also included in the invention. One model is of a complex between motavizumab and its antibody-binding domain on RSV fusion (F) protein. A second model is of a complex between 101F antibody and its antibody-binding domain on RSV F protein. The immunogens disclosed herein have been modified to elicit a humoral response against RSV F protein without eliciting a significant cell-mediated response against RSV. Such immunogens can comprise a scaffold into which RSV contact residues are embedded. The present invention also includes methods that utilize the disclosed three-dimensional models to produce immunogens and antibody proteins of the present invention. Also disclosed are methods of using the disclosed immunogens, for example to protect individuals from RSV infection. Also disclosed are methods of using the disclosed antibody proteins, for example to protect individuals from RSV infection.

The present disclosure provides an RSV immunogen comprising an amino acid sequence of 1LP1_b (SEQ ID NO: 11) having from one to twenty amino acid substitutions, wherein at least one amino acid substitution is selected from the group consisting of: (a) substitution of a serine at amino acid position N-25 in SEQ ID NO:11; (b) substitution of a leucine at amino acid position 1-28 in SEQ ID NO:11; (c) substitution of a serine at amino acid position Q-29 in SEQ ID NO: 11; (d) substitution of an isoleucine at amino acid position L-31 in SEQ ID NO:11; (e) substitution of an asparagine at amino acid position K-32 in SEQ ID NO:11; (f) substitution of an aspartic acid at amino acid position K-4 in SEQ ID NO:11; (g) substitution of a lysine at amino acid position Q-6 in SEQ ID NO:11; (h) substitution of a lysine at amino acid position Q-7 in SEQ ID NO:11; (i) substitution of a leucine at amino acid position N-8 in SEQ ID NO:11; (j) substitution of a serine at amino acid position F-10 in SEQ ID NO:11; and (k) substitution of an asparagine at amino acid position Y-11 in SEQ ID NO:11. Such an RSV immunogen can comprise all of the substitutions (a) through (k). The present disclosure also provides an RSV immunogen comprising an amino acid sequence of truncated 1S2X_a (SEQ ID NO:13) having from one to twenty-five amino acid substitutions, wherein at least one amino acid substitution is selected from the group consisting of: (a) substitution of a serine at amino acid position 92 in SEQ ID NO:13; (b) substitution of a leucine at amino acid position 95 in SEQ ID NO:13; (c) substitution of a serine at amino acid position 96 in SEQ ID NO:13; (d) substitution of an isoleucine at amino acid position 98 in SEQ ID NO:13; (e) substitution of an asparagine at amino acid position 99 in SEQ ID NO:13; (f) substitution of an aspartic acid at amino acid position 100 in SEQ ID NO:13; (g) substitution of an asparagine at amino acid position 105 in SEQ ID NO:13; (h) substitution of an aspartic acid at amino acid position 106 in SEQ ID NO:13; (i) substitution of a lysine at amino acid position 108 in SEQ ID NO:13; (j) substitution of a lysine at amino acid position 109 in SEQ ID NO:13; (k) substitution of a leucine at amino acid position 110 in SEQ ID NO:13; (l) substitution of a serine at amino acid position 112 in SEQ ID NO:13; and (m) substitution of an asparagine at amino acid position 113 in SEQ ID NO:13. Such an RSV immunogen can comprise all of the substitutions (a) through (m). The present disclosure also provides a nucleic acid molecule comprising a nucleic acid sequence that encodes any of such RSV immunogens. Also provided is a recombinant molecule comprising such a nucleic acid molecule. Also provided is a recombinant cell comprising such a recombinant molecule. Also provided are uses of such nucleic acid molecules, recombinant molecules, and recombinant cells to produce any of such RSV immunogens. A recombinant molecule can also be a nucleic acid vaccine. Also provided is a composition that can include any of the RSV immunogens, nucleic acid molecules, recombinant molecules, or recombinant cells. Also provided is a vaccine comprising any of such RSV immunogens. The disclosure provides a method to elicit a neutralizing humoral immune response against RSV; such method comprises administering any of such RSV immunogens or vaccines, wherein such administration elicits a neutralizing humoral immune response against RSV. Also provided is a method to protect a patient from RSV infection; such method comprises administering to the patient any of such RSV immunogens or vaccines, wherein such administration protects the patient from RSV infection.

The disclosure provides an immunogen comprising an antibody-binding domain that binds an antibody selected from the group consisting of motavizumab and 101F antibody, wherein the three-dimensional structure of the antibody-binding domain of the immunogen spatially corresponds to a three-dimensional structure of an antibody-binding domain of a fusion (F) peptide derived from respiratory syncytial virus (RSV) fusion (F) protein in a complex selected from the group consisting of:

(a) a complex between a F peptide consisting of amino acid sequence SEQ ID NO:2 and motavizumab, the complex being set forth in the three-dimensional model defined by the atomic coordinates specified in Research Collaboratory for Structural Bioinformatics (RCSB) Protein Data Bank accession code 3IXT; and

(b) a complex between a F peptide consisting of amino acid sequence SEQ ID NO:4 and 101F antibody, the complex being set forth in the three-dimensional model defined by the atomic coordinates specified in Research Collaboratory for Structural Bioinformatics (RCSB) Protein Data Bank accession code 3O41;

wherein the antibody-binding domain of the immunogen comprises less than 12 consecutive amino acids from a motavizumab-binding domain or a 101F antibody-binding domain of RSV F protein, and wherein the immunogen elicits a humoral immune response against RSV. In one embodiment, the antibody-binding domain of the immunogen comprises contact residues that have a spatial orientation represented by atomic coordinates that have a root mean square deviation of protein backbone atoms of less than 10 angstroms from the corresponding backbone atoms of contact residues in an RSV F peptide that contacts motavizumab or 101F antibody in the respective complex. In one embodiment, the immunogen comprises contact residues of the motavizumab-binding domain embedded in a protein scaffold comprising a three-dimensional structure having two alpha helices defined by atomic coordinates that have a root mean square deviation of protein backbone atoms of less than 10 angstroms when superimposed on the two alpha helices of the peptide consisting of amino acid sequence SEQ ID NO:2 when complexed with motavizumab, the three-dimensional model of the complex being defined by the coordinates specified in Protein Data Bank accession code 3IXT. In one embodiment, the immunogen comprises contact residues of the 101F antibody-binding domain embedded in a protein scaffold comprising a three-dimensional structure with atomic coordinates that have a root mean square deviation of protein backbone atoms of less than 10 angstroms when superimposed on the peptide consisting of amino acid sequence SEQ ID NO:4 when complexed with 101F antibody, the 3-dimensional model of the complex being defined by the coordinates specified in Protein Data Bank accession code 3O41. The present disclosure also provides a nucleic acid molecule comprising a nucleic acid sequence that encodes any of such immunogens. Also provided is a recombinant molecule comprising such a nucleic acid molecule. Also provided is a recombinant cell comprising such a recombinant molecule. Also provided are uses of such nucleic acid molecules, recombinant molecules, and recombinant cells to produce any of such immunogens. A recombinant molecule can also be a nucleic acid vaccine. Also provided is a composition that can include any of the immunogens, nucleic acid molecules, recombinant molecules, or recombinant cells. Also provided is a vaccine comprising any of such immunogens. The disclosure provides a method to elicit a neutralizing humoral immune response against RSV; such method comprises administering any of such immunogens or vaccines, wherein such administration elicits a neutralizing humoral immune response against RSV. Also provided is a method to protect a patient from RSV infection; such method comprises administering to the patient any of such immunogens or vaccines, wherein such administration protects the patient from RSV infection.

The disclosure provides an antibody protein comprising a heavy chain comprising SEQ ID NO:5, except that the antibody protein comprises at least one amino acid substitution selected from the group consisting of: (a) substitution of the amino acid corresponding to position 32 of SEQ ID NO:5, is a histidine or a glutamic acid; (b) substitution of the amino acid at position 35 of SEQ ID NO:5 is substituted with an alanine; (c) substitution of the amino acid at position 52 of SEQ ID NO:5 is substituted with an amino acid selected from the group consisting of lysine, histidine, threonine, serine and arginine; (d) substitution of the amino acid at position 53 of SEQ ID NO:5 is substituted with a histidine or a serine; (e) substitution of the amino acid at position 54 of SEQ ID NO:5 is substituted with a phenylalanine or an arginine; (f) substitution of the amino acid at position 56 of SEQ ID NO:5 is substituted with an amino acid selected from the group consisting of isoleucine, serine, glutamic acid, and aspartic acid; (g) substitution of the amino acid at position 58 of SEQ ID NO:5 is substituted with a tyrosine or a tryptophan; (h) substitution of the amino acid at position 97 of SEQ ID NO:5 is substituted with an aspartic acid, a histidine or an arginine; (i) substitution of the amino acid at position 99 of SEQ ID NO:5 is substituted with aspartic acid; (j) substitution of the amino acid at position 100 of SEQ ID NO:5 is substituted with a tyrosine, a tryptophan or a histidine; and (k) substitution of the amino acid at position 100A of SEQ ID NO:5 is substituted with a serine or a threonine. The disclosure provides an antibody protein comprising a heavy chain comprising SEQ ID NO:6, except that the antibody protein comprises at least one amino acid substitution selected from the group consisting of: (a) substitution of the amino acid at position 32 of SEQ ID NO:6 is substituted with a phenylalanine; (b) substitution of the amino acid at position 49 of SEQ ID NO:6 is substituted with a histidine or an arginine; (c) substitution of the amino acid at position 92 of SEQ ID NO:6 is substituted with a lysine; (d) substitution of the amino acid at position 94 of SEQ ID NO:6 is substituted with a histidine; and (e) substitution of the amino acid at position 96 of SEQ ID NO:6 is substituted with a histidine. The present disclosure also provides a nucleic acid molecule comprising a nucleic acid sequence that encodes any of such antibody proteins. Also provided is a recombinant molecule comprising such a nucleic acid molecule. Also provided is a recombinant cell comprising such a recombinant molecule. Also provided are uses of such nucleic acid molecules, recombinant molecules, and recombinant cells to produce any of such antibody proteins. Also provided is a composition that can include any of the antibody proteins. Also provided is a method to protect a patient from RSV infection; such method comprises administering to the patient any of such immunogens or vaccines, wherein such administration protects the patient from RSV infection.

### BRIEF DESCRIPTION OF THE FIGURES

FIG. 1. Structural basis of motavizumab binding to its F glycoprotein epitope. (a) RSV neutralization curves for palivizumab and motavizumab IgG as determined by a flow cytometric assay. (b) Surface representation of the Fab and ribbon representation of the peptide, viewed looking down at the CDRs. (c) Interactions between the peptide and six Fab CDRs. Side chains are shown for those residues making intermolecular interactions. Dashed lines represent hydrogen bonds. Residues in the hydrophobic patch on the heavy chain are shown with transparent surfaces. These include Trp52 and Trp53 on the right, and Ile97, Phe98 and Phe100 on the left (d) Motavizumab mutations that alter affinity to the F glycoprotein. Side chains are shown for those Fab residues that increase affinity by directly contacting the peptide (red), by altering the position of residues that directly contact the peptide (cyan), and by binding to the F glycoprotein outside the primary epitope or enhancing long-range electrostatic interactions (magenta). Fab residues in yellow decrease the affinity for the F glycoprotein but increase in vivo potency compared to an earlier version of motavizumab.

FIG. 2. Motavizumab binding to the RSV F glycoprotein. (a) Superposition of the motavizumab-bound peptide (grey) and residues 229-252 of the PIV5 F glycoprotein structure (red). (b) Ribbon representation of the model of motavizumab Fab (green and blue) bound to the PIV5 F glycoprotein monomer (tan) via the superposition shown in a. (c) Same as b, except the entire PIV5 F glycoprotein trimer is shown (tan, green, pink). (d) Magnification of the boxed area shown in c. (e) Gel filtration elution profile and corresponding Coomassie blue-stained SDS-PAGE gel of the RSV F₀ Fd glycoprotein. (f) Gel filtration elution profile and corresponding Coomassie blue-stained SDS-PAGE gel of a mixture of RSV Fₒ Fd glycoprotein and excess palivizumab Fab. Densitometric analysis of the gel yields a ratio of 2.97 Fabs per trimer.

FIG. 3. Peptide electron density. Stereo image of 2Fₒ—F_{c} density contoured at 1σ around the 24-amino acid peptide, viewed from the bound antibody. The electrostatic potential of the peptide in this orientation is shown in FIG. 4 (Right).

FIG. 4. Shape and electrostatic potential complementarity. (Left) Cartoon and stick representation of the peptide bound to a surface of the motavizumab Fab colored according to electrostatic potentials (negative potentials are colored red, positive potentials are colored blue). (Right) Cartoon and stick representation of the six motavizumab CDRs bound to a surface of the peptide colored according to electrostatic potentials. Heavy chain CDRs are green, and light chain CDRs are blue. The scale is from −5 to 5 kT/e. The images are related by a 180° rotation.

FIG. 5. RSV/PIV5 F glycoprotein alignments. FIG. 5-1 (also labeled “a”) Sequence alignment. Sequence of the crystallized PIV5 F glycoprotein fragment aligned with the corresponding residues from the RSV F glycoprotein A2 strain. The sequences were first aligned using CLUSTALW2 (Larkin M A et al., 2007, Bioinformatics 23, 2947-2948) and then manually adjusted to align the furin cleavage site and disulfide bonds. Secondary structure of the PIV5 F glycoprotein as determined from the crystal structure and the PSIPRED-predicted (Bryson K et al., 2005, Nucl. Acids Res. 33, W36-38) RSV F and PIV5 F glycoprotein secondary structure is shown. The motavizumab epitope is highlighted in grey. Arrows indicate β-strands and coils indicate α-helices. Numbering corresponds to the RSV F glycoprotein. FIG. 5-2 (also labeled “b”) Structure alignment. The structure of the motavizumab-bound RSV F peptide was used to refine the alignment with the PIV5 F glycoprotein. Superposition of RSV F residues 254-277 with PIV5 F residues 229-252 provided an rmsd of 2.1 Å for all 24 Cα atoms. This optimal structure alignment requires a shift of 3 amino acids relative to the sequence alignment in a, which also results in a better sequence alignment for this region.

FIG. 6. RSV F0 Fd cross-linking Coomassie-stained SDS-PAGE gel of glutaraldehyde cross-linked RSV F₀ Fd proteins immunoprecipitated with motavizumab IgG.

FIG. 7. Comparison of the motavizumab epitope to Protein A and the 1lp1b scaffold. The 24-amino acid sequence of the motavizumab epitope on RSV F is shown on top, with the two helical regions identified in the crystal structure indicated by blue and orange cylinders. The sequence of a single domain of Protein A from S. aureus is shown (labeled Original), as well as the sequence of the 1lp1b_{—}003 scaffold (labeled Final). Bold residues were mutated to preserve motavizumab contact residues, while underlined residues were mutated to stabilize the conformation of the two helices. Structures of the motavizumab/peptide complex and the scaffold are shown below the sequences.

FIG. 8. Recombinant immunogen structure. Expression of immunogen 1lp1b_{—}003 in HEK293 and bacterial cells yields proteins that are alpha-helical in solution and have a melting temperature that confirms the proteins are folded in solution.

### DETAILED DESCRIPTION

Previous attempts at making an RSV vaccine using the RSV F protein have been unsuccessful. This was due to poor immunogenicity or a concern about eliciting T cells that could worsen disease. The inventors have now solved the crystal structures of two, antibody-binding domains of the F protein when they are bound to their respective monoclonal antibodies: motavizumab, which binds to the F protein at a domain spanning amino acids 254 to 277 (see, e.g., Wu H et al., 2007, J. Mol. Biol. 368, 652-665); and chimeric 101F antibody (also referred to as 101F or CH101 (Centocor)) that binds to the F protein at a domain spanning from amino acids 422-436 (see, e.g., Wu, S-J et al., 2007, J. Gen Virol 88, 2719-2723). Analysis of this structure has led to the identification of the contact residues in the F protein when bound to motavizumab or 101F antibody. This information allows the identification of non-RSV proteins that have a similar three-dimensional structure to the respective antibody-binding domains (referred to as scaffold proteins), which can then be modified to contain the appropriate residues that enable the modified protein to bind motavizumab or 101F. Since such a modified protein is unrelated to the RSV F protein, except for the contact residues, it can be used as an immunogen to elicit antibodies against the RSV F protein. Preferably the immunogens do not elicit a significant cellular response against the F protein. The information gained from the three-dimensional model can also be used to design antibodies that have a high affinity for the RSV F protein, and that can be used to protect individuals from RSV infection.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

It should be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It should be understood that as used herein, the term “a” entity or “an” entity refers to one or more of that entity. For example, a peptide or protein refers to one or more peptides or proteins. As such, the terms “a”, “an”, “one or more” and “at least one” can be used interchangeably. Similarly the terms “comprising”, “including” and “having” can be used interchangeably. Moreover, as used herein, the terms about and substantially refer to a variation of less than 5% from the object of the term, and preferably less than 2%.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed. In addition, all sub-combinations are also specifically embraced by the present invention and are disclosed herein just as if each and every such sub-combination was individually and explicitly disclosed herein.

One embodiment of the present invention is an immunogen comprising an antibody-binding domain, wherein the three-dimensional structure of the antibody-binding domain of the immunogen spatially corresponds to the three-dimensional structure of an antibody-binding domain of a peptide derived from RSV F protein, when such peptide is in a complex with an RSV neutralizing antibody that specifically binds the F protein. In one embodiment, the neutralizing antibody is palivizumab (SYNAGIS®). In another embodiment, the neutralizing antibody is motavizumab. In yet another embodiment, the neutralizing antibody is 101F. In one embodiment, the neutralizing antibody can be any antibody that binds an antibody-binding domain recognized by motavizumab (i.e., an antibody-binding domain to which motavizumab binds). In one embodiment, the neutralizing antibody can be any antibody that binds an antibody-binding domain recognized by 101F antibody (also referred to herein as 101F).

As used herein, a peptide derived from the RSV F protein is any peptide comprising at least a portion of SEQ ID NO:1, wherein said portion comprises an antibody-binding domain that binds palivizumab, motavizumab or 101F. Such a peptide can also be referred to as an RSV F peptide. As used herein, an antibody-binding domain is a group, or cluster, of amino acids within a protein or peptide, wherein at least one of the amino acid residues in the sequence interacts directly, or indirectly (e.g., forms a bond, such as an ionic bond or salt-bridge) with at least one amino acid residue in an antibody such as palivizumab, motavizumab or 101F, such that the antibody specifically binds the peptide. As used herein, the terms selectively, selective, specific, and the like, indicate the antibody has a greater affinity for the RSV protein or peptide, or the immunogen, than it does for proteins unrelated to the RSV F protein or peptide. More specifically, the terms selectively, selective, specific, and the like indicate that the affinity of the antibody the RSV protein or peptide, or the immunogen, is statistically significantly higher than its affinity for a negative control (e.g., an unrelated protein) as measured using a standard assay (e.g., ELISA). Suitable techniques for assaying the ability of an antibody to selectively interact with the RSV protein or peptide, or the immunogen, are known to those skilled in the art. Amino acid residues that act directly or indirectly to form bonds at the interface of two molecules, such as a peptide and antibody, are referred to as contact residues. Contact residues within a molecule can be contiguous, non-contiguous, or partly contiguous in the two-dimensional (linear) structure (i.e., linearly contiguous, linearly non-contiguous, or the like), but are sufficiently contiguous, or close together, in the three-dimensional structure to form an epitope (i.e., structurally contiguous).

In one embodiment, the peptide comprises a palivizumab-binding domain. In another embodiment, the peptide comprises a motavizumab antibody-binding domain (also referred to herein as a motavizumab antibody-binding site, motavizumab-binding domain, or motavizumab-binding site). In a preferred embodiment, the motavizumab antibody-binding site corresponds to amino acids 254-277 of SEQ ID NO:1. The amino acid sequence spanning residues 254-277 is NSELLSLIND MPITNDQKKL MSNN, also denoted herein as SEQ ID NO:2.

In yet another embodiment the peptide comprises a 101F antibody-binding site (also referred to herein as a 101F antibody-binding domain). In another preferred embodiment, the 101F antibody-binding site corresponds to amino acids 422-436 of SEQ ID NO:1. The amino acid sequence spanning residues 422-436 is STASNKNRGI IKTFS, also denoted herein as SEQ ID NO:3, except that the S at amino acid position 422 is replaced by a C in RSV F protein. In yet another preferred embodiment, the 101F antibody-binding site corresponds to amino acids 427-436 of SEQ ID NO:1. The amino acid sequence spanning residues 427-436 is KNRGIIKTFS, also denoted herein as SEQ ID NO:4. In yet another preferred embodiment, the 101F antibody-binding site corresponds to amino acids 422-438 of SEQ ID NO:1. The amino acid sequence spanning residues 422-438 is STASNKNRGI IKTFSNG, also denoted herein as SEQ ID NO:9, except that the S at amino acid position 422 is replaced by a C in RSV F protein.

A preferred embodiment of the present invention is an immunogen comprising an antibody-binding domain, wherein the three-dimensional structure of such antibody-binding domain spatially corresponds to the three-dimensional structure of a peptide consisting of amino acids 254-277 from the RSV F protein, when such peptide is complexed with motavizumab. Another preferred embodiment of the present invention is an immunogen comprising an antibody-binding domain, wherein the three-dimensional structure of such antibody-binding domain spatially corresponds to the three-dimensional structure of a peptide consisting of amino acids 427-436 from the RSV F protein, when such peptide is complexed with 101F. Another preferred embodiment of the present invention is an immunogen comprising an antibody-binding domain, wherein the three-dimensional structure of such antibody-binding domain spatially corresponds to the three-dimensional structure of a peptide consisting of amino acids 427-438 from the RSV F protein, when such peptide is complexed with 101F.

As used herein, the terms spatially corresponds, spatially corresponding, and the like, are used to indicate that when a three-dimensional model of a protein is superimposed on a three dimensional model of a RSV F peptide comprising a motavizumab or 101F binding domain when such peptide is in a complex with motavizumab or 101F, respectively, coordinates defining the spatial position of backbone atoms in the protein vary from coordinates defining the spatial location of analogous backbone atoms in the antibody-binding domain of the RSV F peptide, when such peptide is in a complex with motavizumab, by less than about 10 angstroms. Backbone atoms are those atoms in an amino acid that form the peptide backbone, or 3-dimensional folding pattern, of the 3-dimensional model. As such, backbone atoms are those atoms that make up the base, but not the side chain, of amino acid residues in s protein (i.e., nitrogen, carbon, alpha carbon, and oxygen). Analogous backbone atoms are atoms, that are in the same position within an amino acid. The term spatial position refers to an object's location in three-dimensional space, as defined by X, Y and Z coordinates. One system for determining the three-dimensional structure of a protein is X-ray crystallography. It is understood by those skilled in the relevant art that three-dimensional structures of proteins are defined using atomic coordinates. Thus, in one embodiment of the present invention the three-dimensional structure of the complex between the peptide consisting of amino acid sequence SEQ ID NO:2 and motavizumab is defined by the atomic coordinates recited, or specified, in Protein Data Bank (of the Research Collaboratory for Structural Bioinformatics (RCSB) accession code 3IXT (i.e., the atomic coordinates deposited at the Protein Data Bank under accession code 3IXT; also referred to as PDB acc code 3IXT). These coordinates were recited in Table 1 of U.S. Provisional Patent Application No. 61/253,826, filed Oct. 21, 2009 (U.S. 61/253,826). In another embodiment of the present invention, the three-dimensional structure of a complex between a peptide consisting of amino acid sequence SEQ ID NO:3 and 101F is defined by the atomic coordinates recited, or specified, in Protein Data Bank (of the Research Collaboratory for Structural Bioinformatics (RCSB) accession code 3O41 (also referred to as PDB acc code 3O41). These coordinates are more highly refined atomic coordinates corresponding to the atomic coordinates recited in Table 2; such refinement led to a three-dimensional structure that when superimposed on the three-dimensional structure of the complex defined by the atomic coordinates recited in Table 2 could not be distinguished visually from the latter structure; any differences were less than 0.1 angstroms. In another embodiment of the present invention, the three-dimensional structure of a complex between a peptide consisting of amino acid sequence SEQ ID NO:9 and 101F is defined by the atomic coordinates specified in Protein Data Bank (of the Research Collaboratory for Structural Bioinformatics (RCSB), accession code 3O45 (also referred to as PDB acc code 3O45).

While an immunogen of the present invention comprises an antibody binding domain spatially corresponding to an antibody-binding domain from an RSV F peptide present in a complex defined by the coordinates in PDB acc code 3IXT or in PDB acc code 3O41, it should be understood that some small variance in the spatial orientation of the immunogen antibody binding domain is permissible, as long as the immunogen binds palivizumab, motavizumab, or 101F. Thus one embodiment of the present invention is an immunogen comprising an antibody binding domain that has a three-dimensional structure defined by atomic coordinates having less than 10%, less than 5%, less than 3%, less than 2% or less than 1% variation from the atomic coordinates recited in PDB acc code 3IXT or in PDB acc code 3O41. Another embodiment of the presenting invention is an immunogen comprising an antibody-binding domain having a three-dimensional structure represented by atomic coordinates defining backbone atoms that have a root mean square deviation of less than 10 angstroms, less than 5 angstroms, less than 3 angstroms, less than 2 angstroms, or less than 1 angstrom from the backbone atoms of the antibody-binding domain of an RSV F peptide in a complex defined by the coordinates recited in PDB acc code 3IXT or in PDB acc code 3O41. Yet another embodiment of the present invention is an immunogen comprising an antibody-binding domain having a three-dimensional structure represented by atomic coordinates defining backbone atoms, wherein each atom has a root mean square deviation of less than 0.4 angstroms, less than 0.3 angstroms, less than 0.2 angstroms, or less than 0.1 angstrom from the corresponding backbone atom in the antibody-binding domain of an RSV F peptide in a complex defined by the coordinates recited in PDB acc code 3IXT or in PDB acc code 3O41.

As disclosed herein, an immunogen of the present invention comprises an antibody-binding domain that spatially corresponds to the antibody-binding domain in the RSV F peptide when bound to motavizumab or 101F. It is preferable that an immunogen of the present invention contain little or no homology to RSV F protein sequences from outside of an antibody-binding domain. It is also preferable that the immunogen not include any contiguous sequence from RSV F protein that is of sufficient length to generate a cellular immune response. As used herein, a cellular immune response, or cell-mediated immunity, refers to a T lymphocyte immune response and the release of related cytokines and other immunomodulatory molecules in response to an antigen that contains an antigenic peptide fragment consisting of a specific sequence of about 10 amino acids. In contrast, a humoral immune response, or humoral immunity, refers to the production by B-lymphocytes of antibodies (e.g., IgG, IgM or IgA antibodies) in response to an antigen. Such antibodies preferably neutralize RSV. One embodiment of the present invention is an immunogen that elicits a humoral immune response, but not a significant cellular immune response against RSV. One embodiment of the present invention is an immunogen that elicits a humoral immune response, but not a cellular immune response against RSV.

In one embodiment, an immunogen of the present invention comprises less than 12 consecutive (also referred to herein as contiguous or adjacent) amino acids from the sequence of the RSV F protein. In one embodiment, an immunogen of the present invention comprises less than 11 consecutive amino acids from the sequence of the RSV F protein. In one embodiment, an immunogen of the present invention comprises less than 10 consecutive amino acids from the sequence of the RSV F protein. In one embodiment, an immunogen of the present invention comprises less than 9 consecutive amino acids from the sequence of the RSV F protein. In one embodiment, an immunogen of the present invention comprises less than 9, less than 8, less than 7, less than 6, less than 5, less than 4, or less than 3 contiguous amino acids from the sequence of the RSV F protein. In one embodiment, an immunogen of the present invention does not comprise an amino acid sequence from the RSV F protein that lies outside of the antibody-binding domain and that could elicit a cellular immune response to the RSV F protein. In a preferred embodiment, an immunogen of the present invention comprises less than 7, less than 6, less than 5, less than 4, or less than 3 contiguous amino acids from sequences outside of amino acids 254-277 from SEQ ID NO:1. In another preferred embodiment, an immunogen of the present invention comprises less than 7, less than 6, less than 5, less than 4, or less than 3 contiguous amino acids from outside of amino acids 427-436 from SEQ ID NO:1.

As described above, antibody-binding domains contain contact residues. As used herein, a contact residue is any amino acid present in a molecule (e.g., a peptide or antibody) that interacts directly or indirectly (e.g., forms an ionic bond either directly, or indirectly through a salt bridge), with an amino acid in a second molecule (e.g., a peptide or antibody), thereby resulting in formation of a complex between the two molecules. Preferably, immunogens of the present invention have contact residues capable of binding to the contact residues in motavizumab, or 101F, that are responsible for the binding of the antibody to the RSV F protein peptide. This disclosure provides immunogens of the embodiments that have contact residues capable of binding to the contact residues in motavizumab that are responsible for the binding of the antibody to the RSV F protein peptide. The disclosure also provides immunogens of the embodiment that have contact residues capable of binding to the contact residues in 101F that are responsible for the binding of the antibody to the RSV F protein peptide.

One embodiment of the present invention is an immunogen comprising a motavizumab-binding domain, wherein the contact residues within such motavizumab-binding domain have a spatial orientation represented by atomic coordinates defining backbone atoms, wherein each atom has a root mean square deviation of less than 10 angstroms, less than 5 angstroms, less than 2 angstroms, less than 1 angstrom, less than 0.4 angstroms, less than 0.3 angstroms, less than 0.2 angstroms, less or than 0.1 angstrom from the corresponding backbone atom in the antibody-binding domain of an RSV F peptide in a complex defined by the coordinates defined by the coordinates specified in PDB acc code 3IXT. In one embodiment, an immunogen comprises a motavizumab-binding domain that comprises contact residues that have a spatial orientation represented by atomic coordinates that have a root mean square deviation of protein backbone atoms of less than 10 angstroms from the corresponding backbone atoms of contact residues in an RSV F peptide that contacts motavizumab in a complex defined by the coordinates specified in PDB acc code 3IXT.

The disclosure includes an immunogen of the embodiments in which the motavizumab-binding domain from such immunogen comprises less than 15 amino acids of the motavizumab-binding domain from the RSV F peptide and in which such amino acids are in clusters of no more than 8 consecutive amino acids per cluster. The disclosure also includes an immunogen of the embodiments in which the motavizumab-binding domain from such immunogen comprises less than 15 amino acids of the motavizumab-binding domain from the RSV F peptide, and in which such amino acids are in clusters of no more than 3 consecutive amino acids per cluster. Also included are immunogens in which such amino acids are in clusters of no more than 7, 6, 5, 4, 2 or 1 consecutive amino acids per cluster.

Another embodiment of the present invention is an immunogen comprising an 101F-binding domain, wherein the contact residues within such 101F-binding domain have a spatial orientation represented by atomic coordinates defining backbone atoms, wherein each atom has a root mean square deviation of less than 10 angstroms, less than 5 angstroms, less than 2 angstroms, less than 1 angstrom, less than 0.4 angstroms, less than 0.3 angstroms, less than 0.2 angstroms, or less than 0.1 angstrom from the corresponding backbone atom in the antibody-binding domain of an RSV F peptide in a complex defined by the coordinates recited in PDB acc code 3O41. In one embodiment, an immunogen comprises a 101F antibody-binding domain that comprises contact residues that have a spatial orientation represented by atomic coordinates that have a root mean square deviation of protein backbone atoms of less than 10 angstroms from the corresponding backbone atoms of contact residues in an RSV F peptide that contacts 101F antibody in a complex defined by the coordinates recited in PDB acc code 3O41.

The disclosure includes an immunogen of the embodiments in which the 101F-binding domain from such immunogen comprises no more than 10 amino acids of the 101F-binding domain from the RSV F peptide, and in which such amino acids are in clusters of no more than 8 consecutive amino acids per cluster. Also included are immunogens in which such amino acids are in clusters of no more than 7, 6, 5, 4, 3, 2 or 1 consecutive amino acids per cluster.

In order to produce immunogens described herein, the inventors have developed novel methods of identifying proteins that comprise regions, referred to as superpositions, that spatially correspond to an antibody-binding domain from an RSV F peptide present in a complex defined by the coordinates in PDB acc code 3IXT or in PDB acc code 3O41. Such proteins are referred to as scaffolds, protein scaffolds, scaffold proteins, scaffold protein sequences, and the like. Scaffold proteins are useful for creating immunogens of the present invention in that they hold contact residues in the immunogen in the proper spatial orientation to facilitate interaction between such residues and contact residues of motavizumab, or 101F. Moreover, the selection criteria select only those proteins that substantially or wholly lack immunodominant RSV epitopes that would elicit a cellular immune response. This method, which is referred to as superpositioning, comprises determining the three-dimensional structure of an epitope of interest and then computationally searching a database of known protein structures to identify those proteins that can be structurally superimposed onto the epitope of interest with minimal root mean square deviation of their coordinates. Such a method can be accomplished using software such as, for example, ROSETTA. Superpositioning has been described in PCT International Publication No. WO 2008/025015 A2, published Feb. 28, 2008, which is hereby incorporated by reference in its entirety. Once suitable scaffold proteins have been identified, they can be altered according to the methods disclosed herein. A related method that can be used for the analysis of complex epitopes is referred to as double superpositioning. In this method, which is similar to superpositioning, scaffolds are scanned for structural similarity to each of the two epitope segments individually, and whenever a match is found to one of the segments, that scaffold is searched a second time for structural similarity to the other epitope segment, with the rigid body position of the second epitope segment relative to the scaffold pre-determined by the superposition of the first segment to the scaffold. “Double” matches are identified if (a) one epitope segment matches a scaffold with backbone rmsd/nsup<threshold 1 and the other segment matches with backbone rmsd/nsup<threshold 2, where threshold 1 is typically 0.15 and threshold 2 is typically 0.2, or (b) if both segments are superimposed simultaneously onto the scaffold and the backbone rmsd/nsup is <threshold 3, where threshold 3 is typically 0.2. In this instance, the term “backbone rmsd” is defined as the root-mean square deviation of a structural alignment computed over the backbone atoms N, CA, C, O; and “backbone rmsd/nsup” is defined as the “backbone rmsd” divided by the number of aligned residues.

One embodiment of the present invention is an immunogen in which contact residues of the motavizumab, or 101F, binding domain, are embedded in a protein scaffold that spatially corresponds to an antibody-binding domain from an RSV F peptide present in a complex defined by the coordinates in PDB acc code 3IXT or in PDB acc code 3O41, respectively. Such a protein scaffold can be identified using the three-dimensional structure of a complex described by the coordinates in PDB acc code 3IXT or in PDB acc code 3O41. As used herein, embedding of contact residues in a protein scaffold refers to positioning contact residues within the scaffold such that such contact residues form an antibody-binding domain and such that the protein scaffold retains its proper three-dimensional structure.

Analysis of the three-dimensional structure of the complex of the F peptide bound to motavizumab described by the coordinates in PDB acc cod 3IXT shows that the contact residues are embedded in a three-dimensional structure comprising two alpha helices. Thus, one embodiment of the present invention is an immunogen comprising an antibody-binding domain that binds motavizumab, wherein the contact residues in such antibody-binding domain are embedded in a protein scaffold comprising a three-dimensional structure that has two alpha helices, wherein the helices are defined by atomic coordinates defining backbone atoms, wherein each atom has a root mean square deviation of less than 10 angstroms, less than 5 angstroms, less than 2 angstroms, less than 1 angstrom, less than 0.4 angstroms, less than 0.3 angstroms, less than 0.2 angstroms, or less than 0.1 angstrom from the corresponding backbone atom in the antibody-binding domain of an RSV F peptide in a complex defined by the coordinates recited in PDB acc cod 3IXT. In one embodiment, the alpha helices consist of amino acids 2-10 and amino acids 15-23 of the F peptide.

The disclosure provides an immunogen of the embodiments in which contact residues of the motavizumab-binding domain are embedded in a protein scaffold comprising a three-dimensional structure having two alpha helices defined by atomic coordinates that have a root mean square deviation of protein backbone atoms of less than 10 angstroms when superimposed on the two alpha helices of the peptide consisting of amino acid sequence SEQ ID NO:2 when complexed with motavizumab, the three-dimensional model of such complex being defined by the coordinates specified in PDB acc cod 3IXT. The disclosure also provides an immunogen of the embodiments in which contact residues of the 101F antibody-binding domain are embedded in a protein scaffold comprising a three-dimensional structure with atomic coordinates that have a root mean square deviation of protein backbone atoms of less than 10 angstroms when superimposed on the peptide consisting of amino acid sequence SEQ ID NO:4 when complexed with 101F antibody, the 3-dimensional model of such complex being defined by the coordinates specified in PDB acc code 3O41. The disclosure also provides an immunogen of the embodiments in which contact residues of the 101F antibody-binding domain are embedded in a protein scaffold comprising a three-dimensional structure with atomic coordinates that have a root mean square deviation of protein backbone atoms of less than 10 angstroms when superimposed on the peptide consisting of amino acid sequence SEQ ID NO:9 when complexed with 101F antibody, the 3-dimensional model of such complex being defined by the coordinates specified in PDB acc code 3O45.

As has been described, scaffold proteins are identified by their spatial similarity to the three-dimensional structure of a motavizumab or 101F binding domain. Moreover, preferable scaffold sequences do not share significant homology with the RSV F protein (i.e., they do not elicit a cellular immune response against RSV). Thus, using the techniques described herein, the inventors have now identified several proteins that can serve as scaffolds for creating motavizumab-binding and 101F-binding immunogens. Examples of such proteins include, but are not limited to, Staphylococcus aureus protein, Helicobacter pylori CagZ protein, and equine infectious anemia virus p26 protein. One embodiment is a scaffold protein having PDB (Protein Data Bank) accession code 1LP1, preferably scaffold 1LP1_b (SEQ ID NO:11). Another embodiment is a scaffold protein having PDB accession code 1S2X, preferably scaffold 1S2X_a (SEQ ID NO:12). One embodiment is a truncated 1 S2X_a scaffold that has SEQ ID NO:13; this scaffold is truncated at the carboxyl terminus. Another embodiment is a scaffold protein having PDB accession code 2EIA, preferably scaffold 2EIA_a (SEQ ID NO:14).

It will be understood by those in the relevant art that while a protein scaffold may comprise a three-dimensional structure capable of holding contact residues in the correct spatial position, and since such a scaffold protein may be unrelated to the RSV F protein, the scaffold protein itself may not contain amino acids that spatially correspond to contact residues in the F protein. Consequently, an unmodified, scaffold protein may not be able to bind to motavizumab or 101F. As used herein the term unmodified scaffold protein is a scaffold protein represented by a three-dimensional model, a portion of which spatially corresponds to the antibody-binding domain of an RSV F protein in a complex defined by the atomic coordinates in PDB acc cod 3IXT or in PDB acc cod 3O41, but which has not been altered to contain any of the contact residues present in the RSV F protein. Amino acids in the RSV F protein identified as interacting with the contact residues in motavizumab are the amino acids at positions 255, 258, 259, 261, 262, 263, 267, 268, 269, 271, 272, 273, 275 and 276 of SEQ ID NO:1. Amino acids in the RSV F protein identified as interacting with the contact residues in 101F are the amino acids at positions 427, 429, 431, 432, 433, 434, 435 and 436 of SEQ ID NO:1. Thus one embodiment of the present invention is an immunogen that comprises sequence from a scaffold protein, wherein at least one amino acid in such scaffold protein sequence spatially corresponding to a contact residue in the RSV F protein is substituted with the amino acid residue present at such spatially corresponding contact residue in the F peptide. Such an immunogen can be produced by recombinant methods and/or synthesizing a nucleic acid molecule that encodes such immunogen and expressing it to make a recombinant immunogen. Such an immunogen can be tested for efficacy by measuring the immunogen's ability to bind to its respective antibody, or to neutralize RSV, using techniques known to those skilled in the art.

One preferred embodiment of the present invention is a scaffold protein comprising at least one substitution selected from the group consisting of:

(a) substituting a serine for the amino acid spatially corresponding to the amino acid at position 2 of SEQ ID NO:2;

(b) substituting a leucine for the amino acid spatially corresponding to the amino acid at position 5 of SEQ ID NO:2;

(c) substituting a serine for the amino acid spatially corresponding to the amino acid at position 6 of SEQ ID NO:2;

(d) substituting a isoleucine for the amino acid spatially corresponding to the amino acid at position 8 of SEQ ID NO:2;

(e) substituting a asparagine for the amino acid spatially corresponding to the amino acid at position 9 of SEQ ID NO:2;

(f) substituting a aspartic acid for the amino acid spatially corresponding to the amino acid at position 10 of SEQ ID NO:2;

(g) substituting a threonine for the amino acid spatially corresponding to the amino acid at position 14 of SEQ ID NO:2;

(h) substituting a asparagine for the amino acid spatially corresponding to the amino acid at position 15 of SEQ ID NO:2;

(i) substituting a aspartic acid for the amino acid spatially corresponding to the amino acid at position 16 of SEQ ID NO:2;

(j) substituting a lysine for the amino acid spatially corresponding to the amino acid at position 18 of SEQ ID NO:2;

(k) substituting a lysine for the amino acid spatially corresponding to the amino acid at position 19 of SEQ ID NO:2;

(l) substituting a leucine for the amino acid spatially corresponding to the amino acid at position 20 of SEQ ID NO:2;

(m) substituting a serine for the amino acid spatially corresponding to the amino acid at position 22 of SEQ ID NO:2; and

(n) substituting a asparagine for the amino acid spatially corresponding to the amino acid at position 23 of SEQ ID NO:2.

One embodiment is a scaffold protein comprising all of the afore-mentioned substitutions.

Another preferred embodiment of the present invention is a scaffold protein comprising at least one substitution selected from the group consisting of:

(a) substituting a lysine for the amino acid spatially corresponding to the amino acid at position 1 of SEQ ID NO:4;

(b) substituting a arginine for the amino acid spatially corresponding to the amino acid at position 3 of SEQ ID NO:4;

(c) substituting a isoleucine for the amino acid spatially corresponding to the amino acid at position 5 of SEQ ID NO:4;

(d) substituting a isoleucine for the amino acid spatially corresponding to the amino acid at position 6 of SEQ ID NO:4;

(e) substituting a lysine for the amino acid spatially corresponding to the amino acid at position 7 of SEQ ID NO:4;

(f) substituting a threonine for the amino acid spatially corresponding to the amino acid at position 8 of SEQ ID NO:4;

(g) substituting a phenylalanine for the amino acid spatially corresponding to the amino acid at position 9 of SEQ ID NO:4; and,

(h) substituting a serine for the amino acid spatially corresponding to the amino acid at position 10 of SEQ ID NO:4.

One embodiment is a scaffold protein comprising all of the afore-mentioned substitutions.

In one embodiment, an immunogen comprises amino acids 2, 5, 6, 8, 9, 16, 18, 19, 20, 22 and 23 of SEQ ID NO:2 substituted at the spatially corresponding positions of a scaffold protein. In one embodiment, an immunogen comprises amino acids 2, 5, 6, 9, 16, 18, 19, 20, 22 and 23 of SEQ ID NO:2 substituted at the spatially corresponding positions of a scaffold protein.

One embodiment of the present invention is an immunogen comprising an amino acid sequence of a protein selected from the group consisting of 1lp1b_{—}001 (SEQ ID NO:18), 1lp1b_{—}002 (SEQ ID NO:21), 1lp1b_{—}003 (SEQ ID NO:24), 1lp1b_{—}004 (SEQ ID NO:149), 1s2xa_{—}001 (SEQ ID NO:152), 1s2xa_{—}002 (SEQ ID NO:155), 1s2xa_{—}003 (SEQ ID NO:158), 1s2xa_{—}004 (SEQ ID NO:164), 2eiaa_{—}001 (SEQ ID NO:167), and 2eiaa_{—}002 (SEQ ID NO:170), the amino acid sequences of which are disclosed in the Examples. One embodiment is an immunogen comprising 1lp1b_{—}001 (SEQ ID NO:18). One embodiment is an immunogen comprising 1lp1b_{—}002 (SEQ ID NO:21). One embodiment is an immunogen comprising 1lp1b_{—}003 (SEQ ID NO:24). One embodiment is an immunogen comprising 1lp1b_{—}004 (SEQ ID NO:149). One embodiment is an immunogen comprising 1s2xa_{—}001 (SEQ ID NO:152). One embodiment is an immunogen comprising 1s2xa_{—}002 (SEQ ID NO:155). One embodiment is an immunogen comprising 1s2xa_{—}003 (SEQ ID NO:158). One embodiment is an immunogen comprising 1s2xa_{—}004 (SEQ ID NO:164). One embodiment is an immunogen comprising 2eiaa_{—}001 (SEQ ID NO:167). One embodiment is an immunogen comprising 2eiaa_{—}002 (SEQ ID NO:170). One embodiment is a variant of any of such immunogens, wherein the variant shares at least 95%, at least 96%, at least 97%, at least 98%, or at least 99% amino acid sequence identity with such immunogen. Preferably the variant retains contact residues of such immunogen.

One embodiment is an immunogen comprising an amino acid sequence of protein 1s2xa_{—}003_PADRE (SEQ ID NO:161). One embodiment is a variant of such immunogen, wherein the variant shares at least 95%, at least 96%, at least 97%, at least 98%, or at least 99% amino acid sequence identity with such immunogen. Preferably the variant retains contact residues of such immunogen.

One embodiment is an immunogen comprising an amino acid sequence of a protein selected from the group consisting of 1s2xa_{—}001_N_His (SEQ ID NO:177), 1s2xa_{—}002_N_His (SEQ ID NO:178), 1s2xa_{—}003_N_His (SEQ ID NO:179), 1s2xa_{—}004_N_His (SEQ ID NO:180), and 2eiaa_{—}002_N_His (SEQ ID NO:181), the amino acid sequences of which are disclosed in the Examples. One embodiment is a variant of any of such immunogens, wherein the variant shares at least 95%, at least 96%, at least 97%, at least 98%, or at least 99% amino acid sequence identity with such immunogen. Preferably the variant retains contact residues of such immunogen.

In addition to substituting amino acids in the portion of the scaffold protein spatially corresponding to the antibody-binding domain of RSV F peptide, other changes can be made to the immunogen as well, as long as such changes do not reduce the affinity of motavizumab or 101F for the immunogen. For example, amino acid residues outside of the antibody-binding domain may be altered in order to reduce steric interference between the backbone atoms of the antibody and the backbone atoms of the scaffold protein portion of the immunogen. In addition, amino acids that are outside of the antibody-binding domain can be substituted with an amino acid that allows the formation of a new ionic bond, thereby strengthening the interaction between the immunogen and the antibody. Such alteration of the scaffold protein is herein referred to as epitope conformation stabilization.

One embodiment of the invention is an immunogen comprising a motavizumab-binding domain spatially corresponding to a motavizumab-binding domain from an RSV F peptide, wherein the scaffold protein sequence of such immunogen has been subject to epitope stabilization conformation to reduce steric hindrance and/or increase the affinity of the immunogen for motavizumab. Another embodiment of the invention is an immunogen comprising a 101F-binding domain spatially corresponding to a 101F-binding domain from an RSV F peptide, wherein the scaffold protein sequence of such immunogen has been subject to epitope stabilization conformation to reduce steric hindrance and/or increase the affinity of the immunogen for 101F.

In addition to alterations of the immunogen sequence that result in reduced steric hindrance and/or increase the affinity of the immunogen for motavizumab of 101F, the immunogen can be subject to a process referred to as resurfacing, addition of N-linked glycosylation sites, or PEGylation. As used herein, the term resurfacing refers to a process whereby amino acid substitutions are introduced into scaffold sequences that are outside of the antibody-binding domain in order to eliminate or hide immunodominant epitopes. For example, amino acids within an immunodominant epitope can be substituted with neutral amino acids (i.e., having an uncharged R group) so that the epitope is no longer bound by an antibody. In some embodiments, N-linked glycosylation sites can be introduced into the protein, resulting in glycosylation of the immunogen such that immunodominant epitopes are hidden from the immune system and thus do not elicit a strong humoral or cell mediated immune response. In some embodiments, a scaffold can be PEGylated (i.e., treated with polyethylene glycol), or otherwise treated, to mask immunodominant epitopes. Such processes can also be referred to as cloaking. Methods of producing resurfaced proteins have been previously described in, for example, PCT International Publication No. WO/2009/100376 entitled, “Antigenic Cloaking and Its Use”, published Aug. 13, 2009, which is hereby incorporated by reference in its entirety. As used herein, the phrase immunodominant epitope refers to an epitope within a protein or peptide that is most easily recognized by the immune system and thus has the greatest influence on the specificity of an antibody elicited by a protein or peptide containing the immunodominant epitope.

One embodiment of the present invention is an immunogen comprising sequences from a scaffold protein, wherein such immunogen binds motavizumab or 101F, and wherein scaffold protein sequences outside of the antibody-binding domain of such immunogen have been subject to resurfacing. In one embodiment, amino acids in the scaffold protein sequences of the immunogen are substituted with neutral amino acids. In another embodiment, glycosylation sites are introduced into scaffold protein sequences of the immunogen, or the immunogen is submitted to PEGylation methodology such that immunodominant epitopes present in the immunogen are hidden from the immune system by glycosylation or PEGylation of the immunogen. It should be appreciated that immunogens of the present invention can comprise combinations of the amino acid alterations discussed above. Whether scaffold protein sequences will require the introduction of neutral amino acids, glycosylation or PEGylation or combinations of such types of alterations depends on the nature of the sequences present in the scaffold protein. It is within the ability of those skilled in the art to determine which alterations will best eliminate or hide immunodominant epitopes outside of the antibody-binding domain. Moreover, methods of substituting amino acids into a protein or peptide, introducing glycosylation sites into a protein or peptide or PEGylating such protein or peptide are known to those skilled in the art.

A preferred embodiment of the present invention is an immunogen comprising an amino acid sequence of a protein selected from the group consisting of mota_{—}1lp1b.m1.c1.d1_glyc1 (SEQ ID NO:55), mota_{—}1lp1b.m1.c1.d1_glyc2 (SEQ ID NO:56), mota_{—}1lp1b.m1.c1.d1_des1_{—}1 (SEQ ID NO:66), mota_{—}1lp1b.m1.c1.d1_des1_{—}2 (SEQ ID NO:67), mota_{—}1lp1b.m1.c1.d1_des1_{—}3 (SEQ ID NO:68), mota_{—}1lp1b.m1.c1.d1_des1_{—}5 (SEQ ID NO:69), mota_{—}1lp1b.m1.c1.d1_des1_{—}6 (SEQ ID NO:70), mota_{—}1lp1b.m1.c1.d1_des1_{—}7 (SEQ ID NO:71), mota_{—}1lp1b.m1.c1.d1_des1_{—}8 (SEQ ID NO:72), mota_{—}1lp1b.m1.c1.d1_des1_{—}9 (SEQ ID NO:73), mota_{—}1lp1b.m1.c1.d1_des1_{—}10 (SEQ ID NO:74), and mota_{—}1lp1b.m1.c1.d1_des1_{—}11 (SEQ ID NO:75), the amino acid sequences of which are disclosed in the Examples.

One embodiment is an immunogen that has one or more N-linked glycosylation sites, such as, but not limited to, mota_{—}1lp1b.m1.c1.d1 glyc1 (SEQ ID NO:55), mota_{—}1lp1b.m1.c1.d1_glyc2 (SEQ ID NO:56), 1lp1b_{—}003_Glyc1 (SEQ ID NO:39), 1lp1b_{—}003_Glyc2 (SEQ ID NO:42), 1lp1b_{—}003_Glyc3 (SEQ ID NO:45), 1lp1b_{—}003_Glyc4 (SEQ ID NO:48), 1lp1b_{—}003_Glyc5 (SEQ ID NO:51), or 1lp1b_{—}003_Glyc6 (SEQ ID NO:54). One embodiment is an immunogen comprising mota_{—}1lp1b.m1.c1.d1_glyc1 (SEQ ID NO:55). One embodiment is an immunogen comprising mota_{—}1lp1b.m1.c1.d1_glyc2 (SEQ ID NO:56). One embodiment is an immunogen comprising 1lp1b_{—}003_Glyc1 (SEQ ID NO:39). One embodiment is an immunogen comprising 1lp1b_{—}003_Glyc2 (SEQ ID NO:42). One embodiment is an immunogen comprising 1lp1b_{—}003_Glyc3 (SEQ ID NO:45). One embodiment is an immunogen comprising 1lp1b_{—}003_Glyc4 (SEQ ID NO:48). One embodiment is an immunogen comprising 1lp1b_{—}003_Glyc5 (SEQ ID NO:51). One embodiment is an immunogen comprising 1lp1b_{—}003_Glyc6 (SEQ ID NO:54). One embodiment is a variant of any of such immunogens, wherein the variant shares at least 95%, at least 96%, at least 97%, at least 98%, or at least 99% amino acid sequence identity with such immunogen. Preferably the variant retains contact residues of such immunogen.

One embodiment is an immunogen that has been resurfaced, such as, but not limited to, mota_{—}1lp1b.m1.c1.d1_des1_{—}1 (SEQ ID NO:66), mota_{—}1lp1b.m1.c1.d1_des1_{—}2 (SEQ ID NO:67), mota_{—}1lp1b.m1.c1.d1_des1_{—}3 (SEQ ID NO:68), mota_{—}1lp1b.m1.c1.d1_des1_{—}5 (SEQ ID NO:69), mota_{—}1lp1b.m1.c1.d1_des1_{—}6 (SEQ ID NO:70), mota_{—}1lp1b.m1.c1.d1_des1_{—}7 (SEQ ID NO:71), mota_{—}1lp1b.m1.c1.d1_des1_{—}8 (SEQ ID NO:72), mota_{—}1lp1b.m1.c1.d1_des1_{—}9 (SEQ ID NO:73), mota_{—}1lp1b.m1.c1.d1_des1_{—}10 (SEQ ID NO:74), or mota_{—}1lp1b.m1.c1.d1_des1_{—}11 (SEQ ID NO:75). Additional examples are 1lp1b_{—}003_Surf1 (SEQ ID NO:59), 1lp1b_{—}003_Surf6 (SEQ ID NO:62), and 1lp1b_{—}003_Surf8 (SEQ ID NO:65). One embodiment is an immunogen comprising mota_{—}1lp1b.m1.c1.d1_des1_{—}1 (SEQ ID NO:66). One embodiment is an immunogen comprising mota_{—}1lp1b.m1.c1.d1_des1_{—}2 (SEQ ID NO:67). One embodiment is an immunogen comprising mota_{—}1lp1b.m1.c1.d1_des1_{—}3 (SEQ ID NO:68). One embodiment is an immunogen comprising mota_{—}1lp1b.m1.c1.d1_des1_{—}5 (SEQ ID NO:69). One embodiment is an immunogen comprising mota_{—}1lp1b.m1.c1.d1_des1_{—}6 (SEQ ID NO:70). One embodiment is an immunogen comprising mota_{—}1lp1b.m1.c1.d1_des1_{—}7 (SEQ ID NO:71). One embodiment is an immunogen comprising mota_{—}1lp1b.m1.c1.d1_des1_{—}8 (SEQ ID NO:72). One embodiment is an immunogen comprising mota_{—}1lp1b.m1.c1.d1_des1_{—}9 (SEQ ID NO:73). One embodiment is an immunogen comprising mota_{—}1lp1b.m1.c1.d1_des1_{—}10 (SEQ ID NO:74). One embodiment is an immunogen comprising mota_{—}1lp1b.m1.c1.d1_des1_{—}11 (SEQ ID NO:75). One embodiment is an immunogen comprising 1lp1b_{—}003_Surf1 or 1lp1b_{—}003_Surf8. One embodiment is an immunogen comprising 1lp1b_{—}003_Surf1 (SEQ ID NO:59). One embodiment is an immunogen comprising 1lp1b_{—}003_Surf6 (SEQ ID NO:62). One embodiment is an immunogen comprising 1lp1b_{—}003_Surf8 (SEQ ID NO:65). One embodiment is a variant of any of such immunogens, wherein the variant shares at least 95%, at least 96%, at least 97%, at least 98%, or at least 99% amino acid sequence identity with such immunogen. Preferably the variant retains contact residues of such immunogen.

The disclosure provides for immunogens of the embodiments that are multivalent. Without being bound by theory, it is believed that multivalent immunogens can elicit enhanced neutralizing antibody responses. A multivalent immunogen of the embodiments is an immunogen of the disclosure that includes a particle enabling attachment of one or more immunogens. Such a particle can be of a material known to those skilled in the art. Examples of particles include, but are not limited to, ferritin, viral capsid proteins, virus-like particles, and other proteins that assemble into high-copy, large particles. Such attachment is accomplished so as to not significantly reduce the ability of an immunogen of the embodiments to elicit a neutralizing humoral response against RSV. Such attachment can be accomplished by covalently binding an immunogen to such a particle or can be accomplished by designing a nucleic acid molecule than encodes an immunogen of the embodiments and a particle, or subunit thereof. In one embodiment, a multivalent immunogen can be administered as a prime and/or boost. In one embodiment, a multivalent immunogen can be administered as a prime. In one embodiment, a multivalent immunogen can be administered as a boost.

One embodiment is an immunogen of the embodiments that is attached to ferritin. Ferritin, a globular protein complex consisting of 24 protein subunits, is a ubiquitous intracellular protein that stores iron and releases it in a controlled manner. The use of ferritin fusion proteins as vaccines has been described, for example, by Carter D C, et al., U.S. Pat. No. 7,097,841 B2, issued Aug. 29, 2006. One embodiment is a multivalent immunogen comprising an amino acid sequence of a protein selected from the group consisting of 1lp1b_{—}003 ferritin (SEQ ID NO:138), 1lp1b_{—}003_eumS (SEQ ID NO:140), 1lp1b_{—}003_eumSP (SEQ ID NO:142), 1lp1b_{—}003_eumL (SEQ ID NO:144), and 1lp1b_{—}003_eumLP (SEQ ID NO:146). One embodiment is an immunogen comprising 1lp1b_{—}003 ferritin (SEQ ID NO:138). One embodiment is an immunogen comprising 1lp1b_{—}003_eumS (SEQ ID NO:140). One embodiment is an immunogen comprising 1lp1b_{—}003_eumSP (SEQ ID NO:142). One embodiment is an immunogen comprising 1lp1b_{—}003_eumL (SEQ ID NO:144). One embodiment is an immunogen comprising 1lp1b3_eumLP (SEQ ID NO:146). One embodiment is a variant of any of such immunogens, wherein the variant shares at least 95%, at least 96%, at least 97%, at least 98%, or at least 99% amino acid sequence identity with such immunogen. Preferably the variant retains contact residues of such immunogen.

The disclosure also provides an immunogen comprising an amino acid sequence of a protein selected from the group consisting of 1lp1b_{—}003_K46A (SEQ ID NO:78), 1lp1b_{—}003_Q52A (SEQ ID NO:81), 1lp1b_{—}003_I13L_F27A (SEQ ID NO:87), 1lp1b_{—}003_L41I_L42V (SEQ ID NO:90), 1lp1b-003_L41I_L42A (SEQ ID NO:93), 1lp1b_{—}003_I13A (SEQ ID NO:105), 1lp1b_{—}003_L16A (SEQ ID NO:108), 1lp1b_{—}003_F27A (SEQ ID NO:111), 1lp1b_{—}003_L41A (SEQ ID NO:114), 1lp1b_{—}003_L42A (SEQ ID NO:117), and 1lp1b_{—}003_Neg1 (SEQ ID NO:132). One embodiment is an immunogen comprising an amino acid sequence of a protein selected from the group consisting of 1lp1b_{—}003_C43 (SEQ ID NO:33), 1lp1b_{—}003_C47 (SEQ ID NO:36), 1lp1b_{—}003_Glyc3 (SEQ ID NO:45), 1lp1b_{—}003_Glyc4 (SEQ ID NO:48), 1lp1b_{—}003_Glyc5 (SEQ ID NO:51), 1lp1b_{—}003_Glyc6 (SEQ ID NO:54), 1lp1b_{—}003_K46A_Q52A (SEQ ID NO:84), lp1b_{—}003_Glycine1 (SEQ ID NO:120), 1lp1b_{—}003_Glycine2 (SEQ ID NO:123), 1lp1b_{—}003_Pos1 (SEQ ID NO:126), 1lp1b_{—}003 Pos2 (SEQ ID NO:129), and 1lp1b_{—}003_Neg2 (SEQ ID NO:135). One embodiment is an immunogen comprising an amino acid sequence of a protein selected from the group consisting of 1lp1b_{—}003_I13A_L42A (SEQ ID NO:96), 1lp1b_{—}003 L19A (SEQ ID NO:99), and 1lp1b_{—}003_L19A_L41I (SEQ ID NO:102). Such immunogens differ from immunogen 1lp1b_{—}003 with respect to surface charge, glycosylation pattern and intrinsic flexibility. One embodiment is a variant of any of such immunogens, wherein the variant shares at least 95%, at least 96%, at least 97%, at least 98%, or at least 99% amino acid sequence identity with such immunogen. Preferably the variant retains contact residues of such immunogen.

One embodiment is an immunogen comprising an amino acid sequence of protein 1lp1b_{—}003_K272E (SEQ ID NO:30). This immunogen lacks a key contact residue of the motavizumab-binding domain.

One embodiment is an immunogen comprising an amino acid sequence of protein 1lp1b_{—}003 F2Y H15N (SEQ ID NO:27). This immunogen was expressed in HEK293 cells and complexed with motavizumab, crystallized, and a three-dimensional model defined therefrom.

The disclosure provides an RSV immunogen that comprises an amino acid sequence of 1LP1_b (SEQ ID NO: 11) having from one to twenty amino acid substitutions, wherein at least one amino acid substitution is selected from the group consisting of:

(a) substitution of a serine at amino acid position N-25 in SEQ ID NO:11;

(b) substitution of a leucine at amino acid position 1-28 in SEQ ID NO:11;

(c) substitution of a serine at amino acid position Q-29 in SEQ ID NO: 11;

(d) substitution of an isoleucine at amino acid position L-31 in SEQ ID NO:11;

(e) substitution of an asparagine at amino acid position K-32 in SEQ ID NO:11;

(f) substitution of an aspartic acid at amino acid position K-4 in SEQ ID NO:11;

(g) substitution of a lysine at amino acid position Q-6 in SEQ ID NO:11;

(h) substitution of a lysine at amino acid position Q-7 in SEQ ID NO:11;

(i) substitution of a leucine at amino acid position N-8 in SEQ ID NO:11;

(j) substitution of a serine at amino acid position F-10 in SEQ ID NO:11; and

(k) substitution of an asparagine at amino acid position Y-11 in SEQ ID NO:11. Such an RSV immunogen can comprise all of the substitutions (a) through (k). An RSV immunogen can elicit a humoral immune response against RSV. The phrase “from one to twenty amino acid substitutions” allows for substitutions beyond those that are specified in (a) through (k). In one embodiment, the three-dimension structure of the antibody-binding domain is maintained or improved when such substitution(s) are made. One embodiment is an RSV immunogen that comprises an amino acid sequence that has up to nine substitutions (i.e., any number ranging from 0 through 9 substitutions) in an amino acid sequence of a protein selected from the group consisting of 1lp1b_{—}001 (SEQ ID NO:18), 1lp1b_{—}002 (SEQ ID NO:21), 1lp1b_{—}003 (SEQ ID NO:24), and 1lp1b_{—}004 (SEQ ID NO:149). One embodiment is an RSV immunogen that comprises an amino acid sequence that has up to twelve substitutions in the amino acid sequence of protein 1lp1b_{—}003 (SEQ ID NO:24). One embodiment is an RSV immunogen that comprises an amino acid sequence that is at least 95% identical to an amino acid sequence of a protein selected from the group consisting of 1lp1b_{—}001 (SEQ ID NO:18), 1lp1b_{—}002 (SEQ ID NO:21), 1lp1b_{—}003 (SEQ ID NO:24), and 1lp1b_{—}004 (SEQ ID NO:149). One embodiment is an RSV immunogen that comprises an amino acid sequence that is at least 95% identical to the amino acid sequence of 1lp1b_{—}003 (SEQ ID NO:24). One embodiment is an RSV immunogen that comprises an amino acid sequence of a protein selected from the group consisting of 1lp1b_{—}001 (SEQ ID NO:18), 1lp1b_{—}002 (SEQ ID NO:21), 1lp1b_{—}003 (SEQ ID NO:24), and 1lp1b_{—}004 (SEQ ID NO:149). One embodiment is an RSV immunogen comprising 1lp1b_{—}003; such an immunogen comprises amino acid sequence SEQ ID NO:24. One embodiment is an RSV immunogen that is N-linked glycosylated. One embodiment is an RSV immunogen that comprises an amino acid sequence of a protein selected from the group consisting of mota_{—}1lp1b.m1.c1.d1_glyc1 (SEQ ID NO:55), mota_{—}1lp1b.m1.c1.d1_glyc2 (SEQ ID NO:56), 1lp1b_{—}003_Glyc1 (SEQ ID NO:39), 1lp1b_{—}003_Glyc2 (SEQ ID NO:42), 1lp1b_{—}003_Glyc3 (SEQ ID NO:45), 1lp1b_{—}003_Glyc4 (SEQ ID NO:48), 1lp1b_{—}003_Glyc5 (SEQ ID NO:51), and 1lp1b_{—}003_Glyc6 (SEQ ID NO:54). One embodiment is an RSV immunogen that is resurfaced. One embodiment is an RSV immunogen that comprises an amino acid sequence of a protein selected from the group consisting of mota_{—}1lp 1b.m1.c1.d1_des1_{—}1 (SEQ ID NO:66), mota_{—}1lp1b.m1.c1.d1_des1_{—}2 (SEQ ID NO:67), mota_{—}1lp1b.m1.c1.d1_des1_{—}3 (SEQ ID NO:68), mota_{—}1lp1b.m1.c1.d1_des1_{—}5 (SEQ ID NO:69), mota_{—}1lp1b.m1.c1.d1_des1_{—}6 (SEQ ID NO:70), mota_{—}1lp1b.m1.c1.d1_des1_{—}7 (SEQ ID NO:71), mota_{—}1lp1b.m1.c1.d1_des1_{—}8 (SEQ ID NO:72), mota_{—}1lp1b.m1.c1.d1_des1_{—}9 (SEQ ID NO:73), mota_{—}1lp1b.m1.c1.d1_des1_{—}10 (SEQ ID NO:74), mota_{—}1lp1b.m1.c1.d1_des1_{—}11 (SEQ ID NO:75), 1lp1b_{—}003_Surf1 (SEQ ID NO:59), 1lp1b_{—}003_Surf6 (SEQ ID NO:62), and 1lp1b_{—}003_Surf8 (SEQ ID NO:65). One embodiment is an RSV immunogen that is a multivalent immunogen. One embodiment is an RSV immunogen that comprises an amino acid sequence of a protein selected from the group consisting of 1lp1b_{—}003 ferritin (SEQ ID NO:138), 1lp1b_{—}003_eumS (SEQ ID NO:140), 1lp1b_{—}003_eumSP (SEQ ID NO:142), 1lp1b_{—}003_eumL (SEQ ID NO:144), and 1lp1b_{—}003_eumLP (SEQ ID NO:146). One embodiment is an RSV immunogen that comprises an amino acid sequence of a protein selected from the group consisting of 1lp1b_{—}003_K46A (SEQ ID NO:78), 1lp1b_{—}003_Q52A (SEQ ID NO:81), 1lp1b_{—}003_I13L_F27A (SEQ ID NO:87), 1lp1b_{—}003_L41I_L42V (SEQ ID NO:90), 1lp1b-003_L41I_L42A (SEQ ID NO:93), lp1b_{—}003_I13A (SEQ ID NO:105), 1lp1b_{—}003_L16A (SEQ ID NO:108), 1lp1b_{—}003_F27A (SEQ ID NO:111), 1lp1b_{—}003_L41A (SEQ ID NO:114), 1lp1b_{—}003_L42A (SEQ ID NO:117), and 1lp1b_{—}003_Neg1 (SEQ ID NO:132). One embodiment is an immunogen comprising an amino acid sequence of a protein selected from the group consisting of 1lp1b_{—}003_C43 (SEQ ID NO:33), 1lp1b_{—}003_C47 (SEQ ID NO:36), 1lp1b_{—}003_Glyc3 (SEQ ID NO:45), 1lp1b_{—}003_Glyc4 (SEQ ID NO:48), 1lp1b_{—}003_Glyc5 (SEQ ID NO:51), 1lp1b_{—}003_Glyc6 (SEQ ID NO:54), 1lp1b_{—}003_K46A_Q52A (SEQ ID NO:84), lp1b_{—}003_Glycine1 (SEQ ID NO:120), 1lp1b_{—}003_Glycine2 (SEQ ID NO:123), 1lp1b_{—}003_Pos1 (SEQ ID NO:126), 1lp1b_{—}003_Pos2 (SEQ ID NO:129), and 1lp1b_{—}003_Neg2 (SEQ ID NO:135). One embodiment is an immunogen comprising an amino acid sequence of a protein selected from the group consisting of 1lp1b_{—}003_I13A_L42A (SEQ ID NO:96), 1lp1b_{—}003_L19A (SEQ ID NO:99), and 1lp1b_{—}003_L19A_L41I (SEQ ID NO:102). An RSV immunogen disclosed herein can elicit a humoral immune response against RSV. In one embodiment, such an RSV immunogen can elicit a humoral immune response against RSV, but does not elicit a cellular immune response. An RSV immunogen as disclosed herein can have a motavizumab antibody binding domain comprising less than 12 consecutive amino acids from a motavizumab antibody binding domain of RSV fusion protein. In one embodiment, an RSV immunogen has a motavizumab antibody-binding domain comprising less than 9 consecutive amino acids from a motavizumab antibody-binding domain of RSV fusion protein.

The disclosure provides an RSV immunogen comprising an amino acid sequence of truncated 1S2X_a (SEQ ID NO:13) having from one to twenty-five amino acid substitutions, wherein at least one amino acid substitution is selected from the group consisting of:

(a) substitution of a serine at amino acid position 92 in SEQ ID NO:13;

(b) substitution of a leucine at amino acid position 95 in SEQ ID NO:13;

(c) substitution of a serine at amino acid position 96 in SEQ ID NO:13;

(d) substitution of an isoleucine at amino acid position 98 in SEQ ID NO:13;

(e) substitution of an asparagine at amino acid position 99 in SEQ ID NO:13;

(f) substitution of an aspartic acid at amino acid position 100 in SEQ ID NO:13;

(g) substitution of an asparagine at amino acid position 105 in SEQ ID NO:13;

(h) substitution of an aspartic acid at amino acid position 106 in SEQ ID NO:13;

(i) substitution of a lysine at amino acid position 108 in SEQ ID NO:13;

(j) substitution of a lysine at amino acid position 109 in SEQ ID NO:13;

(k) substitution of a leucine at amino acid position 110 in SEQ ID NO:13;

(l) substitution of a serine at amino acid position 112 in SEQ ID NO:13; and

(m) substitution of an asparagine at amino acid position 113 in SEQ ID NO:13. Such an RSV immunogen can comprise all of the substitutions (a) through (m). An RSV immunogen can elicit a humoral immune response against RSV. The phrase “from one to twenty-five amino acid substitutions” allows for substitutions beyond those that are specified in (a) through (m). In one embodiment, the three-dimension structure of the antibody-binding domain is maintained or improved when such substitution(s) are made. One embodiment is an RSV immunogen that comprises an amino acid sequence that has up to twelve substitutions (i.e., any number ranging from 0 through 12 substitutions) in an amino acid sequence of a protein selected from the group consisting of 1s2xa_{—}001 (SEQ ID NO:152), 1s2xa_{—}002 (SEQ ID NO:155), 1s2xa_{—}003 (SEQ ID NO:158), and 1s2xa_{—}004 (SEQ ID NO:164). One embodiment is an RSV immunogen that comprises an amino acid sequence that has up to twelve substitutions in the amino acid sequence of protein 1s2xa_{—}003 (SEQ ID NO:158). One embodiment is an RSV immunogen that comprises an amino acid sequence that is at least 95% identical to an amino acid sequence of a protein selected from the group consisting of 1s2xa_{—}001 (SEQ ID NO:152), 1s2xa_{—}002 (SEQ ID NO:155), 1s2xa_{—}003 (SEQ ID NO:158), and 1s2xa_{—}004 (SEQ ID NO:164). One embodiment is an RSV immunogen that comprises an amino acid sequence that is at least 95% identical to an amino acid sequence of protein 1s2xa_{—}003 (SEQ ID NO:158). One embodiment is an RSV immunogen that comprises an amino acid sequence of a protein selected from the group consisting of 1s2xa_{—}001 (SEQ ID NO:152), 1s2xa_{—}002 (SEQ ID NO:155), 1s2xa_{—}003 (SEQ ID NO:158), and 1s2xa_{—}004 (SEQ ID NO:164). One embodiment is an RSV immunogen comprising 1s2xa_{—}003; such an immunogen comprises amino acid sequence SEQ ID NO:158. One embodiment is an RSV immunogen that is N-linked glycosylated. One embodiment is an RSV immunogen that is resurfaced. One embodiment is an RSV immunogen that is a multivalent immunogen. An RSV immunogen disclosed herein can elicit a humoral immune response against RSV. In one embodiment, such an RSV immunogen can elicit a humoral immune response against RSV, but does not elicit a cellular immune response. An RSV immunogen as disclosed herein can have a motavizumab antibody binding domain comprising less than 12 consecutive amino acids from a motavizumab antibody binding domain of RSV fusion protein. In one embodiment, an RSV immunogen has a motavizumab antibody-binding domain comprising less than 9 consecutive amino acids from a motavizumab antibody-binding domain of RSV fusion protein.

An immunogen of the embodiments does not have an amino acid sequence that consists of SEQ ID NO:1. An immunogen of the embodiments does not have an amino acid sequence that consists of SEQ ID NO:2. An immunogen of the embodiments does not have an amino acid sequence that consists of SEQ ID NO:3. An immunogen of the embodiments does not have an amino acid sequence that consists of SEQ ID NO:9. An immunogen of the embodiments does not have an amino acid sequence that consists of SEQ ID NO:10.

The present invention also discloses an immunogen comprising an RSV F protein that is stabilized in its pre-fusion, trimeric state. Such an immunogen comprises an RSV F protein in which the furin cleavage sites can (but need not be) mutated to reduce or prevent cleavage and a trimerization motif (such as a fibritin T4 trimerization motif) preferably appended to a truncated C terminus lacking the F protein transmembrane and cellular domain so that the resultant RSV F protein remains in a trimeric, pre-fusion conformation.

One embodiment of the present invention is an immunogen comprising an amino acid sequence of a protein selected from RSV F₀ Fd (also referred to as RSV F0 Fd) (SEQ ID NO:174), RSV F Fd (SEQ ID NO:175), and RSV F0 Fd GAG (SEQ ID NO:176), the amino acid sequences of which are disclosed in the Examples. One embodiment is an immunogen comprising RSV F₀ Fd (SEQ ID NO:174). One embodiment is an immunogen comprising RSV F Fd (SEQ ID NO:175). One embodiment is an immunogen comprising RSV F0 Fd GAG (SEQ ID NO:176). One embodiment is a variant of any of such immunogens, wherein the variant shares at least 95%, at least 96%, at least 97%, at least 98%, or at least 99% amino acid sequence identity with such immunogen. Preferably the variant retains contact residues of such immunogen.

Immunogens of the instant invention can be produced recombinantly or they can be produced synthetically. Also encompassed are immunogens that are combinations of recombinant and synthetic molecules. General methods for producing and isolating recombinant or synthetic proteins or peptides are known to those skilled in the art. It should be noted that, as used herein, an isolated, or biologically pure, molecule, is one that has been removed from its natural milieu. As such the terms isolated, biologically pure, and the like, do not necessarily reflect the extent to which the immunogen has been purified.

An immunogen of the embodiments can also comprise one or more motifs that can aid in purification of the immunogen, processing of the immunogen, and/or the immunogenicity of the immunogen. Examples include, but are not limited to, an HRV3C site, a caspase 3 site, a His tag, a Strep tag, MBP (maltose binding protein) or a functional fragment thereof, a factor Xa site, a TEV site, and a PADRE motif. A PADRE (Pan HLA DR-binding epitope peptide) motif has been shown to elicit T-cell help to stimulate a good antibody response; see, e.g., Alexander J, et al., 1994, Immunity 1, 751-761.

One embodiment is a protein comprising an amino acid sequence of an immunogen of the embodiments. Such a protein can be produced recombinantly or synthetically.

One embodiment of the present invention is a nucleic acid molecule that encodes an immunogen of the present disclosure. Such a nucleic acid molecule comprises a nucleic acid sequence that encodes an amino acid sequence of an immunogen of the embodiments. A nucleic acid molecule of the embodiments can include DNA, RNA, or derivatives of either DNA or RNA. A nucleic acid molecule can encode one or more immunogens of the embodiments. Nucleic acid molecules of the disclosure have been subjected to human manipulation. Such a nucleic acid molecule can be produced using recombinant DNA technology (e.g., polymerase chain reaction (PCR) amplification, cloning), chemical synthesis, or a combination of recombinant DNA technology and chemical synthesis. In one embodiment, a nucleic acid molecule, such as a nucleic acid molecule encoding a scaffold protein, can be modified by inserting, deleting, substituting, and/or inverting one or more nucleotides to yield a nucleic acid molecule that encodes an immunogen of the present invention. A nucleic acid molecule can also be modified to introduce codons that are better recognized by the system used to produce protein from a nucleic acid molecule of the disclosure.

One embodiment is a nucleic acid molecule encoding an immunogen comprising a scaffold protein with one or more contact residues, as described herein, embedded in it. Such embedding can be accomplished using techniques described herein as well as techniques known by one skilled in the art.

Nucleic acid molecules of the present invention can be produced using a number of methods known to those skilled in the art; see, for example, Sambrook J et al., 2001, Molecular Cloning: a Laboratory Manual, 3^{rd} edition, Cold Spring Harbor Laboratory Press, and Ausubel F et al., 1994, Current Protocols in Molecular Biology, John Wiley & Sons. For example, nucleic acid molecules can be modified using a variety of techniques including, but not limited to, classic mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules and combinations thereof. Nucleic acid molecules of the embodiments can be selected from a mixture of modified nucleic acids by screening for the function of the protein encoded by the nucleic acid (e.g., the ability of such a nucleic acid molecule to encode an immunogen that binds to motavizumab or 101F).

The disclosure provides a recombinant molecule that comprises a nucleic acid encoding an immunogen of the embodiments operatively linked to at least one transcription control sequence capable of effecting expression of the nucleic acid molecule in a recombinant cell. A recombinant cell is a host cell that is transformed with a recombinant molecule of the embodiments; i.e., a recombinant cell comprises a recombinant molecule. A recombinant molecule can comprise one or more nucleic acid molecules encoding an immunogen of the embodiments operatively linked to one or more transcription control sequences. A recombinant cell can comprise one or more recombinant molecules. In one embodiment, a nucleic acid molecule is operatively linked to a recombinant vector that includes a transcription control sequence to produce a recombinant molecule. Such a vector can be a plasmid vector, a viral vector, or other vector. Such a vector can be DNA, RNA, or a derivative of DNA or RNA. Host cells to transform can be selected based on their ability to effect expression of a nucleic acid molecule of the embodiments. Host cells can also be selected that effect post-translational modifications. Methods to select, produce and use recombinant vectors, recombinant molecules, and recombinant cells of the embodiments are known to those skilled in the art. Proteins and immunogens of the embodiments can be produced by culturing recombinant cells of the embodiments. Methods to effect such production and recovery of such proteins and immunogens are known to those skilled in the art, see for example Sambrook J et al., ibid, and Ausubel, F et al., ibid.

The disclosure also provides a recombinant molecule that is a nucleic acid immunogen or vaccine. That is, such a recombinant molecule can be administered to a subject to elicit a humoral immune response against RSV. Such a response can be a neutralizing humoral immune response. Such a response can be protective. Such a vaccine comprises a recombinant molecule comprising a nucleic acid molecule that encodes an immunogen of the embodiments. In one embodiment, the recombinant molecule is a nucleic acid molecule of the embodiments operatively linked to a recombinant vector. Suitable vectors can be selected by one skilled in the art. Examples include, but are not limited, to adenovirus, adeno-associated virus, cytomegalovirus (CMV), herpes virus, poliovirus, retrovirus, sindbis virus, vaccinia virus, or any other DNA or RNA virus vector.

The present invention also discloses methods of making an immunogen of the present invention. One embodiment is a method that involves using the three-dimensional structure of the antibody-binding domain of a RSV F peptide, when such peptide is bound to an RSV neutralizing antibody, to identify a protein comprising a similar three-dimensional structure, and then substituting the contact residues from the RSV F peptide into the spatially corresponding positions in the native scaffold protein to create an immunogen. In one embodiment, the RSV-neutralizing antibody is palivizumab (SYNAGIS®). In another embodiment, the RSV-neutralizing antibody is motavizumab. In yet another embodiment, the RSV-neutralizing antibody is 101F. In one embodiment the three-dimensional structure of complex between the RSV F peptide and the antibody is represented by atomic coordinates defining backbone atoms that have a root mean square deviation of less than 10 angstroms from the backbone atoms of the complex defined by the coordinates recited in PDB acc code 3IXT or in PDB acc code 3O41. A preferred embodiment of the present invention is a method to produce an immunogen that elicits a potent neutralizing humoral response against RSV, the method comprising:

(a) obtaining a three-dimensional model substantially corresponding to the three-dimensional model represented by the coordinates set forth in PDB acc code 3IXT or in PDB acc code 3O41;

(b) using the model obtained in (a) model to identify a native protein scaffold having a three-dimensional structure represented by atomic coordinates that have a root mean square deviation of protein backbone atoms of less than 10 angstroms when superimposed on the three-dimensional model of the RSV F peptide defined by atomic coordinates in PDB acc code 3IXT or in PDB acc code 3O41;

(c) substituting amino acids in the native protein scaffold that spatially correspond to contact residues in the RSV F protein in such three-dimensional model, with the spatially corresponding contact residues in the F protein in such three-dimensional model to create an immunogen containing a transplanted epitope; and

(d) producing said immunogen comprising such transplanted epitope. As has previously been discussed, immunogens produced using the disclosed methods can also be modified to remove sequences related to the RSV F protein, reduce steric hindrance and/or to increase the affinity of the immunogen for motavizumab or 101F. Thus, in one embodiment, the method further comprises modifying the immunogen created in step (c) by substituting amino acids outside of the antibody-binding domain to (a) reduce steric hindrance, (b) introduce new ionic bonds between the immunogen and the antibody, (c) stabilize the protein in a conformation that maintains the transplanted epitope in the spatial conformation found in the three-dimensional model represented by the coordinates in PDB acc code 3IXT or in PDB acc code 3O41. Such a method can also include, but not be limited to, introducing flexibility, N-linked glycosylation sites, positively or negatively charged amino acids, shielding against immunodominant epitopes, or other beneficial features.

The disclosure provides a method to produce an immunogen that elicits a potent neutralizing humoral immune response against RSV. The method comprises:

(a) obtaining a three-dimensional model substantially corresponding to the three-dimensional model of a complex between an RSV F peptide consisting of amino acid sequence SEQ ID NO:2 and motavizumab, wherein the model substantially represents the atomic coordinates specified in Protein Data Bank accession code 3IXT;

(b) using the model to identify a candidate protein scaffold having a three-dimensional structure represented by atomic coordinates that have a root mean square deviation of protein backbone atoms of less than 10 angstroms when superimposed on the three-dimensional model of the RSV F peptide defined by atomic coordinates in Protein Data Bank accession code 3IXT;

(c) substituting amino acids in the candidate protein scaffold that spatially correspond to contact residues in the RSV F protein in the three-dimensional model, with the spatially corresponding contact residues in the F protein in the three-dimensional model to create a protein containing a transplanted epitope; and

(d) producing the protein comprising the transplanted epitope. The method can further include the step of modifying the protein of step (c) to stabilize the protein in a conformation that maintains the transplanted epitope in the antibody-bound conformation of the three-dimensional model of a complex between an RSV F peptide consisting of amino acid sequence SEQ ID NO:2 and motavizumab, wherein the model substantially represents the atomic coordinates specified in Protein Data Bank accession code 3IXT.

The disclosure also provides a method to produce an immunogen that elicits a potent neutralizing humoral immune response against RSV. The method comprises:

(a) obtaining a three-dimensional model substantially corresponding to the three-dimensional model of a complex between an RSV F peptide consisting of amino acid sequence SEQ ID NO:4 and 101F antibody, wherein the model substantially represents the atomic coordinates specified in Protein Data Bank accession code 3O41;

(b) using the model to identify a candidate protein scaffold having a three-dimensional structure represented by atomic coordinates that have a root mean square deviation of protein backbone atoms of less than 10 angstroms when superimposed on the three-dimensional model of the RSV F peptide defined by atomic coordinates in Protein Data Bank accession code 3O41;

(c) substituting amino acids in the candidate protein scaffold that spatially correspond to contact residues in the RSV F protein in the three-dimensional model, with the spatially corresponding contact residues in the F protein in the three-dimensional model to create a protein containing transplanted epitope; and

(d) producing the protein comprising the transplanted epitope. The method can further include the step of modifying the protein of step (c) to stabilize the protein in a conformation that maintains the transplanted epitope in the antibody-bound conformation of the three-dimensional model of a complex between an RSV F peptide consisting of amino acid sequence SEQ ID NO:4 and 101F antibody, wherein the model substantially represents the atomic coordinates specified in Protein Data Bank accession code 3O41.

The three-dimensional model of a complex between the RSV F peptide and motavizumab, or 101F, disclosed herein provides an understanding of how residues in each molecule interact to form a complex. As disclosed herein, such information is useful in producing immunogens that stimulate a humoral immune response against the RSV F protein. Such information can also be used to produce an antibody (also referred to herein as an antibody protein) that has a higher, or lower, affinity for the RSV F peptide. More specifically, by knowing how the peptide and the antibody align in three-dimensional space, the sequence of the antibody can be altered to introduce new amino acids capable of forming bonds with amino acids in the peptide. Thus, one embodiment of the present invention is a modified RSV neutralizing antibody that is more potent than motavizumab, or 101F; such modified antibody comprises a peptide-binding site for the RSV F peptide, wherein such modified antibody contains amino acid substitutions when compared to the amino acid sequence of motavizumab or 101F, wherein such substitutions result in the formation of new ionic bonds between the modified antibody and the RSV F peptide, and wherein such new ionic bonds result in the modified antibody having a higher affinity for the RSV F protein. In one embodiment, the modified antibody is created by introducing sequence alterations into the amino acid sequence of motavizumab. In another embodiment, the modified antibody is created by introducing sequence alterations into the amino acid sequence of 101F. An antibody protein of the embodiments can be of any size that exhibits more potent neutralization of RSV than does motavizumab or 101F antibody. For example, an antibody protein can comprise an entire heavy chain and an entire light chain or can comprise a portion thereof that retains more potent neutralization activity. In one embodiment an antibody protein is an antigen-binding fragment. In one embodiment, an antibody protein is a single polypeptide chain.

A preferred embodiment of the present invention is a modified neutralizing antibody that exhibits more potent neutralization of RSV than does motavizumab, wherein such modified antibody is produced by:

(a) obtaining a three-dimensional model of a complex between an RSV F peptide consisting of amino acid sequence SEQ ID NO:2 and motavizumab, such complex being set forth in the three-dimensional model defined by the atomic coordinates in PDB acc code 3IXT;

(b) identifying at least one amino acid change in the interface between motavizumab and the RSV F protein, wherein such at least one change, if incorporated into motavizumab, would yield an antibody protein with a higher affinity for RSV F protein; and

(c) producing such antibody protein comprising such at least one change. Another embodiment is a modified neutralizing antibody that exhibits more potent neutralization of RSV than does 101F antibody, wherein such modified antibody is produced by:

(a) obtaining a three-dimensional model of a complex between an RSV F peptide consisting of amino acid sequence SEQ ID NO:4 and 101F antibody, the complex being set forth in the three-dimensional model defined by the atomic coordinates in PDB acc code 3O41;

(b) identifying at least one amino acid change in the interface between 101F antibody and the RSV F protein, wherein such at least one change, if incorporated into 101F antibody, would yield an antibody protein with a higher affinity for RSV F protein; and

(c) producing such antibody protein comprising such at least one change.

As used herein, an antibody protein that exhibits more potent neutralization of RSV than does motavizumab means that a lower titer of such antibody protein is required to neutralize a given amount of RSV, as compared to the titer of motavizumab required to neutralize the same amount of RSV. As used herein, an antibody that exhibits more potent neutralization of RSV than does 101F antibody means that a lower titer of such antibody is required to neutralize a given amount of RSV, as compared to the titer of 101F required to neutralize the same amount of RSV. Suitable amino acid changes to the sequence of motavizumab that result in an antibody protein having a higher affinity for the RSV F protein are disclosed herein. This technique can also be used to modify other antibodies that bind to the motavizumab-binding site or the 101F antibody-binding site of RSV F protein.

One embodiment of the present invention is a modified antibody, wherein the heavy chain of such antibody comprises SEQ ID NO:5, except that such heavy chain comprises at least one amino acid substitution selected from the group consisting of:

(a) the amino acid at position 32 of SEQ ID NO:5 is substituted with a histidine or a glutamic acid;

(b) the amino acid at position 35 of SEQ ID NO:5 is substituted with an alanine;

(c) the amino acid at position 52 of SEQ ID NO:5 is substituted with an amino acid selected from the group consisting of lysine, histidine, threonine, serine and arginine;

(d) the amino acid at position 53 of SEQ ID NO:5 is substituted with a histidine or a serine;

(e) the amino acid at position 54 of SEQ ID NO:5 is substituted with a phenylalanine or an arginine;

(f) the amino acid at position 56 of SEQ ID NO:5 is substituted with an amino acid selected from the group consisting of isoleucine, serine, glutamic acid, and aspartic acid;

(g) the amino acid at position 58 of SEQ ID NO:5 is substituted with a tyrosine or a tryptophan;

(h) the amino acid at position 97 of SEQ ID NO:5 is substituted with an aspartic acid, a histidine or an arginine;

(i) the amino acid at position 99 of SEQ ID NO:5 is substituted with aspartic acid;

(j) the amino acid at position 100 of SEQ ID NO:5 is substituted with a tyrosine, a tryptophan or a histidine; and

(k) the amino acid at position 100A of SEQ ID NO:5 is substituted with a serine or a threonine.

Another embodiment of the present invention is a modified antibody, wherein the light chain of such antibody comprises SEQ ID NO:6, except that such light chain comprises at least one amino acid substitution selected from the group consisting of:

(a) the amino acid at position 32 of SEQ ID NO:6 is substituted with a phenylalanine;

(b) the amino acid at position 49 of SEQ ID NO:6 is substituted with an histidine or an arginine;

(c) the amino acid at position 92 of SEQ ID NO:6 is substituted with lysine;

(d) the amino acid at position 94 of SEQ ID NO:6 is substituted with a histidine; and,

(e) the amino acid at position 96 of SEQ ID NO:6 is substituted with a histidine.

In addition to the substitutions described above, analysis of the three-dimensional model of the complex between the RSV F peptide and motavizumab, or 101F, disclosed herein, indicates that additional contacts between the antibody and the peptide can be made by increasing the length of the CDRH2 loop in the antibody (which spans amino acids 50 through 58 of the heavy chain) by 2 residues. One embodiment of the present invention is a modified antibody, wherein the heavy chain of such antibody comprises SEQ ID NO:5, except that amino acids 50 through 58 of SEQ ID NO:5 have been replaced with an 11 amino acid sequence defined as follows:

(a) position one of said 11 amino acid sequence is a glutamic acid, a serine, or a methionine;

(b) position two of said 11 amino acid sequence is an isoleucine;

(c) position three of said 11 amino acid sequence is a histidine, an arginine, or a phenylalanine;

(d) position four of said 11 amino acid sequence is a serine;

(e) position five of said 11 amino acid sequence is a glycine;

(f) position six of said 11 amino acid sequence is an amino acid selected from the group consisting of glycine, histidine, lysine, leucine, asparagine, glutamine, serine, aspartic acid, threonine, and arginine;

(g) position seven of said 11 amino acid sequence is an amino acid selected from the group consisting of phenylalanine, lysine, serine, threonine, aspartic acid, and arginine;

(h) position eight of said 11 amino acid sequence is a glutamic acid, an asparagine, or an aspartic acid;

(i) position nine of said 11 amino acid sequence is an amino acid selected from the group consisting of aspartic acid, histidine, leucine, serine, arginine, and threonine;

(j) position ten of said 11 amino acid sequence is a tyrosine; and

(k) position eleven of said 11 amino acid sequence is a tyrosine, a phenylalanine or a histidine.

It should be understood that any combination of the above-described substitutions can be made. That is, in addition to substituting the eleven amino acid sequence described above, other substitutions can be made outside of amino acids 50-58 of SEQ ID NO:5, (e.g., substitutions into positions 32, 35, 97, 99, 100 or 100A of SEQ ID NO:5, and/or substitutions in to the light chain), so long as the resultant antibody exhibits more potent neutralization of RSV than does motavizumab or 101F.

The disclosure provides a protein comprising an amino acid sequence of any of the antibody proteins of the embodiments. The disclosure also provides a nucleic acid molecule encoding any of the antibody proteins of the embodiments. Such a nucleic acid molecule can encode one or more antibody proteins. The disclosure further provides a recombinant molecule that comprises a nucleic acid molecule encoding an antibody protein of the embodiments operatively linked to at least one transcription control sequence capable of effecting expression of the nucleic acid molecule in a recombinant cell. A recombinant molecule can comprise one or more nucleic acid molecules encoding an antibody protein of the embodiments operatively linked to one or more transcription control sequences. The disclosure also provides a recombinant cell transformed with a recombinant molecule of the embodiments; i.e., a recombinant cell comprises a recombinant molecule. A recombinant cell can comprise one or more recombinant molecules.

The disclosure provides methods to produce antibody proteins of the embodiments. An antibody protein can be produced synthetically, recombinantly, or by a combination of synthetic and recombinant methods. Methods such as those taught herein for production of immunogens can be used. In addition, methods are known to those skilled in the art.

The disclosure provides a method to produce a composition comprising a neutralizing antibody protein that exhibits more potent neutralization of RSV than does motavizumab. The method comprises:

(a) obtaining a three-dimensional model of a complex between an RSV F peptide consisting of amino acid sequence SEQ ID NO:2 and motavizumab, the complex being set forth in the 3-dimensional model defined by the atomic coordinates in Protein Data Bank accession code 3IXT;

(b) identifying at least one amino acid change in the interface between motavizumab and the RSV F protein, wherein the at least one change, if incorporated into motavizumab, would yield an antibody protein with a higher affinity for RSV F protein; and

(c) producing the antibody protein comprising the at least one change.

The disclosure also provides a method to produce a composition comprising a neutralizing antibody protein that exhibits more potent neutralization of RSV than does 101F antibody. The method comprises:

(a) obtaining a three-dimensional model of a complex between an RSV F peptide consisting of amino acid sequence SEQ ID NO:4 and 101F antibody, the complex being set forth in the 3-dimensional model defined by the atomic coordinates in Protein Data Bank accession code 3O41;

(b) identifying at least one amino acid change in the interface between the 101F antibody and the RSV F protein, wherein the at least one change, if incorporated into the 101F antibody, would yield an antibody protein with a higher affinity for RSV F protein; and

(c) producing the antibody protein comprising the at least one change.

One embodiment of the present invention is an isolated crystal of a complex between an RSV F peptide and motavizumab. Another embodiment of the present invention is an isolated crystal of a complex between an RSV F peptide and 101F. As used herein, an isolated crystal is a crystal of a protein, or complex of proteins, that has been produced in a laboratory; that is, an isolated crystal is produced by an individual and is not an object found in situ in nature. It is appreciated by those skilled in the art that there are a variety of techniques to produce crystals including, but not limited to, vapor diffusion using a hanging or sitting drop methodology, vapor diffusion under oil, and batch methods; see, for example, Ducruix et al., eds., 1991, Crystallization of nucleic acids and proteins; A practical approach, Oxford University Press, and Wyckoff et al., eds., 1985, Methods in Enzymology 11, 49-185; each reference is incorporated by reference herein in its entirety. It is also to be appreciated that crystallization conditions can be adjusted depending on a protein's inherent characteristics as well as on a protein's concentration in a solution and that a variety of precipitants can be added to a protein solution in order to effect crystallization; such precipitants are known to those skilled in the art. In a preferred embodiment, a crystal of a complex between an RSV F peptide and motavizumab or 101F is produced in a solution by adding a precipitant such as polyethylene glycol (PEG) or PEG monomethylether.

One embodiment of the present invention is an isolated crystal of a complex between an RSV F peptide and motavizumab, or 101F, such crystal being produced by the vapor diffusion method using a reservoir solution comprising about 17.5% (w/v) PEG 8000, 0.2 M zinc acetate, and 0.1 M cacodylate pH 6.5. Another embodiment of the present invention is an isolated crystal of a complex between an RSV F peptide and motavizumab, or 101F, wherein obtained by a method comprising:
(a) producing an initial crystal using the vapor diffusion method at a temperature of about 20° C., with a reservoir solution comprising about 17.5% (w/v) PEG 8000, 0.2 M zinc acetate, and 0.1 M cacodylate pH 6.5; and
(b) streak-seeding the initial crystal obtained in (a) into hanging drops consisting of 1 μl of protein complex and 1 μl of 30% (w/v) PEG 1500.

Isolated crystals of the present invention can include heavy atom derivatives, such as, but not limited to, gold, platinum, mercury, selenium, copper, and lead. Such heavy atoms can be introduced randomly or introduced in a manner based on knowledge of three-dimensional models of the present invention. Additional crystals of the present invention are not derivatized.

A preferred crystal of the present invention diffracts X-rays to a resolution of about 4.5 angstroms or higher (i.e., lower number meaning higher resolution), with resolutions of about 4.0 angstroms or higher, about 3.5 angstroms or higher, about 3.25 angstroms or higher, about 3 angstroms or higher, about 2.5 angstroms or higher, about 2.3 angstroms or higher, about 2 angstroms or higher, about 1.5 angstroms or higher, and about 1 angstrom or higher being increasingly more preferred. It is appreciated, however, that additional crystals of lower resolutions can have utility in discerning overall topology of the structures, e.g., location of a contact residues between an F peptide and its respective antibody. Preferred are crystals are those described in Table 3 and Table 4.

TABLE 3
Data collection and refinement statistics (molecular replacement)

| | Motavizumab/peptide |
|---|---|
| Data collection | |
| Space group | P4₃2₁2 |
| Cell dimensions | |
| a = b, c (Å) | 90.75, 232.06 |
| Resolution (Å) | 50-2.75 |
| R_{merge} | 11.3 (52.9) |
| I/σI | 12.2 (1.8) |
| Completeness (%) | 93.9 (88.3) |
| Redundancy | 4.6 (3.5) |
| Refinement | |
| Resolution (Å) | 2.75 |
| No. reflections | 23,502 |
| Rwork/Rfree (%) | 21.3/27.4 |
| No. atoms | |
| Protein | 6464 |
| Ligand/ion | 396 |
| Water | 150 |
| B-factors | |
| Protein | 57.1 |
| Ligand/ion | 89.2 |
| Water | 47.0 |
| R.m.s. deviations | |
| Bond lengths (Å) | 0.004 |
| Bond angles (°) | 0.742 |

Values in parentheses are for highest-resolution shell.

TABLE 4
Data collection and refinement statistics (molecular replacement)

| | 101F/peptide |
|---|---|
| Data collection | |
| Space group | P2₁2₁2₁ |
| Cell dimensions | |
| a, b, c (Å) | 79.90, 92.98, 141.22 |
| Resolution (Å) | 50-1.95 |
| R_{merge} | 10.7 (47.9) |
| I/σI | 15.5 (2.0) |
| Completeness (%) | 95.7 (77.4) |
| Redundancy | 5.9 (4.3) |
| Refinement | |
| Resolution (Å) | 1.95 |
| No. reflections | 69,877 |
| Rwork/Rfree (%) | 17.7/22.0 |
| No. atoms | |
| Protein | 6695 |
| Ligand/ion | 223 |
| Water | 727 |
| B-factors | |
| Protein | 43.1 |
| Ligand/ion | 61.2 |
| Water | 50.7 |
| R.m.s. deviations | |
| Bond lengths (Å) | 0.006 |
| Bond angles (°) | 1.042 |

Values in parentheses are for highest-resolution shell.

One embodiment of the present invention is a three-dimensional model of a complex between an RSV F peptide consisting of SEQ ID NO:2 and motavizumab, wherein said model is substantially represented by the atomic coordinates specified in PDB acc code 3IXT. Another embodiment of the present invention is a three-dimensional model of a complex between an RSV F peptide consisting of SEQ ID NO:4 and 101F, wherein said model is substantially represented by the atomic coordinates specified in PDB acc code 3O41. Another embodiment of the present invention is a three-dimensional model of a complex between an RSV F peptide consisting of SEQ ID NO:9 and 101F, wherein the model is substantially represented by the atomic coordinates specified in PDB acc code 3O45. As used herein, a model that is substantially represented by atomic coordinates listed herein includes not only those models literally represented by the coordinates but also models representing a coordinate transformation of atomic coordinates disclosed herein, for example, by changing the relative spatial orientation of the coordinates. A three-dimensional model of a complex between an RSV F peptide and motavizumab, or 101F, is a representation, a mathematical model, or image that predicts the actual structure of the corresponding complex. As such, a three-dimensional model is a tool that can be used to probe the relationship between the region's structure and function at the atomic level and to design immunogens and modified. It is well known to those skilled in the art, however, that a three-dimensional model of a protein derived by analysis of protein crystals is not identical to the inherent structure of the protein. See, for example, Branden et al., Introduction to Protein Structure, Garland Publishing Inc., New York and London, 1991, especially on page 277, which states “not surprisingly the model never corresponds precisely to the actual crystal.” Furthermore, the model can be subjected to further refinements to more closely correspond to the actual structure of a complex between an RSV F peptide and motavizumab or 101F. Such a refined model, which is an example of a modification of the present invention, is a better predictor of the actual structure and mechanism of action of the protein that the model represents. Refinements can include models determined to more preferred degrees of resolution, preferably to about 4.5 angstroms, more preferably to about 4 angstroms, more preferably to about 3.5 angstroms, more preferably to about 3.25 angstroms, more preferably to about 3 angstroms, more preferably to about 2.5 angstroms, more preferably to about 2.3 angstroms, more preferably to about 2 angstroms, more preferably to about 1.5 angstroms, and even more preferably to about 1 angstrom. Preferred refinements are obtained using the three-dimensional model as a basis for such improvements.

One embodiment of the present invention is a composition comprising an immunogen or an antibody protein of the present invention. Another embodiment is a composition comprising a nucleic acid molecule, protein, recombinant molecule or recombinant cell of the embodiments. One type of composition is a vaccine. A composition of the present invention can be formulated in an excipient that a patient to be treated can tolerate. Examples of such excipients include water, saline, Ringer's solution, dextrose solution, Hank's solution, and other aqueous physiologically balanced salt solutions. Excipients can also contain minor amounts of additives, such as substances that enhance isotonicity and chemical or biological stability. Examples of buffers include phosphate buffer, bicarbonate buffer, and Tris buffer. Standard formulations can either be liquids or solids that can be taken up in a suitable liquid as a suspension or solution for administration to a patient. In one embodiment, a non-liquid formulation may comprise the excipient salts, buffers, stabilizers, etc., to which sterile water or saline can be added prior to administration.

A composition of the present invention may also include one or more adjuvants or carriers. Adjuvants are typically substances that enhance the immune response of a patient to a specific antigen, and carriers include those compounds that increase the half-life of a composition in the treated patient.

Immunogens and antibodies of the present invention are intended for use in protection against infection by RSV. The immunogens disclosed herein protect against RSV infection by eliciting a humoral immune response against the F protein of RSV. This humoral response results in neutralization of the virus. Antibodies of the present invention protect against infection with RSV by binding and neutralizing the virus. Thus one embodiment of the present invention is a method to protect a patient from RSV infection, the method comprising administering to the patient an immunogen or an antibody produced using the methods disclosed herein. One embodiment is a method to elicit a neutralizing humoral immune response against RSV, the method comprising administering an immunogen of the embodiments, wherein such administration elicits a neutralizing humoral immune response against RSV. In one embodiment, the immunogen is administered to a patient. One embodiment is a method to protect a patient from RSV infection comprising administering to the patient an immunogen, wherein such administration protects the patient from RSV infection. One embodiment is a method to elicit a neutralizing humoral immune response against RSV, the method comprising administering a nucleic acid vaccine of the embodiments, wherein such administration elicits a neutralizing humoral immune response against RSV. In one embodiment, the nucleic acid vaccine is administered to a patient. One embodiment is a method to protect a patient from RSV infection comprising administering to the patient a nucleic acid vaccine, wherein such administration protects the patient from RSV infection. One embodiment is a method to protect a patient from RSV infection comprising administering to the patient an antibody protein, wherein such administration protects the patient from RSV infection.

As used herein the phrase protect a patient from RSV infection includes preventing a patient from being infected by RSV, as well as treating a patient already infected with RSV. As used herein the term patient refers to any animal in need of such prevention or treatment. The animal can be a human or a non-human animal. A preferred animal to treat is a mammal. A patient can be of any age. In one embodiment, an immunogen or antibody can be administered to an infant. In one embodiment, an immunogen or antibody can be administered to a patient that is older than an infant. An immunogen or antibody can be administered or applied per se, or as a composition. An immunogen or antibody of the present invention, or a composition thereof, can be administered to a patient by a variety of routes, including, but limited to, by injection (e.g., intravenous, intramuscular, subcutaneous, intrathecal, intraperitoneal), by inhalation, by oral (e.g., in a pill, tablet, capsule, powder, syrup, solution, suspension, thin film, dispersion or emulsion.), transdermal, transmucosal, pulmonary, buccal, intranasal, sublingual, intracerebral, intravaginal rectal or topical administration or by any other convenient method known to those of skill in the art.

The amount of an immunogen or antibody of the present invention, and/or a composition thereof that will be effective can be determined by standard clinical techniques known in the art. Such an amount is dependent on, among other factors, the patient being treated, including, but not limited to the weight, age, and condition of the patient, the intended effect of the composition, the manner of administration and the judgment of the prescribing physician.

An immunogen or antibody of the present invention, or a composition thereof, can be administered alone or in combination with one or more other pharmaceutical agents, including other immunogens or antibodies of the present invention. The specific composition depends on the desired mode of administration, as is well known to the skilled artisan. One composition can include an immunogen of the present invention comprising motavizumab-binding contact residues. Another composition can include an immunogen of the present invention comprising 101F-binding contact residues. One composition comprises a combination of both immunogens. Another composition is an antibody of the present invention. Yet another composition comprises a nucleic acid vaccine comprising at least one nucleic acid molecule encoding an immunogen of the present invention. The disclosure also provides for a combination comprising one or more immunogens and/or antibodies (i.e., antibody proteins) of the embodiments with one or more other RSV immunogens and/or antibodies. The disclosure also provides for a combination comprising one or more immunogens and/or antibodies (i.e., antibody proteins) of the embodiments with one or more protective agents, such as, but not limited to, an agent that protects from infection by a virus, bacterium, parasite, or other infectious agents.

In one embodiment, administration can comprise a prime followed by one or more boosts. A prime can comprise a composition comprising at least one of the immunogens disclosed herein, or a nucleic acid encoding such an immunogen. A boost can comprise at least one of the immunogen disclosed herein, or a nucleic acid encoding such an immunogen. In one embodiment the boost comprises an immunogen that has been resurfaced (compared to the first immunogen) to further boost the humoral immune response against RSV contact residues in the motavizumab or 101F binding domains. In one embodiment the boost comprises a multivalent immunogen.

### EXAMPLES

The following examples are put forth so as to provide those of ordinary skill in the art with a complete disclosure and description of how to make and use the embodiments, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, and temperature is in degrees Celsius. Standard abbreviations may be used. For example, amino acids can be denoted by either the standard 3-letter or 1-letter code.

### Example 1

### Three-Dimensional Structure of Rsv F Protein and Motavizumab

This Example describes the crystallization and determination of the 3-dimensional structure of a complex between motavizumab and the 24-residue RSV fusion (F) peptide spanning amino acids 254-277 of the F protein (i.e., NSELLSLIND MPITNDQKKL MSNN, also denoted herein as SEQ ID NO:2) that includes the binding domain of motavizumab. The amino acid sequence of the F protein used in these studies is as follows: MELLILKANA ITTILTAVTF CFASGQNITE EFYQSTCSAV SKGYLSALRT GWYTSVITIE LSNIKENKCN GTDAKVKLIK QELDKYKNAV TELQLLMQST PATNNRARRE LPRFMNYTLN NAKKTNVTLS KKRKRRFLGF LLGVGSAIAS GVAVSKVLHL EGEVNKIKSA LLSTNKAVVS LSNGVSVLTS KVLDLKNYID KQLLPIVNKQ SCSISNIETV IEFQQKNNRL LEITREFSVN AGVTTPVSTY MLTNSELLSL INDMPITNDQ KKLMSNNVQI VRQQSYSIMS IIKEEVLAYV VQLPLYGVID TPCWKLHTSP LCTTNTKEGS NICLTRTDRG WYCDNAGSVS FFPQAETCKV QSNRVFCDTM NSLTLPSEVN LCNVDIFNPK YDCKIMTSKT DVSSSVITSL GAIVSCYGKT KCTASNKNRG IIKTFSNGCD YVSNKGVDTV SVGNTLYYVN KQEGKSLYVK GEPIINFYDP LVFPSDEFDA SISQVNEKIN QSLAFIRKSD ELL, also denoted herein as SEQ ID NO:1.

To enhance the potency of palivizumab, each residue in the six complementarity-determining regions (CDRs) was individually substituted with the other 19 amino acids (a total number of 1,121 unique single variants were assayed), and combinations of beneficial substitutions assessed (Wu H et al., 2007, J. Mol. Biol. 368, 652-665; Wu H et al., 2005, J. Mol. Biol. 350, 126-144). This led to the development of a second-generation antibody, motavizumab, which is ˜10 times more potent than palivizumab (Wu H et al., 2007, ibid.). Only 13 amino acids differ between motavizumab and palivizumab. Of these, seven individually increase the affinity of the antibody to the F glycoprotein, resulting in a 0.035 nM K_{d} (versus 1.4 nM for palivizumab) (Johnson S et al., 1997, J. Infect. Dis. 176, 1215-1224; Wu H et al., 2007, ibid.; Wu H et al., 2005, ibid.). This disclosure includes a characterization of the structural basis of motavizumab affinity, model mutations with enhanced affinity, and structural implications for motavizumab binding in the trimeric F glycoprotein context.

Motavizumab is ˜10-fold more potent than its predecessor, palivizumab (SYNAGIS®), the FDA-approved monoclonal antibody used to prevent respiratory syncytial virus (RSV) infection. The structure of motavizumab in complex with a 24-residue peptide corresponding to its epitope on the RSV-fusion (F) glycoprotein reveals the structural basis for its increased potency. Modeling suggests that motavizumab recognizes a different quaternary configuration of the F glycoprotein than observed in a homologous structure.

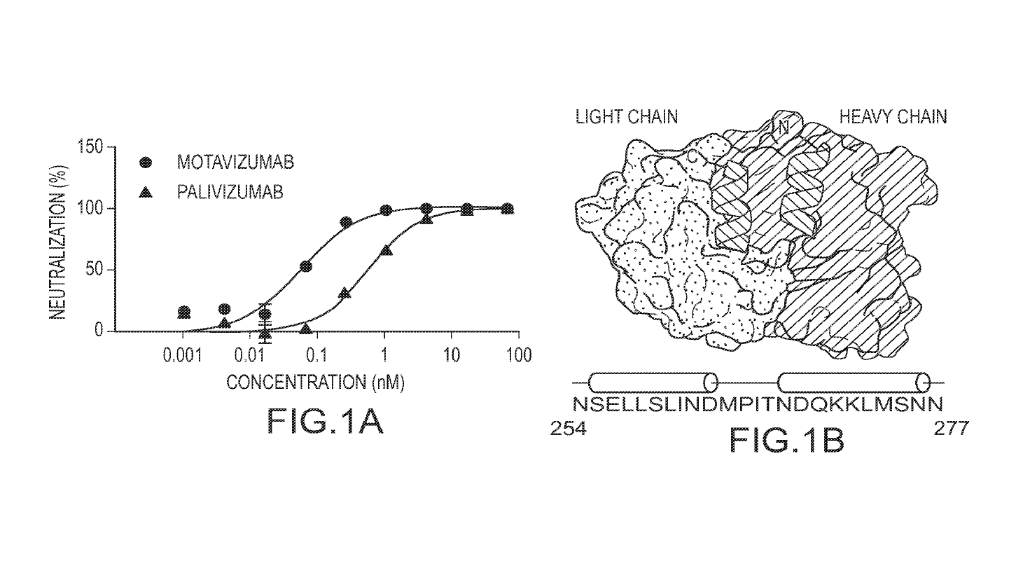

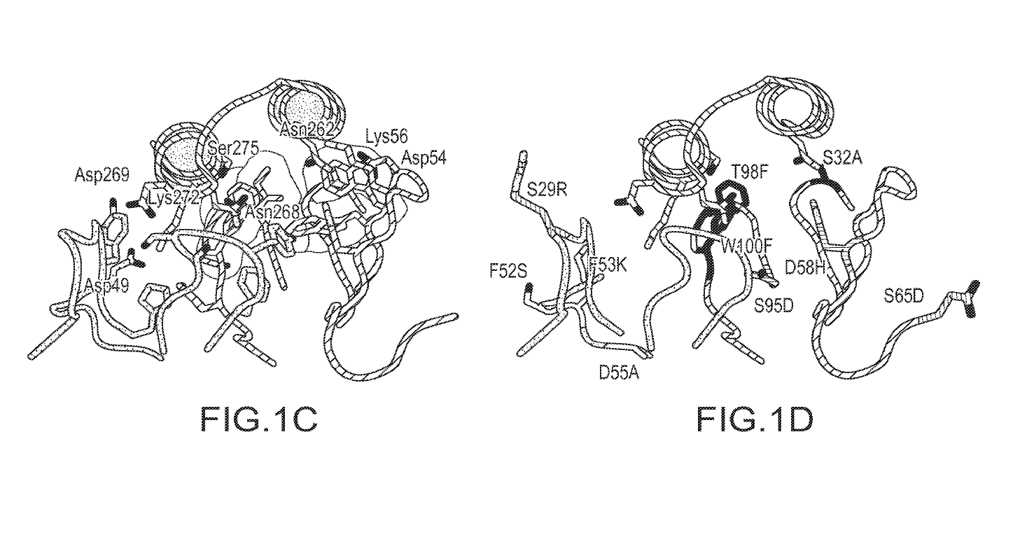

Recombinant motavizumab IgG molecules that were shown to neutralize RSV potently (FIG. 1a) were used to create antigen-binding fragments (Fabs) for crystallographic analysis. Crystals of the Fab were obtained in complex with a 24-residue peptide, which corresponds to residues 254-277 of the RSV F glycoprotein A2 strain (NSELLSLIND MPITNDQKKL MSNN) and represents the known epitope for palivizumab/motavizumab (Arbiza J et al., 1992, J. Gen. Virol. 74, 2225-2234). The crystals diffracted X-rays to 2.75 Å, and a molecular replacement solution was obtained containing two molecules of the previously-determined unliganded palivizumab structure (Johnson L S et al., U.S. Pat. No. 7,229,618, issued Jun. 12, 2007) per asymmetric unit. Initial maps showed two regions of well-defined helical density near the CDRs of each Fab. These regions were modeled as the peptide, and the structure refined to an R_{crys}/R_{free}=21.3/27.4%; see Table 3.

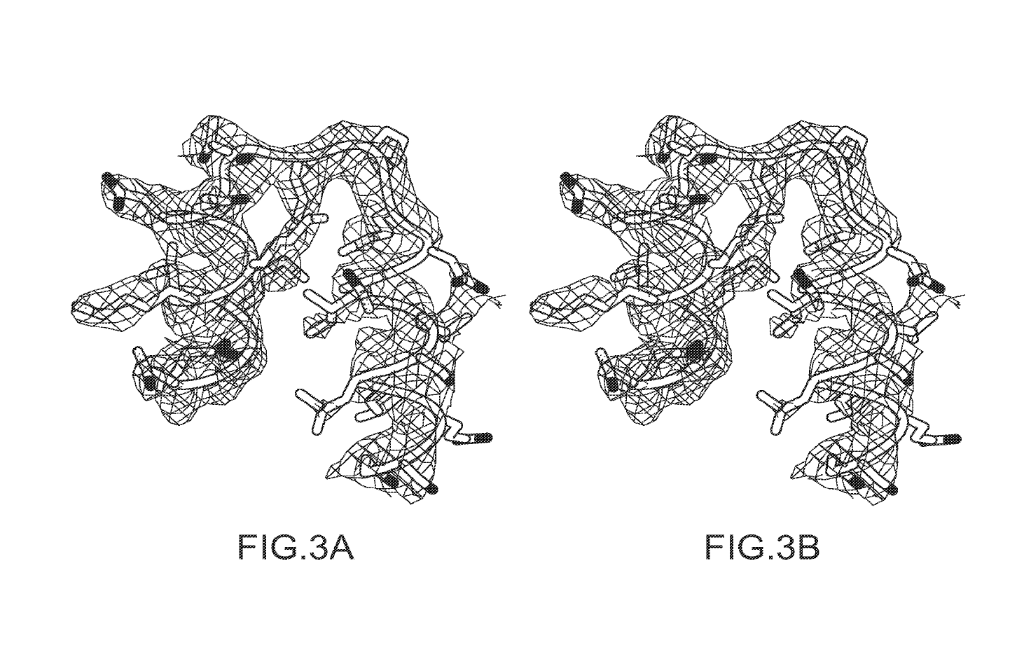

The peptide forms a helix-loop-helix (FIG. 1b), in agreement with secondary structure predictions of the RSV F glycoprotein (Smith B J et al., 2002, Protein Eng. 15, 365-371). The main-chain electron density for the peptide was good for all residues and the side chain density was good for residues 262-276, but weak or non-existent for residues N- and C-terminal to this region (FIG. 3). The variable domains of the peptide-bound motavizumab structure and the unbound palivizumab structure are similar (rmsd 1.8 Å for variable domain Ca), with the largest differences occurring in the three heavy chain CDRs.

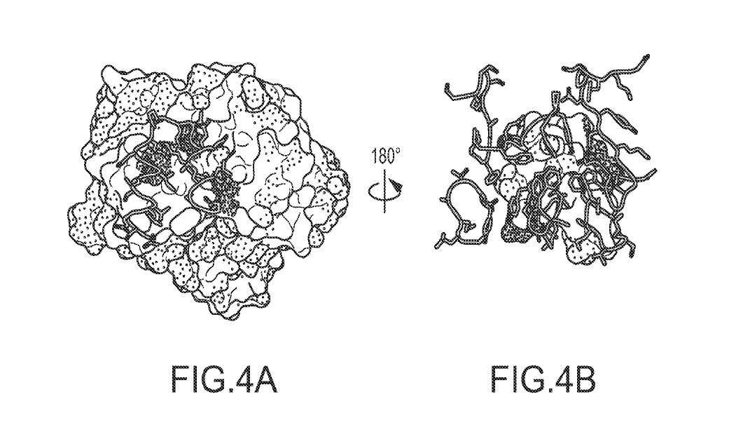

To understand the structural basis for the high affinity interaction between motavizumab and the RSV F protein, the structure of the peptide/Fab complex was analyzed. The interface between the peptide and Fab buries a total of 1,304 Å² of surface area (680 Å² on the peptide and 624 Å² on the Fab, as calculated by PISA, Krissinel E et al., 2007, J. Mol. Biol. 372, 774-797) and has a shape complementarity (S_{c}) value of 0.76, which is substantially higher than the typical range of 0.64-0.68 for antibody/antigen complexes (Lawrence M C et al., 1993, J. Mol. Biol. 234, 946-950). The electrostatic potentials on the surface of the peptide and Fab are also complementary, with several acidic patches on the Fab interacting with positively charged regions on the peptide (FIG. 4). Approximately 73% of the surface area buried on the Fab is located on the heavy chain, which possesses a large hydrophobic region consisting of residues from the second and third CDRs (FIG. 1c). This region contacts peptide residues located along the length of both helices. The four peptide residues between the two helices do not contribute significantly to motavizumab binding to RSV F, having only 8 Å² buried at the interface. Interactions between the peptide and heavy chain include hydrogen bonds formed between the peptide side chain of Asn262 and the Fab side chains of Asp54 and Lys56, as well as a hydrogen bond between the peptide side chain of Ser275 and the carbonyl oxygen of Fab residue Ile97. There are also several interactions between the peptide and light chain. These include a hydrogen bond between the side chain of peptide residue Asn268 and the carbonyl oxygen of Gly90, as well as a salt-bridge between the peptide side chain of Lys272 and the side chain of Asp49 in the second CDR (FIG. 1c).

The interactions between the peptide and motavizumab Fab are consistent with RSV F glycoprotein mutations known to disrupt antibody binding to this epitope. It has been demonstrated that mutations N262Y, N268I and K272E decrease the binding of several antibodies that recognize this region of the F glycoprotein (Arbiza J et al., ibid.). The mutations K272M and K272Q have also been found in RSV F glycoprotein escape mutants that are resistant to palivizumab (Zhao X et al., 2004, J. Infect. Dis. 190, 1941-1946). The side chains of these three peptide residues all form hydrogen bonds or salt bridges with residues in the Fab (FIG. 1c), interactions that would be lost by the mutations listed above.

To investigate the structural basis for motavizumab's enhanced potency over palivizumab, the positions of the 7 altered residues that increase the affinity to the F glycoprotein were analyzed in the peptide-bound crystal structure (FIG. 1d). Three of the seven altered residues (S32A, T98F and W100F in the heavy chain) directly contact the peptide and are located in the large hydrophobic patch described earlier. Both the S32A and T98F substitutions increase the hydrophobicity of this patch, favoring interactions with the peptide. As for the W100F mutation, the smaller Phe side chain is able to pack tightly against peptide residues N268 and K272. The larger Trp side chain found in palivizumab would likely alter the conformation of these residues, which make hydrogen bond and salt bridge interactions with the Fab, respectively. When the three palivizumab residues were modeled into the complex, the S_{c} value decreased from 0.76 to 0.70, reflecting the poorer fit between the peptide and Fab.

The other four substitutions that increase the potency of motavizumab do not contact the peptide directly. Two of the mutations (D58H and S95D in the heavy chain) are located near the interface with the peptide, and their side chains interact with other residues in the CDRs. Thus, they likely exert their effects indirectly by altering the position of other amino acids that do contact the peptide. The side chains of the two remaining substitutions, S65D in the heavy chain and S29R in the light chain, have weak electron density and do not contact any residues in the peptide or Fab. However, both substitutions increase the on-rate of motavizumab for the F glycoprotein, and the S29R mutation alone results in a 4.4-fold increase in RSV neutralization in vitro⁹. Collectively, these data suggest that the S65D and S29R side chains either bind to residues in the F glycoprotein located outside the primary epitope or increase favorable long-range electrostatic interactions. Relevant to this, motavizumab binds to the peptide 6.000-fold weaker than the full-length F protein (230 nM vs 0.035 nM) (Wu H et al., 2007, ibid.; Tous G I et al., 2006, U.S. patent application Ser. No. 11/230,593), though some fraction of the decrease in peptide affinity is likely due to the peptide not adopting the helix-loop-helix conformation in solution (Lopez J A et al., 1993, J. Gen. Virol. 74, 2567-2577).

An earlier version of motavizumab contained residues Phe52, Phe53, and Asp55 in the light chain CDR2, which increased in vitro RSV neutralization ˜2-fold (Wu H et al., 2007, ibid.). However, these residues also increased non-specific tissue binding and decreased the in vivo potency (Wu H et al., 2007, ibid.), perhaps due to the two solvent-exposed Phe residues (FIG. 1d). Thus, they were ultimately returned to the amino acids found in palivizumab (Ser52, Lys53, and Ala55).

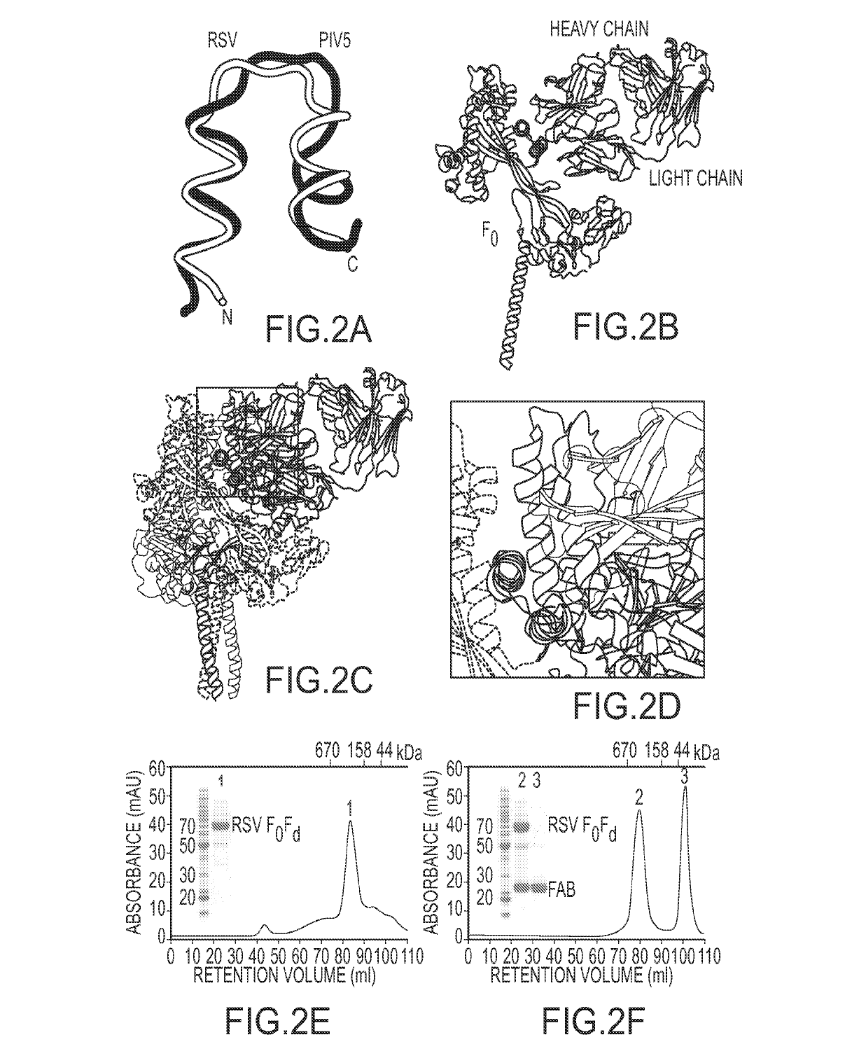

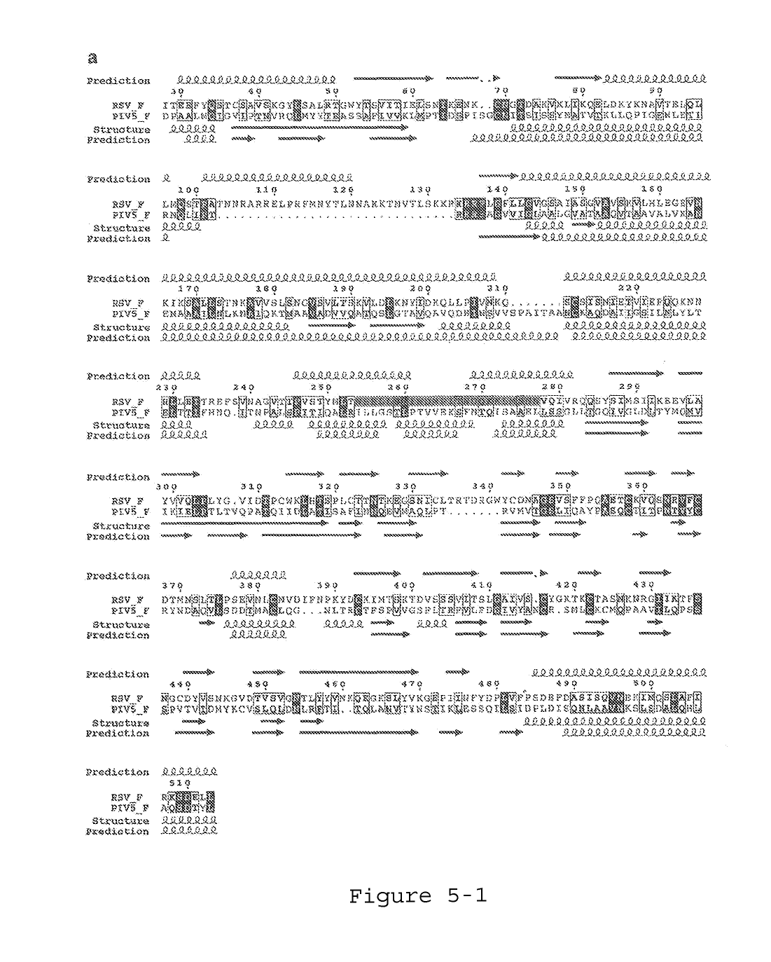

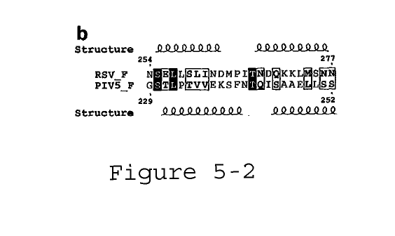

To visualize the binding of motavizumab to the full-length F glycoprotein, a model was generated based on the pre-fusion parainfluenza virus 5 (PIV5) structure (Yin H S et al., 2006, Nature 439, 38-44) (12.4% sequence identity to RSV F (Smith B J et al., ibid.)). A sequence alignment (FIG. 5a) identified a similar helix-loop-helix, and structural analysis provided a precise alignment (FIG. 5b), shifting the PIV5 sequence by three amino acids to provide a motavizumab epitope/PIV5 superposition of 2.1 Å rmsd for all 24 peptide Ca atoms (FIG. 2a). A model was generated by orienting the Fab via superposition of the bound peptide onto the corresponding epitope in the PIV5 F glycoprotein structure. The resulting model shows no clashes between the Fab and F glycoprotein monomer to which it is bound (FIG. 2b).

In the pre-fusion trimeric context, however, both the heavy and light chains of the Fab clash with an adjacent RSV F monomer that packs against the same face of the helix-loop-helix that motavizumab binds (FIG. 2c,d). The location of the epitope at a subunit interface may explain why this neutralizing epitope is so highly conserved in RSV strains. Since motavizumab neutralizes RSV by preventing the fusion of the viral and cellular membranes (Johnson S et al., 1997, ibid.), motavizumab must bind to the F glycoprotein before or during the transition to the post-fusion state. The extensive clashes in the trimer model, however, suggest that motavizumab would be unable to bind the pre-fusion trimeric F glycoprotein as it exists in the PIV5 structure. To address this issue experimentally, a soluble RSV F glycoprotein was expressed and purified in a form similar to the PIV5 F glycoprotein used in the modeling. Specifically, the known furin cleavage sites were mutated and a fibritin trimerization motif (Tao Y et al., 1997, Structure 5, 789-798) was appended to the truncated C terminus to keep the protein in a trimeric, pre-fusion conformation. This stabilized RSV F glycoprotein, referred to as RSV F₀ Fd, eluted from a gel filtration column with a retention volume consistent with that of a glycosylated trimer (FIG. 2e). To determine whether motavizumab or palivizumab is able to bind the pre-fusion trimeric RSV F glycoprotein, palivizumab Fab was added in excess to a solution of RSV F₀ Fd, and the mixture was passed over a gel filtration column. The elution profile contained two peaks, corresponding to excess Fab and a complex of the Fab and F glycoprotein (FIG. 2f). The elution volume of the complex peak was consistent with a trimeric F glycoprotein bound by three Fabs, in agreement with the ratio (1:2.97) of F glycoprotein and Fab bands observed on a Coomassie stained SDS-PAGE gel containing fractions from the complex peak (FIG. 2f).

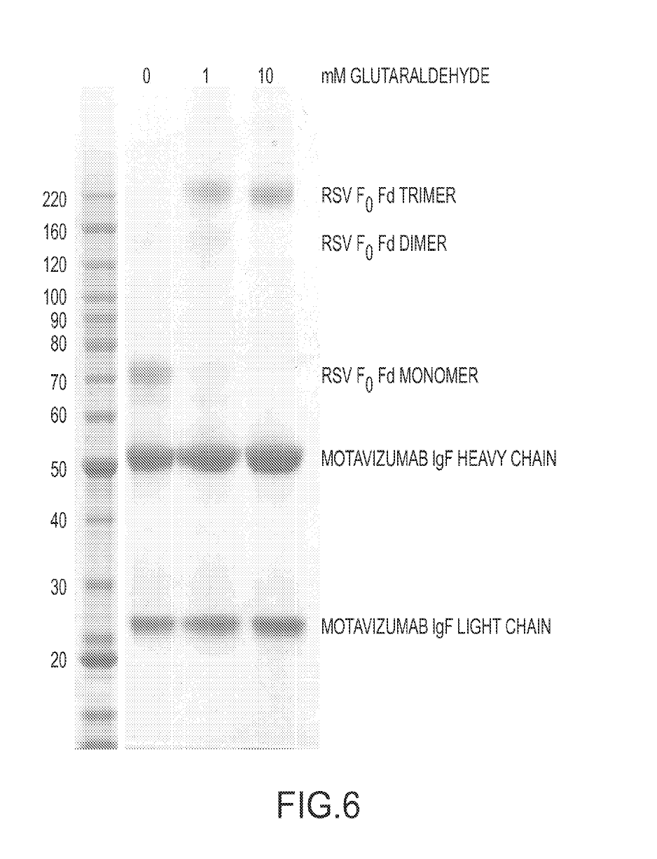

Collectively, these data suggest that motavizumab binds to or induces a conformation of the trimeric F glycoprotein that is different from that observed in the PIV5 F pre-fusion structure. One possibility is that the structure of the RSV F glycoprotein differs significantly from that of PIV5, although the predicted RSV F glycoprotein secondary structure appears similar to that observed in the PIV5 F pre-fusion crystal structure (FIG. 5). Another possibility is that motavizumab traps an intermediate between pre- and post-fusion forms. It has been suggested that during this transition, which is one of the largest structural rearrangements known, the F glycoprotein monomers transiently dissociate prior to forming the trimeric post-fusion conformation (Yin H S et al., ibid). It is to be noted in this regard that glutaraldehyde crosslinking of the soluble F glycoprotein trimer does not inhibit motavizumab binding (FIG. 6). Alternatively, the RSV F glycoprotein in its pre-fusion conformation may contain sufficient flexibility to bind three motavizumab Fabs. Modeling studies indicate that a ˜30° rotation of domain III parallel to the 3-fold axis would allow clash-free binding of three Fabs. A similar degree of rotation has been observed in cryo-EM tomograms after the binding of neutralizing antibodies to Dengue virus (Lok S-M, et al., 2008, Nat Struct Mol Biol 15, 312-317) and HIV-1 (Liu J et al., 2008, Nature 455, 109-113). Such flexibility may be a more common feature of viral fusion proteins than previously thought.

### Materials and Methods

a. Cloning, expression and purification of motavizumab IgG. Two DNA fragments encoding the variable heavy and light chains of motavizumab (Wu H et al., 2007, J. Mol. Biol. 368, 652-665) with appropriate signal sequences were synthesized by GeneArt (Regensburg, Germany) and cloned in-frame into mammalian expression vectors containing human IgG1 heavy and light constant domains, respectively. The amino acid sequence of the variable heavy chain of motavizumab is as follows: QVTLRESGPA LVKPTQTLTL TCTFSGFSLS TAGMSVGWIR QPPGKALEWL ADIWWDDKKH YNPSLKDRLT ISKDTSKNQV VLKVTNMDPA DTATYYCARD MIFNFYFDVW GQGTTVTVSS, also denoted herein as SEQ ID NO:5. The amino acid sequence of the variable light chain of motavizumab is as follows: DIQMTQSPST LSASVGDRVT ITCSASSRVG YMHWYQQKPG KAPKLLIYDT SKLASGVPSR FSGSGSGTEF TLTISSLQPD DFATYYCFQG SGYPFTFGGG TKVEIK, also denoted herein as SEQ ID NO:6.

Both vectors were co-transfected at a 1:1 ratio into HEK293F cells (Invitrogen, Life Technologies, Carlsbad, Calif.) in serum-free 293Freestyle medium (Invitrogen). After 3 hours, valproic acid (Sigma, St. Louis, Mo.) was added to 4 mM final concentration. Expression lasted for four days at 37° C. with 10% CO2 and shaking at 125 rpm in disposable flasks. The supernatant was collected, filtered, and passed over 5 ml of Protein A agarose resin (Pierce). After washing with several column volumes of phosphate-buffered saline (PBS), the resin was eluted with 25 ml of IgG Elution Buffer (Pierce, Thermo Fisher Scientific, Rockford, Ill.) and immediately neutralized with 1 M Tris pH 8.0. The eluted protein was dialyzed against PBS and stored at 4° C.

b. Measurement of antibody-mediated neutralization. RSV expressing green fluorescent protein (GFP) was provided by Mark Peeples and Peter Collins and constructed as previously reported (Hallak L K, et al., 2000, Virology 271, 264-275). Antibody-mediated neutralization was measured using HEp-2 cells. GFP-RSV was added to serial four-fold dilutions of serum and/or antibody in 96-well plates and incubated at 37° C. for one hour. Serum concentrations ranged from 1:10 to 1:40,960. After one hour, 100 μl of virus/serum mixture was added to 5×10⁴ cells/100 μl per well in 96-well plates. Infection was monitored as a function of GFP expression (encoded by the viral genome) at 18 hours post-infection by flow cytometry (LSR II, BD Bioscience, CA, USA). Prior to assessment by flow cytometry, cells were treated with trypsin to ensure a single-cell suspension optimal for analysis and fixed with 0.5% paraformaldehyde, Data were analyzed by curve fitting and non-linear regression (GraphPad Prism, GraphPad Software Inc., San Diego Calif.) in order to demonstrate the percent neutralization at a given antibody concentration, and the neutralization activity was compared based on the EC50.

c. Digestion and purification of motavizumab Fab fragments. The purified motavizumab IgG protein was reduced with 100 mM dithiothreitol at 37° C. for 1 hour and then alkylated with 2 mM iodoacetamide for 48 hours at 4° C. 10 ml of reduced and alkylated IgG in PBS at 3.5 mg/ml was combined with 15 μg of endoproteinase Lys-C (Roche) and incubated at 37° C. for 6 hours. The reaction was quenched by the addition of TLCK and leupeptin to 50 μg/ml and 2 μg/ml, respectively. To remove the Fc fragments from the Fab fragments, the quenched reaction was passed over 5 ml of Protein A agarose. The Fab-containing flow through was further purified over an S200 gel filtration column and concentrated aliquots were stored frozen at −80° C.

d. Protein crystallization and data collection. A peptide with the sequence NSELLSLIND MPITNDQKKL MSNN, corresponding to residues 254-277 of the RSV F protein and also denoted herein as SEQ ID NO:2) was synthesized by American Peptide (Sunnyvale, Calif.) with an acetylated N-terminus and an amidated C-terminus. A five-fold molar excess of peptide was incubated with motavizumab Fab at 22° C. for 1.5 hours and then concentrated to give a 13.1 mg/ml solution of Fab/peptide complex in 2 mM Tris pH 7.5, 150 mM NaCl. Initial crystals were grown by the vapor diffusion method in sitting drops at 20° C. by mixing 0.1 μl of protein complex with 0.1 μl of reservoir solution (17.5% (w/v) PEG 8000, 0.2 M zinc acetate, 0.1 M cacodylate pH 6.5) using a Cartesian Honeybee crystallization robot (Genomic Solutions). These initial crystals were streak-seeded into hanging drops consisting of 1 μl protein complex and 1 μl 30% (w/v) PEG 1500. After several days rectangular crystals appeared in a single drop with dimensions 40×40×10 μm. These crystals were flash frozen in liquid nitrogen in 40% (w/v) PEG 1500, 30% (v/v) ethylene glycol and loaded into a cryopuck. Data were collected at a wavelength of 0.82656 Å at the SER-CAT beamline ID-22 using the robot automounter (Advanced Photon Source, Argonne National Laboratory).

e. Structure determination, model building and refinement. Diffraction data were processed with the HKL2000 suite (Otwinowski Z et al., 1997, Methods Enzymol. 276, 307-326, Academic Press) and a molecular replacement solution was found by PHASER (McCoy A J et al, 2007, J. Appl. Crystallog. 40, 658-674) using the palivizumab Fab structure (PDB ID: 2hwz) as a search model. Two Fab molecules were placed in the asymmetric unit. After rigid body and TLS refinement using PHENIX (Adams, P D et al., 2002, Acta Crystallogr., Section D 58, 1948-1954), helical peptide density was evident near the CDRs of each Fab. Model building was carried out using COOT (Emsley, P et al., 2004, Acta Crystallogr., Section D 60, 2126-2132) and refinement was performed with PHENIX using NCS restraints. Final data collection and refinement statistics are presented in Table 3. The atomic coordinates for the motavizumab/peptide complex have been deposited in the Protein Data Bank under PDB accession code 3IXT. The atomic coordinates for the peptide portion of the complex are indicated below in Table 6.

TABLE 6
Atomic coordinates for the motavizumab binding peptide

| | | | | | | | | | | | | |
|---|---|---|---|---|---|---|---|---|---|---|---|---|
| ATOM | 3243 | N | ASN | P | 254 | 7.154 | 8.372 | −9.660 | 1.00 | 124.11 | | N |
| ANISOU | 3243 | N | ASN | P | 254 | 13767 | 16195 | 17192 | −3482 | 2530 | 2430 | N |
| ATOM | 3244 | CA | ASN | P | 254 | 7.612 | 9.736 | −9.412 | 1.00 | 121.53 | | C |
| ANISOU | 3244 | CA | ASN | P | 254 | 13313 | 15765 | 17097 | −3858 | 2355 | 2424 | C |
| ATOM | 3245 | C | ASN | P | 254 | 6.768 | 10.583 | −8.462 | 1.00 | 111.67 | | C |
| ANISOU | 3245 | C | ASN | P | 254 | 12385 | 14386 | 15658 | −4047 | 1969 | 2168 | C |
| ATOM | 3246 | O | ASN | P | 254 | 5.706 | 11.098 | −8.827 | 1.00 | 107.07 | | O |
| ANISOU | 3246 | O | ASN | P | 254 | 12235 | 13626 | 14819 | −4027 | 2035 | 2025 | O |
| ATOM | 3247 | CB | ASN | P | 254 | 7.853 | 10.480 | −10.730 | 1.00 | 123.56 | | C |
| ANISOU | 3247 | CB | ASN | P | 254 | 13592 | 15864 | 17490 | −3953 | 2745 | 2570 | C |
| ATOM | 3248 | CG | ASN | P | 254 | 9.265 | 10.333 | −11.225 | 1.00 | 130.78 | | C |
| ANISOU | 3248 | CG | ASN | P | 254 | 13976 | 16914 | 18799 | −4017 | 2965 | 2837 | C |
| ATOM | 3249 | OD1 | ASN | P | 254 | 10.213 | 10.485 | −10.461 | 1.00 | 132.18 | | O |
| ANISOU | 3249 | OD1 | ASN | P | 254 | 13727 | 17233 | 19260 | −4212 | 2717 | 2883 | O |
| ATOM | 3250 | ND2 | ASN | P | 254 | 9.416 | 10.009 | −12.511 | 1.00 | 136.19 | | N |
| ANISOU | 3250 | ND2 | ASN | P | 254 | 14673 | 17575 | 19498 | −3837 | 3434 | 3010 | N |
| ATOM | 3251 | N | SER | P | 255 | 7.270 | 10.702 | −7.237 | 1.00 | 104.70 | | N |
| ANISOU | 3251 | N | SER | P | 255 | 11278 | 13608 | 14896 | −4206 | 1562 | 2109 | N |
| ATOM | 3252 | CA | SER | P | 255 | 6.624 | 11.477 | −6.192 | 1.00 | 96.74 | | C |
| ANISOU | 3252 | CA | SER | P | 255 | 10531 | 12499 | 13728 | −4386 | 1160 | 1872 | C |
| ATOM | 3253 | C | SER | P | 255 | 6.465 | 12.942 | −6.584 | 1.00 | 85.08 | | C |
| ANISOU | 3253 | C | SER | P | 255 | 9239 | 10769 | 12318 | −4677 | 1173 | 1822 | C |
| ATOM | 3254 | O | SER | P | 255 | 5.469 | 13.589 | −6.245 | 1.00 | 75.10 | | O |
| ANISOU | 3254 | O | SER | P | 255 | 8390 | 9349 | 10797 | −4716 | 1012 | 1614 | O |
| ATOM | 3255 | CB | SER | P | 255 | 7.430 | 11.374 | −4.900 | 1.00 | 100.06 | | C |
| ANISOU | 3255 | CB | SER | P | 255 | 10618 | 13086 | 14314 | −4514 | 733 | 1850 | C |
| ATOM | 3256 | OG | SER | P | 255 | 8.819 | 11.450 | −5.170 | 1.00 | 101.92 | | O |
| ANISOU | 3256 | OG | SER | P | 255 | 10318 | 13442 | 14965 | −4658 | 796 | 2057 | O |
| ATOM | 3257 | N | GLU | P | 256 | 7.456 | 13.465 | −7.294 | 1.00 | 90.87 | | N |
| ANISOU | 3257 | N | GLU | P | 256 | 9673 | 11460 | 13395 | −4878 | 1375 | 2016 | N |
| ATOM | 3258 | CA | GLU | P | 256 | 7.446 | 14.869 | −7.683 | 1.00 | 100.04 | | C |
| ANISOU | 3258 | CA | GLU | P | 256 | 11006 | 12355 | 14651 | −5184 | 1410 | 1998 | C |
| ATOM | 3259 | C | GLU | P | 256 | 6.561 | 15.110 | −8.898 | 1.00 | 95.96 | | C |
| ANISOU | 3259 | C | GLU | P | 256 | 10919 | 11641 | 13901 | −5014 | 1792 | 2007 | C |
| ATOM | 3260 | O | GLU | P | 256 | 5.860 | 16.117 | −8.970 | 1.00 | 93.69 | | O |
| ANISOU | 3260 | O | GLU | P | 256 | 11032 | 11111 | 13454 | −5117 | 1734 | 1875 | O |
| ATOM | 3261 | CB | GLU | P | 256 | 8.867 | 15.360 | −7.952 | 1.00 | 117.93 | | C |
| ANISOU | 3261 | CB | GLU | P | 256 | 12779 | 14646 | 17381 | −5502 | 1494 | 2201 | C |
| ATOM | 3262 | CG | GLU | P | 256 | 9.623 | 14.514 | −8.951 | 1.00 | 137.85 | | C |
| ANISOU | 3262 | CG | GLU | P | 256 | 14946 | 17336 | 20096 | −5326 | 1918 | 2463 | C |
| ATOM | 3263 | CD | GLU | P | 256 | 10.877 | 15.200 | −9.446 | 1.00 | 155.42 | | C |
| ANISOU | 3263 | CD | GLU | P | 256 | 16748 | 19544 | 22759 | −5665 | 2092 | 2662 | C |
| ATOM | 3264 | OE1 | GLU | P | 256 | 11.414 | 16.058 | −8.713 | 1.00 | 159.74 | | O |
| ANISOU | 3264 | OE1 | GLU | P | 256 | 17121 | 20045 | 23527 | −6047 | 1784 | 2594 | O |
| ATOM | 3265 | OE2 | GLU | P | 256 | 11.323 | 14.883 | −10.568 | 1.00 | 161.28 | | O |
| ANISOU | 3265 | OE2 | GLU | P | 256 | 17337 | 20320 | 23624 | −5559 | 2543 | 2879 | O |
| ATOM | 3266 | N | LEU | P | 257 | 6.597 | 14.186 | −9.853 | 1.00 | 101.08 | | N |
| ANISOU | 3266 | N | LEU | P | 257 | 11497 | 12391 | 14515 | −4733 | 2173 | 2157 | N |
| ATOM | 3267 | CA | LEU | P | 257 | 5.751 | 14.295 | −11.035 | 1.00 | 105.52 | | C |
| ANISOU | 3267 | CA | LEU | P | 257 | 12464 | 12797 | 14833 | −4526 | 2529 | 2159 | C |
| ATOM | 3268 | C | LEU | P | 257 | 4.279 | 14.282 | −10.639 | 1.00 | 92.20 | | C |
| ANISOU | 3268 | C | LEU | P | 257 | 11263 | 11055 | 12715 | −4343 | 2347 | 1883 | C |
| ATOM | 3269 | O | LEU | P | 257 | 3.455 | 14.971 | −11.242 | 1.00 | 80.48 | | O |
| ANISOU | 3269 | O | LEU | P | 257 | 10189 | 9377 | 11014 | −4288 | 2459 | 1793 | O |
| ATOM | 3270 | CB | LEU | P | 257 | 6.036 | 13.160 | −12.018 | 1.00 | 115.65 | | C |
| ANISOU | 3270 | CB | LEU | P | 257 | 13585 | 14225 | 16131 | −4230 | 2932 | 2345 | C |
| ATOM | 3271 | CG | LEU | P | 257 | 7.431 | 13.112 | −12.643 | 1.00 | 125.63 | | C |
| ANISOU | 3271 | CG | LEU | P | 257 | 14378 | 15562 | 17795 | −4348 | 3209 | 2635 | C |
| ATOM | 3272 | CD1 | LEU | P | 257 | 7.455 | 12.109 | −13.788 | 1.00 | 125.83 | | C |
| ANISOU | 3272 | CD1 | LEU | P | 257 | 14393 | 15674 | 17744 | −4003 | 3651 | 2792 | C |
| ATOM | 3273 | CD2 | LEU | P | 257 | 7.861 | 14.487 | −13.125 | 1.00 | 131.86 | | C |
| ANISOU | 3273 | CD2 | LEU | P | 257 | 15206 | 16112 | 18783 | −4685 | 3323 | 2722 | C |
| ATOM | 3274 | N | LEU | P | 258 | 3.961 | 13.490 | −9.619 | 1.00 | 87.87 | | N |
| ANISOU | 3274 | N | LEU | P | 258 | 10664 | 10685 | 12036 | −4239 | 2072 | 1749 | N |
| ATOM | 3275 | CA | LEU | P | 258 | 2.600 | 13.395 | −9.107 | 1.00 | 83.75 | | C |
| ANISOU | 3275 | CA | LEU | P | 258 | 10548 | 10153 | 11119 | −4083 | 1890 | 1479 | C |
| ATOM | 3276 | C | LEU | P | 258 | 2.187 | 14.657 | −8.352 | 1.00 | 89.10 | | C |
| ANISOU | 3276 | C | LEU | P | 258 | 11470 | 10660 | 11724 | −4309 | 1559 | 1292 | C |
| ATOM | 3277 | O | LEU | P | 258 | 1.104 | 15.199 | −8.579 | 1.00 | 87.97 | | O |
| ANISOU | 3277 | O | LEU | P | 258 | 11742 | 10389 | 11292 | −4214 | 1564 | 1118 | O |
| ATOM | 3278 | CB | LEU | P | 258 | 2.460 | 12.175 | −8.196 | 1.00 | 74.89 | | C |
| ANISOU | 3278 | CB | LEU | P | 258 | 9305 | 9260 | 9891 | −3929 | 1714 | 1410 | C |
| ATOM | 3279 | CG | LEU | P | 258 | 2.444 | 10.811 | −8.889 | 1.00 | 75.87 | | C |
| ANISOU | 3279 | CG | LEU | P | 258 | 9350 | 9528 | 9951 | −3633 | 2030 | 1520 | C |
| ATOM | 3280 | CD1 | LEU | P | 258 | 2.583 | 9.674 | −7.882 | 1.00 | 52.50 | | C |
| ANISOU | 3280 | CD1 | LEU | P | 258 | 6237 | 6763 | 6946 | −3528 | 1837 | 1495 | C |
| ATOM | 3281 | CD2 | LEU | P | 258 | 1.177 | 10.648 | −9.714 | 1.00 | 71.13 | | C |
| ANISOU | 3281 | CD2 | LEU | P | 258 | 9153 | 8864 | 9008 | −3407 | 2256 | 1381 | C |
| ATOM | 3282 | N | SER | P | 259 | 3.050 | 15.118 | −7.452 | 1.00 | 84.33 | | N |
| ANISOU | 3282 | N | SER | P | 259 | 10608 | 10060 | 11373 | −4593 | 1263 | 1317 | N |
| ATOM | 3283 | CA | SER | P | 259 | 2.753 | 16.304 | −6.662 | 1.00 | 78.21 | | C |
| ANISOU | 3283 | CA | SER | P | 259 | 10063 | 9112 | 10542 | −4822 | 925 | 1137 | C |
| ATOM | 3284 | C | SER | P | 259 | 2.572 | 17.520 | −7.562 | 1.00 | 81.91 | | C |
| ANISOU | 3284 | C | SER | P | 259 | 10820 | 9288 | 11015 | −4936 | 1112 | 1161 | C |
| ATOM | 3285 | O | SER | P | 259 | 1.932 | 18.496 | −7.175 | 1.00 | 92.12 | | O |
| ANISOU | 3285 | O | SER | P | 259 | 12470 | 10393 | 12137 | −5016 | 910 | 979 | O |
| ATOM | 3286 | CB | SER | P | 259 | 3.845 | 16.550 | −5.623 | 1.00 | 86.96 | | C |
| ANISOU | 3286 | CB | SER | P | 259 | 10806 | 10283 | 11954 | −5123 | 583 | 1172 | C |
| ATOM | 3287 | OG | SER | P | 259 | 5.128 | 16.495 | −6.216 | 1.00 | 111.78 | | O |
| ANISOU | 3287 | OG | SER | P | 259 | 13511 | 13467 | 15494 | −5284 | 782 | 1427 | O |
| ATOM | 3288 | N | LEU | P | 260 | 3.135 | 17.452 | −8.765 | 1.00 | 80.16 | | N |
| ANISOU | 3288 | N | LEU | P | 260 | 10467 | 9019 | 10971 | −4923 | 1507 | 1389 | N |
| ATOM | 3289 | CA | LEU | P | 260 | 2.966 | 18.509 | −9.754 | 1.00 | 81.64 | | C |
| ANISOU | 3289 | CA | LEU | P | 260 | 10963 | 8919 | 11138 | −4989 | 1747 | 1442 | C |
| ATOM | 3290 | C | LEU | P | 260 | 1.590 | 18.417 | −10.392 | 1.00 | 82.16 | | C |
| ANISOU | 3290 | C | LEU | P | 260 | 11501 | 8937 | 10780 | −4636 | 1897 | 1295 | C |
| ATOM | 3291 | O | LEU | P | 260 | 0.872 | 19.411 | −10.499 | 1.00 | 82.48 | | O |
| ANISOU | 3291 | O | LEU | P | 260 | 11967 | 8754 | 10617 | −4633 | 1836 | 1160 | O |
| ATOM | 3292 | CB | LEU | P | 260 | 4.035 | 18.407 | −10.840 | 1.00 | 88.21 | | C |
| ANISOU | 3292 | CB | LEU | P | 260 | 11497 | 9732 | 12286 | −5076 | 2148 | 1745 | C |
| ATOM | 3293 | CG | LEU | P | 260 | 5.418 | 18.965 | −10.516 | 1.00 | 96.65 | | C |
| ANISOU | 3293 | CG | LEU | P | 260 | 12153 | 10763 | 13805 | −5501 | 2072 | 1901 | C |
| ATOM | 3294 | CD1 | LEU | P | 260 | 6.324 | 18.821 | −11.725 | 1.00 | 101.88 | | C |
| ANISOU | 3294 | CD1 | LEU | P | 260 | 12556 | 11421 | 14733 | −5537 | 2540 | 2197 | C |
| ATOM | 3295 | CD2 | LEU | P | 260 | 5.316 | 20.420 | −10.084 | 1.00 | 95.37 | | C |
| ANISOU | 3295 | CD2 | LEU | P | 260 | 12284 | 10293 | 13658 | −5824 | 1844 | 1782 | C |
| ATOM | 3296 | N | ILE | P | 261 | 1.234 | 17.212 | −10.823 | 1.00 | 88.03 | | N |
| ANISOU | 3296 | N | ILE | P | 261 | 12167 | 9892 | 11387 | −4332 | 2090 | 1316 | N |
| ATOM | 3297 | CA | ILE | P | 261 | −0.060 | 16.970 | −11.445 | 1.00 | 90.12 | | C |
| ANISOU | 3297 | CA | ILE | P | 261 | 12819 | 10164 | 11258 | −3990 | 2230 | 1161 | C |
| ATOM | 3298 | C | ILE | P | 261 | −1.191 | 17.335 | −10.489 | 1.00 | 88.77 | | C |
| ANISOU | 3298 | C | ILE | P | 261 | 12956 | 9997 | 10776 | −3935 | 1882 | 852 | C |
| ATOM | 3299 | O | ILE | P | 261 | −2.217 | 17.877 | −10.901 | 1.00 | 91.98 | | O |
| ANISOU | 3299 | O | ILE | P | 261 | 13765 | 10301 | 10884 | −3760 | 1914 | 694 | O |
| ATOM | 3300 | CB | ILE | P | 261 | −0.206 | 15.502 | −11.893 | 1.00 | 84.69 | | C |
| ANISOU | 3300 | CB | ILE | P | 261 | 11965 | 9720 | 10492 | −3712 | 2451 | 1212 | C |
| ATOM | 3301 | CG1 | ILE | P | 261 | 0.855 | 15.159 | −12.941 | 1.00 | 83.55 | | C |
| ANISOU | 3301 | CG1 | ILE | P | 261 | 11551 | 9571 | 10622 | −3717 | 2830 | 1515 | C |
| ATOM | 3302 | CG2 | ILE | P | 261 | −1.598 | 15.246 | −12.447 | 1.00 | 77.49 | | C |
| ANISOU | 3302 | CG2 | ILE | P | 261 | 11434 | 8840 | 9169 | −3386 | 2552 | 1011 | C |
| ATOM | 3303 | CD1 | ILE | P | 261 | 0.798 | 13.729 | −13.426 | 1.00 | 52.78 | | C |
| ANISOU | 3303 | CD1 | ILE | P | 261 | 7511 | 5886 | 6658 | −3439 | 3060 | 1577 | C |
| ATOM | 3304 | N | ASN | P | 262 | −0.990 | 17.044 | −9.209 | 1.00 | 83.99 | | N |
| ANISOU | 3304 | N | ASN | P | 262 | 12161 | 9519 | 10233 | −4069 | 1551 | 766 | N |
| ATOM | 3305 | CA | ASN | P | 262 | −1.986 | 17.356 | −8.193 | 1.00 | 82.83 | | C |
| ANISOU | 3305 | CA | ASN | P | 262 | 12280 | 9392 | 9801 | −4027 | 1218 | 479 | C |
| ATOM | 3306 | C | ASN | P | 262 | −2.127 | 18.861 | −7.981 | 1.00 | 87.67 | | C |
| ANISOU | 3306 | C | ASN | P | 262 | 13199 | 9728 | 10385 | −4198 | 1038 | 385 | C |
| ATOM | 3307 | O | ASN | P | 262 | −3.234 | 19.372 | −7.812 | 1.00 | 83.35 | | O |
| ANISOU | 3307 | O | ASN | P | 262 | 13029 | 9126 | 9514 | −4047 | 926 | 157 | O |
| ATOM | 3308 | CB | ASN | P | 262 | −1.644 | 16.661 | −6.874 | 1.00 | 78.11 | | C |
| ANISOU | 3308 | CB | ASN | P | 262 | 11416 | 8988 | 9275 | −4124 | 919 | 432 | C |
| ATOM | 3309 | CG | ASN | P | 262 | −2.826 | 16.596 | −5.930 | 1.00 | 77.84 | | C |
| ANISOU | 3309 | CG | ASN | P | 262 | 11645 | 9043 | 8886 | −3998 | 662 | 140 | C |
| ATOM | 3310 | OD1 | ASN | P | 262 | −3.953 | 16.917 | −6.307 | 1.00 | 76.30 | | O |
| ANISOU | 3310 | OD1 | ASN | P | 262 | 11788 | 8814 | 8388 | −3808 | 726 | −36 | O |
| ATOM | 3311 | ND2 | ASN | P | 262 | −2.576 | 16.177 | −4.698 | 1.00 | 81.46 | | N |
| ANISOU | 3311 | ND2 | ASN | P | 262 | 11948 | 9632 | 9373 | −4089 | 373 | 85 | N |
| ATOM | 3312 | N | ASP | P | 263 | −0.999 | 19.566 | −8.004 | 1.00 | 97.04 | | N |
| ANISOU | 3312 | N | ASP | P | 263 | 14221 | 10740 | 11909 | −4514 | 1020 | 558 | N |
| ATOM | 3313 | CA | ASP | P | 263 | −0.983 | 21.013 | −7.804 | 1.00 | 100.49 | | C |
| ANISOU | 3313 | CA | ASP | P | 263 | 14953 | 10871 | 12357 | −4727 | 858 | 490 | C |
| ATOM | 3314 | C | ASP | P | 263 | −1.686 | 21.770 | −8.934 | 1.00 | 102.79 | | C |
| ANISOU | 3314 | C | ASP | P | 263 | 15690 | 10934 | 12433 | −4545 | 1113 | 477 | C |
| ATOM | 3315 | O | ASP | P | 263 | −2.186 | 22.876 | −8.727 | 1.00 | 106.47 | | O |
| ANISOU | 3315 | O | ASP | P | 263 | 16552 | 11164 | 12737 | −4576 | 960 | 335 | O |
| ATOM | 3316 | CB | ASP | P | 263 | 0.455 | 21.520 | −7.650 | 1.00 | 102.30 | | C |
| ANISOU | 3316 | CB | ASP | P | 263 | 14866 | 10975 | 13029 | −5142 | 815 | 689 | C |
| ATOM | 3317 | CG | ASP | P | 263 | 1.092 | 21.093 | −6.337 | 1.00 | 108.70 | | C |
| ANISOU | 3317 | CG | ASP | P | 263 | 15314 | 11971 | 14017 | −5337 | 452 | 646 | C |
| ATOM | 3318 | OD1 | ASP | P | 263 | 0.402 | 20.464 | −5.506 | 1.00 | 114.86 | | O |
| ANISOU | 3318 | OD1 | ASP | P | 263 | 16137 | 12945 | 14560 | −5155 | 235 | 467 | O |
| ATOM | 3319 | OD2 | ASP | P | 263 | 2.291 | 21.390 | −6.141 | 1.00 | 105.41 | | O |
| ANISOU | 3319 | OD2 | ASP | P | 263 | 14566 | 11512 | 13972 | −5673 | 387 | 789 | O |
| ATOM | 3320 | N | MET | P | 264 | −1.719 | 21.172 | −10.122 | 1.00 | 99.31 | | N |
| ANISOU | 3320 | N | MET | P | 264 | 15204 | 10558 | 11973 | −4337 | 1494 | 624 | N |
| ATOM | 3321 | CA | MET | P | 264 | −2.353 | 21.793 | −11.286 | 1.00 | 101.70 | | C |
| ANISOU | 3321 | CA | MET | P | 264 | 15924 | 10663 | 12055 | −4121 | 1756 | 628 | C |
| ATOM | 3322 | C | MET | P | 264 | −3.853 | 21.998 | −11.096 | 1.00 | 101.82 | | C |
| ANISOU | 3322 | C | MET | P | 264 | 16354 | 10719 | 11614 | −3798 | 1606 | 325 | C |
| ATOM | 3323 | O | MET | P | 264 | −4.487 | 21.285 | −10.318 | 1.00 | 107.68 | | O |
| ANISOU | 3323 | O | MET | P | 264 | 17006 | 11714 | 12195 | −3678 | 1411 | 137 | O |
| ATOM | 3324 | CB | MET | P | 264 | −2.113 | 20.952 | −12.540 | 1.00 | 102.27 | | C |
| ANISOU | 3324 | CB | MET | P | 264 | 15845 | 10841 | 12173 | −3929 | 2178 | 829 | C |
| ATOM | 3325 | CG | MET | P | 264 | −0.678 | 20.946 | −13.026 | 1.00 | 108.04 | | C |
| ANISOU | 3325 | CG | MET | P | 264 | 16229 | 11494 | 13326 | −4205 | 2416 | 1147 | C |
| ATOM | 3326 | SD | MET | P | 264 | −0.528 | 20.158 | −14.640 | 1.00 | 100.61 | | S |
| ANISOU | 3326 | SD | MET | P | 264 | 15237 | 10623 | 12367 | −3926 | 2946 | 1371 | S |
| ATOM | 3327 | CE | MET | P | 264 | −1.176 | 18.532 | −14.285 | 1.00 | 169.29 | | C |
| ANISOU | 3327 | CE | MET | P | 264 | 23718 | 19718 | 20885 | −3627 | 2880 | 1227 | C |
| ATOM | 3328 | N | PRO | P | 265 | −4.429 | 22.971 | −11.821 | 1.00 | 98.01 | | N |
| ANISOU | 3328 | N | PRO | P | 265 | 16332 | 9993 | 10916 | −3647 | 1708 | 279 | N |
| ATOM | 3329 | CA | PRO | P | 265 | −5.855 | 23.274 | −11.736 | 1.00 | 96.48 | | C |
| ANISOU | 3329 | CA | PRO | P | 265 | 16538 | 9840 | 10281 | −3308 | 1576 | −11 | C |
| ATOM | 3330 | C | PRO | P | 265 | −6.600 | 22.538 | −12.832 | 1.00 | 99.88 | | C |
| ANISOU | 3330 | C | PRO | P | 265 | 17031 | 10449 | 10470 | −2920 | 1854 | −35 | C |
| ATOM | 3331 | O | PRO | P | 265 | −6.903 | 23.135 | −13.862 | 1.00 | 107.99 | | O |
| ANISOU | 3331 | O | PRO | P | 265 | 18396 | 11298 | 11338 | −2724 | 2054 | 4 | O |
| ATOM | 3332 | CB | PRO | P | 265 | −5.906 | 24.778 | −12.034 | 1.00 | 96.43 | | C |
| ANISOU | 3332 | CB | PRO | P | 265 | 17008 | 9434 | 10197 | −3353 | 1558 | −9 | C |
| ATOM | 3333 | CG | PRO | P | 265 | −4.471 | 25.182 | −12.423 | 1.00 | 101.02 | | C |
| ANISOU | 3333 | CG | PRO | P | 265 | 17421 | 9763 | 11198 | −3739 | 1743 | 308 | C |
| ATOM | 3334 | CD | PRO | P | 265 | −3.756 | 23.909 | −12.728 | 1.00 | 99.21 | | C |
| ANISOU | 3334 | CD | PRO | P | 265 | 16674 | 9802 | 11219 | −3787 | 1950 | 497 | C |
| ATOM | 3335 | N | ILE | P | 266 | −6.871 | 21.257 | −12.630 | 1.00 | 92.24 | | N |
| ANISOU | 3335 | N | ILE | P | 266 | 15761 | 9817 | 9470 | −2812 | 1869 | −98 | N |
| ATOM | 3336 | CA | ILE | P | 266 | −7.583 | 20.481 | −13.634 | 1.00 | 85.03 | | C |
| ANISOU | 3336 | CA | ILE | P | 266 | 14890 | 9088 | 8331 | −2466 | 2115 | −145 | C |
| ATOM | 3337 | C | ILE | P | 266 | −8.590 | 19.538 | −12.993 | 1.00 | 86.91 | | C |
| ANISOU | 3337 | C | ILE | P | 266 | 15019 | 9666 | 8336 | −2297 | 1962 | −412 | C |
| ATOM | 3338 | O | ILE | P | 266 | −8.549 | 19.293 | −11.785 | 1.00 | 82.19 | | O |
| ANISOU | 3338 | O | ILE | P | 266 | 14252 | 9177 | 7798 | −2465 | 1709 | −505 | O |
| ATOM | 3339 | CB | ILE | P | 266 | −6.617 | 19.675 | −14.518 | 1.00 | 78.06 | | C |
| ANISOU | 3339 | CB | ILE | P | 266 | 13719 | 8232 | 7709 | −2518 | 2455 | 147 | C |
| ATOM | 3340 | CG1 | ILE | P | 266 | −5.546 | 19.003 | −13.661 | 1.00 | 80.88 | | C |
| ANISOU | 3340 | CG1 | ILE | P | 266 | 13614 | 8689 | 8428 | −2834 | 2367 | 295 | C |
| ATOM | 3341 | CG2 | ILE | P | 266 | −5.970 | 20.572 | −15.555 | 1.00 | 79.39 | | C |
| ANISOU | 3341 | CG2 | ILE | P | 266 | 14092 | 8085 | 7986 | −2562 | 2711 | 381 | C |
| ATOM | 3342 | CD1 | ILE | P | 266 | −4.627 | 18.100 | −14.447 | 1.00 | 81.89 | | C |
| ANISOU | 3342 | CD1 | ILE | P | 266 | 13428 | 8887 | 8799 | −2850 | 2692 | 565 | C |
| ATOM | 3343 | N | THR | P | 267 | −9.499 | 19.019 | −13.812 | 1.00 | 80.38 | | N |
| ANISOU | 3343 | N | THR | P | 267 | 14300 | 9006 | 7236 | −1969 | 2120 | −540 | N |
| ATOM | 3344 | CA | THR | P | 267 | −10.486 | 18.064 | −13.339 | 1.00 | 74.40 | | C |
| ANISOU | 3344 | CA | THR | P | 267 | 13432 | 8579 | 6258 | −1823 | 2026 | −796 | C |
| ATOM | 3345 | C | THR | P | 267 | −9.782 | 16.825 | −12.802 | 1.00 | 74.01 | | C |
| ANISOU | 3345 | C | THR | P | 267 | 12977 | 8689 | 6456 | −2022 | 2059 | −671 | C |
| ATOM | 3346 | O | THR | P | 267 | −8.683 | 16.485 | −13.251 | 1.00 | 67.48 | | O |
| ANISOU | 3346 | O | THR | P | 267 | 11949 | 7775 | 5917 | −2152 | 2249 | −391 | O |
| ATOM | 3347 | CB | THR | P | 267 | −11.443 | 17.644 | −14.465 | 1.00 | 73.99 | | C |
| ANISOU | 3347 | CB | THR | P | 267 | 13523 | 8679 | 5910 | −1462 | 2221 | −933 | C |
| ATOM | 3348 | OG1 | THR | P | 267 | −10.701 | 17.004 | −15.514 | 1.00 | 60.56 | | O |
| ANISOU | 3348 | OG1 | THR | P | 267 | 11695 | 6938 | 4375 | −1444 | 2539 | −686 | O |
| ATOM | 3349 | CG2 | THR | P | 267 | −12.174 | 18.855 | −15.024 | 1.00 | 77.11 | | C |
| ANISOU | 3349 | CG2 | THR | P | 267 | 14340 | 8931 | 6028 | −1209 | 2179 | −1063 | C |
| ATOM | 3350 | N | ASN | P | 268 | −10.417 | 16.159 | −11.840 | 1.00 | 59.47 | | N |
| ANISOU | 3350 | N | ASN | P | 268 | 11025 | 7080 | 4491 | −2033 | 1884 | −876 | N |
| ATOM | 3351 | CA | ASN | P | 268 | −9.888 | 14.920 | −11.286 | 1.00 | 48.08 | | C |
| ANISOU | 3351 | CA | ASN | P | 268 | 9252 | 5793 | 3225 | −2179 | 1909 | −783 | C |
| ATOM | 3352 | C | ASN | P | 268 | −9.596 | 13.891 | −12.375 | 1.00 | 55.47 | | C |
| ANISOU | 3352 | C | ASN | P | 268 | 10056 | 6797 | 4225 | −2062 | 2237 | −637 | C |
| ATOM | 3353 | O | ASN | P | 268 | −8.630 | 13.133 | −12.273 | 1.00 | 54.86 | | O |
| ANISOU | 3353 | O | ASN | P | 268 | 9713 | 6728 | 4405 | −2194 | 2331 | −421 | O |
| ATOM | 3354 | CB | ASN | P | 268 | −10.855 | 14.328 | −10.257 | 1.00 | 53.90 | | C |
| ANISOU | 3354 | CB | ASN | P | 268 | 9967 | 6772 | 3740 | −2156 | 1726 | −1056 | C |
| ATOM | 3355 | CG | ASN | P | 268 | −10.927 | 15.144 | −8.978 | 1.00 | 58.38 | | C |
| ANISOU | 3355 | CG | ASN | P | 268 | 10612 | 7284 | 4285 | −2303 | 1394 | −1166 | C |
| ATOM | 3356 | OD1 | ASN | P | 268 | −10.158 | 16.083 | −8.779 | 1.00 | 75.62 | | O |
| ANISOU | 3356 | OD1 | ASN | P | 268 | 12839 | 9243 | 6652 | −2460 | 1280 | −1032 | O |
| ATOM | 3357 | ND2 | ASN | P | 268 | −11.852 | 14.783 | −8.101 | 1.00 | 47.61 | | N |
| ANISOU | 3357 | ND2 | ASN | P | 268 | 9272 | 6124 | 2693 | −2264 | 1246 | −1417 | N |
| ATOM | 3358 | N | ASP | P | 269 | −10.431 | 13.863 | −13.413 | 1.00 | 50.59 | | N |
| ANISOU | 3358 | N | ASP | P | 269 | 9628 | 6233 | 3359 | −1795 | 2401 | −762 | N |
| ATOM | 3359 | CA | ASP | P | 269 | −10.226 | 12.943 | −14.528 | 1.00 | 56.37 | | C |
| ANISOU | 3359 | CA | ASP | P | 269 | 10286 | 7018 | 4115 | −1657 | 2711 | −646 | C |
| ATOM | 3360 | C | ASP | P | 269 | −8.905 | 13.221 | −15.234 | 1.00 | 59.80 | | C |
| ANISOU | 3360 | C | ASP | P | 269 | 10635 | 7235 | 4851 | −1743 | 2918 | −294 | C |
| ATOM | 3361 | O | ASP | P | 269 | −8.203 | 12.299 | −15.639 | 1.00 | 62.89 | | O |
| ANISOU | 3361 | O | ASP | P | 269 | 10824 | 7662 | 5411 | −1754 | 3128 | −108 | O |
| ATOM | 3362 | CB | ASP | P | 269 | −11.370 | 13.039 | −15.538 | 1.00 | 71.42 | | C |
| ANISOU | 3362 | CB | ASP | P | 269 | 12391 | 8940 | 5804 | −1348 | 2747 | −844 | C |
| ATOM | 3363 | CG | ASP | P | 269 | −12.648 | 12.395 | −15.041 | 1.00 | 84.67 | | C |
| ANISOU | 3363 | CG | ASP | P | 269 | 13937 | 10746 | 7487 | −1286 | 2495 | −1096 | C |
| ATOM | 3364 | OD1 | ASP | P | 269 | −12.604 | 11.690 | −14.010 | 1.00 | 92.63 | | O |
| ANISOU | 3364 | OD1 | ASP | P | 269 | 14755 | 11867 | 8574 | −1464 | 2388 | −1136 | O |
| ATOM | 3365 | OD2 | ASP | P | 269 | −13.698 | 12.591 | −15.688 | 1.00 | 83.85 | | O |
| ANISOU | 3365 | OD2 | ASP | P | 269 | 13907 | 10648 | 7302 | −1064 | 2423 | −1224 | O |
| ATOM | 3366 | N | GLN | P | 270 | −8.573 | 14.497 | −15.390 | 1.00 | 64.57 | | N |
| ANISOU | 3366 | N | GLN | P | 270 | 11405 | 7611 | 5517 | −1803 | 2873 | −206 | N |
| ATOM | 3367 | CA | GLN | P | 270 | −7.335 | 14.872 | −16.063 | 1.00 | 71.87 | | C |
| ANISOU | 3367 | CA | GLN | P | 270 | 12255 | 8322 | 6732 | −1916 | 3088 | 124 | C |
| ATOM | 3368 | C | GLN | P | 270 | −6.111 | 14.487 | −15.232 | 1.00 | 66.25 | | C |
| ANISOU | 3368 | C | GLN | P | 270 | 11165 | 7611 | 6395 | −2220 | 3022 | 333 | C |
| ATOM | 3369 | O | GLN | P | 270 | −5.112 | 14.014 | −15.771 | 1.00 | 63.28 | | O |
| ANISOU | 3369 | O | GLN | P | 270 | 10569 | 7210 | 6264 | −2269 | 3258 | 593 | O |
| ATOM | 3370 | CB | GLN | P | 270 | −7.334 | 16.364 | −16.420 | 1.00 | 77.84 | | C |
| ANISOU | 3370 | CB | GLN | P | 270 | 13324 | 8807 | 7444 | −1924 | 3063 | 156 | C |
| ATOM | 3371 | CG | GLN | P | 270 | −8.148 | 16.691 | −17.673 | 1.00 | 80.33 | | C |
| ANISOU | 3371 | CG | GLN | P | 270 | 13996 | 9079 | 7446 | −1579 | 3248 | 70 | C |
| ATOM | 3372 | CD | GLN | P | 270 | −8.616 | 18.136 | −17.723 | 1.00 | 83.13 | | C |
| ANISOU | 3372 | CD | GLN | P | 270 | 14748 | 9212 | 7627 | −1516 | 3120 | −21 | C |
| ATOM | 3373 | OE1 | GLN | P | 270 | −8.413 | 18.901 | −16.780 | 1.00 | 84.81 | | O |
| ANISOU | 3373 | OE1 | GLN | P | 270 | 14984 | 9302 | 7938 | −1737 | 2880 | −48 | O |
| ATOM | 3374 | NE2 | GLN | P | 270 | −9.256 | 18.512 | −18.824 | 1.00 | 79.56 | | N |
| ANISOU | 3374 | NE2 | GLN | P | 270 | 14633 | 8701 | 6894 | −1195 | 3272 | −78 | N |
| ATOM | 3375 | N | LYS | P | 271 | −6.196 | 14.678 | −13.918 | 1.00 | 60.48 | | N |
| ANISOU | 3375 | N | LYS | P | 271 | 10357 | 6924 | 5697 | −2403 | 2697 | 213 | N |
| ATOM | 3376 | CA | LYS | P | 271 | −5.116 | 14.283 | −13.018 | 1.00 | 55.55 | | C |
| ANISOU | 3376 | CA | LYS | P | 271 | 9378 | 6334 | 5394 | −2666 | 2580 | 374 | C |
| ATOM | 3377 | C | LYS | P | 271 | −4.934 | 12.773 | −13.079 | 1.00 | 57.38 | | C |
| ANISOU | 3377 | C | LYS | P | 271 | 9365 | 6774 | 5663 | −2571 | 2725 | 433 | C |
| ATOM | 3378 | O | LYS | P | 271 | −3.825 | 12.271 | −13.244 | 1.00 | 62.74 | | O |
| ANISOU | 3378 | O | LYS | P | 271 | 9755 | 7458 | 6624 | −2654 | 2863 | 681 | O |
| ATOM | 3379 | CB | LYS | P | 271 | −5.428 | 14.700 | −11.578 | 1.00 | 63.38 | | C |
| ANISOU | 3379 | CB | LYS | P | 271 | 10389 | 7354 | 6338 | −2828 | 2186 | 190 | C |
| ATOM | 3380 | CG | LYS | P | 271 | −5.798 | 16.165 | −11.399 | 1.00 | 71.40 | | C |
| ANISOU | 3380 | CG | LYS | P | 271 | 11709 | 8164 | 7255 | −2891 | 2007 | 77 | C |
| ATOM | 3381 | CD | LYS | P | 271 | −5.976 | 16.508 | −9.923 | 1.00 | 73.41 | | C |
| ANISOU | 3381 | CD | LYS | P | 271 | 11962 | 8448 | 7481 | −3058 | 1618 | −89 | C |
| ATOM | 3382 | CE | LYS | P | 271 | −6.348 | 17.972 | −9.729 | 1.00 | 79.73 | | C |
| ANISOU | 3382 | CE | LYS | P | 271 | 13100 | 9024 | 8170 | −3106 | 1436 | −210 | C |
| ATOM | 3383 | NZ | LYS | P | 271 | −6.484 | 18.328 | −8.289 | 1.00 | 81.22 | | N |
| ANISOU | 3383 | NZ | LYS | P | 271 | 13306 | 9231 | 8324 | −3263 | 1055 | −373 | N |
| ATOM | 3384 | N | LYS | P | 272 | −6.045 | 12.061 | −12.936 | 1.00 | 59.97 | | N |
| ANISOU | 3384 | N | LYS | P | 272 | 9815 | 7272 | 5698 | −2396 | 2694 | 196 | N |
| ATOM | 3385 | CA | LYS | P | 272 | −6.067 | 10.609 | −13.005 | 1.00 | 64.48 | | C |
| ANISOU | 3385 | CA | LYS | P | 272 | 10236 | 8018 | 6247 | −2295 | 2835 | 211 | C |
| ATOM | 3386 | C | LYS | P | 272 | −5.540 | 10.125 | −14.347 | 1.00 | 65.65 | | C |
| ANISOU | 3386 | C | LYS | P | 272 | 10337 | 8126 | 6481 | −2144 | 3203 | 414 | C |
| ATOM | 3387 | O | LYS | P | 272 | −4.759 | 9.179 | −14.420 | 1.00 | 73.32 | | O |
| ANISOU | 3387 | O | LYS | P | 272 | 11076 | 9153 | 7629 | −2143 | 3344 | 594 | O |
| ATOM | 3388 | CB | LYS | P | 272 | −7.498 | 10.111 | −12.791 | 1.00 | 71.71 | | C |
| ANISOU | 3388 | CB | LYS | P | 272 | 11343 | 9099 | 6804 | −2148 | 2769 | −110 | C |
| ATOM | 3389 | CG | LYS | P | 272 | −7.654 | 8.607 | −12.842 | 1.00 | 69.02 | | C |
| ANISOU | 3389 | CG | LYS | P | 272 | 10904 | 8914 | 6405 | −2062 | 2916 | −129 | C |
| ATOM | 3390 | CD | LYS | P | 272 | −9.070 | 8.192 | −12.486 | 1.00 | 63.72 | | C |
| ANISOU | 3390 | CD | LYS | P | 272 | 10380 | 8374 | 5458 | −1978 | 2784 | −475 | C |
| ATOM | 3391 | CE | LYS | P | 272 | −9.260 | 6.706 | −12.712 | 1.00 | 72.38 | | C |
| ANISOU | 3391 | CE | LYS | P | 272 | 11382 | 9479 | 6638 | −1889 | 2842 | −522 | C |
| ATOM | 3392 | NZ | LYS | P | 272 | −10.652 | 6.276 | −12.439 | 1.00 | 78.98 | | N |
| ANISOU | 3392 | NZ | LYS | P | 272 | 12280 | 10361 | 7368 | −1861 | 2666 | −820 | N |
| ATOM | 3393 | N | LEU | P | 273 | −5.982 | 10.782 | −15.412 | 1.00 | 72.34 | | N |
| ANISOU | 3393 | N | LEU | P | 273 | 11427 | 8876 | 7183 | −1992 | 3357 | 382 | N |
| ATOM | 3394 | CA | LEU | P | 273 | −5.528 | 10.465 | −16.757 | 1.00 | 79.75 | | C |
| ANISOU | 3394 | CA | LEU | P | 273 | 12374 | 9757 | 8168 | −1828 | 3715 | 570 | C |
| ATOM | 3395 | C | LEU | P | 273 | −4.010 | 10.595 | −16.852 | 1.00 | 79.24 | | C |
| ANISOU | 3395 | C | LEU | P | 273 | 12026 | 9589 | 8493 | −1996 | 3846 | 913 | C |
| ATOM | 3396 | O | LEU | P | 273 | −3.330 | 9.710 | −17.371 | 1.00 | 75.30 | | O |
| ANISOU | 3396 | O | LEU | P | 273 | 11345 | 9139 | 8126 | −1917 | 4088 | 1097 | O |
| ATOM | 3397 | CB | LEU | P | 273 | −6.200 | 11.395 | −17.767 | 1.00 | 94.04 | | C |
| ANISOU | 3397 | CB | LEU | P | 273 | 14528 | 11450 | 9753 | −1649 | 3815 | 487 | C |
| ATOM | 3398 | CG | LEU | P | 273 | −5.770 | 11.277 | −19.227 | 1.00 | 113.16 | | C |
| ANISOU | 3398 | CG | LEU | P | 273 | 17028 | 13782 | 12187 | −1462 | 4190 | 682 | C |
| ATOM | 3399 | CD1 | LEU | P | 273 | −6.239 | 9.956 | −19.811 | 1.00 | 112.29 | | C |
| ANISOU | 3399 | CD1 | LEU | P | 273 | 16900 | 13764 | 12001 | −1203 | 4252 | 540 | C |
| ATOM | 3400 | CD2 | LEU | P | 273 | −6.307 | 12.451 | −20.037 | 1.00 | 120.74 | | C |
| ANISOU | 3400 | CD2 | LEU | P | 273 | 18353 | 14581 | 12941 | −1316 | 4239 | 626 | C |
| ATOM | 3401 | N | MET | P | 274 | −3.485 | 11.700 | −16.334 | 1.00 | 79.04 | | N |
| ANISOU | 3401 | N | MET | P | 274 | 11957 | 9425 | 8650 | −2231 | 3683 | 989 | N |
| ATOM | 3402 | CA | MET | P | 274 | −2.059 | 11.987 | −16.418 | 1.00 | 74.78 | | C |
| ANISOU | 3402 | CA | MET | P | 274 | 11127 | 8792 | 8495 | −2431 | 3795 | 1296 | C |
| ATOM | 3403 | C | MET | P | 274 | −1.203 | 11.155 | −15.457 | 1.00 | 65.60 | | C |
| ANISOU | 3403 | C | MET | P | 274 | 9560 | 7778 | 7586 | −2574 | 3665 | 1398 | C |
| ATOM | 3404 | O | MET | P | 274 | −0.024 | 10.928 | −15.710 | 1.00 | 74.49 | | O |
| ANISOU | 3404 | O | MET | P | 274 | 10378 | 8910 | 9013 | −2651 | 3830 | 1656 | O |
| ATOM | 3405 | CB | MET | P | 274 | −1.801 | 13.479 | −16.198 | 1.00 | 74.94 | | C |
| ANISOU | 3405 | CB | MET | P | 274 | 11256 | 8595 | 8623 | −2663 | 3660 | 1326 | C |
| ATOM | 3406 | CG | MET | P | 274 | −2.416 | 14.374 | −17.260 | 1.00 | 74.28 | | C |
| ANISOU | 3406 | CG | MET | P | 274 | 11579 | 8324 | 8320 | −2509 | 3832 | 1289 | C |
| ATOM | 3407 | SD | MET | P | 274 | −2.167 | 16.114 | −16.892 | 1.00 | 88.02 | | S |
| ANISOU | 3407 | SD | MET | P | 274 | 13510 | 9770 | 10163 | −2789 | 3656 | 1307 | S |
| ATOM | 3408 | CE | MET | P | 274 | −0.385 | 16.157 | −16.707 | 1.00 | 90.50 | | C |
| ANISOU | 3408 | CE | MET | P | 274 | 13341 | 10039 | 11004 | −3149 | 3763 | 1647 | C |
| ATOM | 3409 | N | SER | P | 275 | −1.786 | 10.698 | −14.357 | 1.00 | 58.19 | | N |
| ANISOU | 3409 | N | SER | P | 275 | 8622 | 6967 | 6520 | −2596 | 3377 | 1198 | N |
| ATOM | 3410 | CA | SER | P | 275 | −1.010 | 9.947 | −13.374 | 1.00 | 64.47 | | C |
| ANISOU | 3410 | CA | SER | P | 275 | 9078 | 7895 | 7523 | −2708 | 3223 | 1286 | C |
| ATOM | 3411 | C | SER | P | 275 | −0.910 | 8.458 | −13.721 | 1.00 | 74.91 | | C |
| ANISOU | 3411 | C | SER | P | 275 | 10295 | 9363 | 8806 | −2489 | 3436 | 1353 | C |
| ATOM | 3412 | O | SER | P | 275 | 0.101 | 7.817 | −13.440 | 1.00 | 72.80 | | O |
| ANISOU | 3412 | O | SER | P | 275 | 9708 | 9176 | 8775 | −2514 | 3463 | 1541 | O |
| ATOM | 3413 | CB | SER | P | 275 | −1.561 | 10.161 | −11.957 | 1.00 | 60.08 | | C |
| ANISOU | 3413 | CB | SER | P | 275 | 8580 | 7392 | 6856 | −2841 | 2817 | 1068 | C |
| ATOM | 3414 | OG | SER | P | 275 | −2.883 | 9.673 | −11.829 | 1.00 | 70.03 | | O |
| ANISOU | 3414 | OG | SER | P | 275 | 10112 | 8736 | 7759 | −2675 | 2778 | 807 | O |
| ATOM | 3415 | N | ASN | P | 276 | −1.963 | 7.919 | −14.334 | 1.00 | 83.21 | | N |
| ANISOU | 3415 | N | ASN | P | 276 | 11620 | 10445 | 9551 | −2269 | 3580 | 1189 | N |
| ATOM | 3416 | CA | ASN | P | 276 | −1.973 | 6.536 | −14.813 | 1.00 | 85.57 | | C |
| ANISOU | 3416 | CA | ASN | P | 276 | 11892 | 10842 | 9777 | −2054 | 3812 | 1230 | C |
| ATOM | 3417 | C | ASN | P | 276 | −1.096 | 6.359 | −16.042 | 1.00 | 96.38 | | C |
| ANISOU | 3417 | C | ASN | P | 276 | 13146 | 12159 | 11314 | −1929 | 4178 | 1491 | C |
| ATOM | 3418 | O | ASN | P | 276 | −0.144 | 5.567 | −16.049 | 1.00 | 92.15 | | O |
| ANISOU | 3418 | O | ASN | P | 276 | 12348 | 11689 | 10978 | −1880 | 4301 | 1694 | O |
| ATOM | 3419 | CB | ASN | P | 276 | −3.381 | 6.118 | −15.237 | 1.00 | 75.58 | | C |
| ANISOU | 3419 | CB | ASN | P | 276 | 10961 | 9605 | 8152 | −1863 | 3840 | 940 | C |
| ATOM | 3420 | CG | ASN | P | 276 | −4.332 | 5.986 | −14.084 | 1.00 | 72.43 | | C |
| ANISOU | 3420 | CG | ASN | P | 276 | 10672 | 9306 | 7544 | −1958 | 3565 | 685 | C |
| ATOM | 3421 | OD1 | ASN | P | 276 | −3.988 | 5.453 | −13.032 | 1.00 | 74.52 | | O |
| ANISOU | 3421 | OD1 | ASN | P | 276 | 10786 | 9642 | 7885 | −2060 | 3406 | 716 | O |
| ATOM | 3422 | ND2 | ASN | P | 276 | −5.554 | 6.460 | −14.280 | 1.00 | 76.67 | | N |
| ANISOU | 3422 | ND2 | ASN | P | 276 | 11472 | 9834 | 7825 | −1890 | 3468 | 404 | N |
| ATOM | 3423 | N | ASN | P | 277 | −1.460 | 7.108 | −17.082 | 1.00 | 109.71 | | N |
| ANISOU | 3423 | N | ASN | P | 277 | 15053 | 13728 | 12903 | −1847 | 4339 | 1470 | N |
| ATOM | 3424 | CA | ASN | P | 277 | −0.906 | 6.972 | −18.430 | 1.00 | 126.53 | | C |
| ANISOU | 3424 | CA | ASN | P | 277 | 17182 | 15771 | 15122 | −1646 | 4669 | 1637 | C |
| ATOM | 3425 | C | ASN | P | 277 | 0.332 | 6.120 | −18.713 | 1.00 | 157.12 | | C |
| ANISOU | 3425 | C | ASN | P | 277 | 20737 | 19702 | 19260 | −1569 | 4888 | 1908 | C |
| ATOM | 3426 | O | ASN | P | 277 | 0.450 | 5.458 | −19.705 | 1.00 | 165.30 | | O |
| ANISOU | 3426 | O | ASN | P | 277 | 21836 | 20704 | 20266 | −1305 | 5122 | 1951 | O |
| ATOM | 3427 | CB | ASN | P | 277 | −0.925 | 8.285 | −19.231 | 1.00 | 126.96 | | C |
| ANISOU | 3427 | CB | ASN | P | 277 | 17414 | 15664 | 15160 | −1697 | 4809 | 1710 | C |
| ATOM | 3428 | CG | ASN | P | 277 | 0.019 | 9.368 | −18.832 | 1.00 | 135.53 | | C |
| ANISOU | 3428 | CG | ASN | P | 277 | 18295 | 16661 | 16538 | −2011 | 4771 | 1919 | C |
| ATOM | 3429 | OD1 | ASN | P | 277 | −0.273 | 10.174 | −17.959 | 1.00 | 144.85 | | O |
| ANISOU | 3429 | OD1 | ASN | P | 277 | 19523 | 17793 | 17721 | −2213 | 4463 | 1791 | O |
| ATOM | 3430 | ND2 | ASN | P | 277 | 0.991 | 9.583 | −19.696 | 1.00 | 136.64 | | N |
| ANISOU | 3430 | ND2 | ASN | P | 277 | 18299 | 16728 | 16891 | −2014 | 5086 | 2191 | N |
| END | | | | | | | | | | | | |

The Ramachandran plot shows 95.6% of all residues in favored regions and 99.3% of all residues in allowed regions. All structural images were created using PyMol (Delano Scientific, http://www.pymol.org).

f. Cloning, expression and purification of RSV F₀ Fd, also referred to as RSV F0 Fd. A codon-optimized DNA fragment encoding amino acid residues 1-513 of the RSV F protein strain A2 with mutations R106Q, R109S, R135S and R136S was synthesized by GeneArt with a 3′ fragment encoding the residues SAIGGYIPEA PRDGQAYVRK DGEWVLLSTF LGGIEGRHHH HHH, also denoted herein as SEQ ID NO:15). This gene was cloned into a variant of the pHLSec mammalian expression vector (Aricescu A R, et. al., 2006, Acta Crystallogr. D Biol. Crystallogr. 62, 1243-1250) and protein was expressed using the 293Freestyle expression system as described above for the motavizumab IgG expression. Protein was purified from the supernatant using Ni-NTA resin (Qiagen, Venlo, the Netherlands) followed by gel filtration on a SUPEROSE™6 column with a running buffer of 2 mM Tris-HCl pH 7.5, 150 mM NaCl. The peak corresponding to a trimer was pooled, concentrated and stored at 4° C.

g. RSV F₀ Fd cross-linking and immunoprecipitation. RSV F₀ Fd (5 μg, 0.2 μM) in PBS was incubated with glutaraldehyde at concentrations of 0, 1, and 10 mM for 5 min at room temperature. Glycine was added to a final concentration of 100 mM to quench the reaction. The cross-linked and control proteins were incubated with 5 μg of motavizumab IgG for 30 min at room temperature. 20 μl of a Protein A agarose slurry (Pierce) was added and incubated for 90 min at room temperature. The resin was centrifuged, washed with PBS containing Tween 20, and then boiled in reducing SDS-PAGE loading buffer.

### Example 2

### Three-dimensional structure of RSV F protein and 101F antibody

This Example describes the crystallization and determination of the 3-dimensional structure of a complex between the 101F antibody and the 15-residue RSV fusion (F) peptide corresponding to amino acids 422-436 of the F protein (i.e., STASNKNRGI IKTFS, also denoted herein as SEQ ID NO:3) that includes the binding domain of 101F.

Recombinant 101F IgG and a peptide comprising the 101F binding domain were combined and the resultant complex submitted to crystallization and analysis as follows.

a. Cloning, expression and purification of 101F IgG: Two DNA fragments encoding the variable heavy and light chains of 101F with signal sequences were synthesized by GeneArt and cloned in-frame into mammalian expression vectors containing mouse IgG1 heavy and light constant domains, respectively. The amino acid sequence of the variable heavy chain of 101F is as follows: QVTLKESGPG ILQPSQTLSL TCSFSGFSLS TSGMGVSWIR QPSGKGLEWL AHIYWDDDKR YNPSLKSRLT ISKDTSRNQV FLKITSVDTA DTATYYCARL YGFTYGFAYW GQGTLVTVSA, also denoted herein as SEQ ID NO:7. The amino acid sequence of the variable light chain of 101F is as follows: DIVLTQSPAS LAVSLGQRAT IFCRASQSVD YNGISYMHWF QQKPGQPPKL LIYAASNPES GIPARFTGSG SGTDFTLNIH PVEEEDAATY YCQQIIEDPW TFGGGTKLEI K, also denoted herein as SEQ ID NO:8.

Both vectors were co-transfected at a 1:1 ratio into HEK293F cells (Invitrogen) in serum-free 293Freestyle medium (Invitrogen). After 3 hours, valproic acid (Sigma) was added to 4 mM final concentration. Expression lasted for five days at 37° C. with 10% CO₂ and shaking at 125 rpm in disposable flasks. The supernatant was collected, filtered, and passed over 5 ml of Protein G agarose resin (Pierce). After washing with several column volumes of phosphate-buffered saline (PBS), the resin was eluted with 15 ml of IgG Elution Buffer (Pierce) and immediately neutralized with 1 M Tris pH 8.0. The eluted protein was dialyzed against PBS and stored at 4° C.

b. Digestion and purification of 101F Fab fragments. The purified 101F IgG protein was reduced with 100 mM dithiothreitol at 37° C. for 1 hour and then alkylated with 2 mM iodoacetamide for 48 hours at 4° C. Ten ml of reduced and alkylated IgG in PBS at 1.5 mg/ml was combined with 0.275 ODs of Ficin (Sigma), 20 mM L-cysteine, 1 mM EDTA and incubated at 37° C. for 1 hour. The reaction was quenched by the addition of iodoacetamide to 40 mM final concentration. To remove the Fc fragments from the Fab fragments, the quenched reaction was passed over 1 ml of Protein A agarose. The Fab-containing flow through was further purified over an S200 gel filtration column and concentrated aliquots were stored frozen at −80° C.

c. Protein crystallization and data collection. A peptide with the sequence STASNKNRGI IKTFS (SEQ ID NO:3), corresponding to the originally identified 101F epitope of CTASNKNRGI IKTFS (residues 422-436 of RSV F protein), also denoted herein as SEQ ID NO:10, was synthesized by American Peptide with an acetylated N-terminus and an amidated C-terminus. A five-fold molar excess of peptide was incubated with 101F Fab at 22° C. for 1.5 hours and then concentrated to give an 8.3 mg/ml solution of Fab/peptide complex. Crystals were grown by the vapor diffusion method in sitting drops at 20° C. by mixing 1 μl of protein complex with 1 μl of reservoir solution (15% (w/v) PEG 4000, 0.2 M lithium sulfate, 0.1 M Tris pH 8.5). These crystals were flash frozen in liquid nitrogen in 20% (w/v) PEG 4000, 0.2M lithium sulfate, 0.1M Tris pH 8.5 and 15% (v/v) 2R,3R-butanediol. Data were collected at a wavelength of 0.82656 Å at the SER-CAT beamline ID-22 (Advanced Photon Source, Argonne National Laboratory).

The structure of the 101F/peptide complex was determined and a model built and refined using a method similar to that described in Example 1. Final data collection and refinement statistics are presented in Table 4.

The atomic coordinates for the complex between the 15-residue F peptide and 101F antibody are indicated in PDB acc code 3O41. The atomic coordinates for the peptide portion of the complex are indicated below in Table 7.

TABLE 7
Atomic coordinates of the 101F binding peptide

| | | | | | | | | | | | | |
|---|---|---|---|---|---|---|---|---|---|---|---|---|
| ATOM | 6696 | O | LYS | P | 427 | −17.126 | 3.607 | 14.869 | 1.00 | 82.36 | | O |
| ANISOU | 6696 | O | LYS | P | 427 | 11025 | 9697 | 10570 | 618 | 188 | 376 | O |
| ATOM | 6697 | N | LYS | P | 427 | −17.615 | 3.567 | 18.200 | 1.00 | 90.82 | | N |
| ANISOU | 6697 | N | LYS | P | 427 | 12312 | 10602 | 11594 | 727 | 198 | 440 | N |
| ATOM | 6698 | CA | LYS | P | 427 | −18.312 | 3.323 | 16.942 | 1.00 | 89.25 | | C |
| ANISOU | 6698 | CA | LYS | P | 427 | 12014 | 10493 | 11404 | 684 | 241 | 396 | C |
| ATOM | 6699 | C | LYS | P | 427 | −17.352 | 2.880 | 15.838 | 1.00 | 83.29 | | C |
| ANISOU | 6699 | C | LYS | P | 427 | 11189 | 9761 | 10696 | 633 | 229 | 378 | C |
| ATOM | 6700 | CB | LYS | P | 427 | −19.092 | 4.566 | 16.507 | 1.00 | 92.06 | | C |
| ANISOU | 6700 | CB | LYS | P | 427 | 12357 | 10914 | 11709 | 704 | 213 | 400 | C |
| ATOM | 6701 | CG | LYS | P | 427 | −18.301 | 5.861 | 16.568 | 1.00 | 92.91 | | C |
| ANISOU | 6701 | CG | LYS | P | 427 | 12507 | 11004 | 11790 | 733 | 117 | 442 | C |
| ATOM | 6702 | CD | LYS | P | 427 | −19.182 | 7.057 | 16.249 | 1.00 | 93.83 | | C |
| ANISOU | 6702 | CD | LYS | P | 427 | 12615 | 11181 | 11854 | 756 | 94 | 446 | C |
| ATOM | 6703 | CE | LYS | P | 427 | −18.427 | 8.362 | 16.431 | 1.00 | 95.87 | | C |
| ANISOU | 6703 | CE | LYS | P | 427 | 12924 | 11417 | 12084 | 790 | −2 | 490 | C |
| ATOM | 6704 | NZ | LYS | P | 427 | −19.183 | 9.529 | 15.890 | 1.00 | 97.27 | | N |
| ANISOU | 6704 | NZ | LYS | P | 427 | 13079 | 11662 | 12216 | 804 | −30 | 491 | N |
| ATOM | 6705 | O | ASN | P | 428 | −17.432 | 0.121 | 13.533 | 1.00 | 63.07 | | O |
| ANISOU | 6705 | O | ASN | P | 428 | 8415 | 7298 | 8250 | 494 | 373 | 266 | O |
| ATOM | 6706 | N | ASN | P | 428 | −16.796 | 1.681 | 15.999 | 1.00 | 77.41 | | N |
| ANISOU | 6706 | N | ASN | P | 428 | 10437 | 8975 | 10000 | 607 | 264 | 364 | N |
| ATOM | 6707 | CA | ASN | P | 428 | −15.846 | 1.113 | 15.044 | 1.00 | 72.01 | | C |
| ANISOU | 6707 | CA | ASN | P | 428 | 9689 | 8305 | 9366 | 558 | 258 | 347 | C |
| ATOM | 6708 | C | ASN | P | 428 | −16.459 | 0.867 | 13.665 | 1.00 | 62.88 | | C |
| ANISOU | 6708 | C | ASN | P | 428 | 8429 | 7243 | 8221 | 511 | 299 | 301 | C |
| ATOM | 6709 | CB | ASN | P | 428 | −15.262 | −0.194 | 15.597 | 1.00 | 75.94 | | C |
| ANISOU | 6709 | CB | ASN | P | 428 | 10204 | 8738 | 9913 | 541 | 296 | 340 | C |
| ATOM | 6710 | CG | ASN | P | 428 | −14.397 | −0.927 | 14.583 | 1.00 | 78.67 | | C |
| ANISOU | 6710 | CG | ASN | P | 428 | 10478 | 9101 | 10312 | 488 | 302 | 315 | C |
| ATOM | 6711 | OD1 | ASN | P | 428 | −13.646 | −0.310 | 13.824 | 1.00 | 78.53 | | O |
| ANISOU | 6711 | OD1 | ASN | P | 428 | 10433 | 9107 | 10298 | 475 | 246 | 324 | O |
| ATOM | 6712 | ND2 | ASN | P | 428 | −14.493 | −2.255 | 14.574 | 1.00 | 79.70 | | N |
| ANISOU | 6712 | ND2 | ASN | P | 428 | 10577 | 9219 | 10484 | 456 | 368 | 283 | N |
| ATOM | 6713 | O | ARG | P | 429 | −16.017 | −0.008 | 9.350 | 1.00 | 42.31 | | O |
| ANISOU | 6713 | O | ARG | P | 429 | 5527 | 4851 | 5699 | 350 | 340 | 193 | O |
| ATOM | 6714 | N | ARG | P | 429 | −15.878 | 1.492 | 12.642 | 1.00 | 51.34 | | N |
| ANISOU | 6714 | N | ARG | P | 429 | 6920 | 5825 | 6761 | 490 | 250 | 301 | N |
| ATOM | 6715 | CA | ARG | P | 429 | −16.374 | 1.375 | 11.272 | 1.00 | 46.50 | | C |
| ANISOU | 6715 | CA | ARG | P | 429 | 6208 | 5304 | 6157 | 445 | 280 | 260 | C |
| ATOM | 6716 | C | ARG | P | 429 | −15.659 | 0.274 | 10.490 | 1.00 | 43.56 | | C |
| ANISOU | 6716 | C | ARG | P | 429 | 5770 | 4938 | 5843 | 392 | 309 | 230 | C |
| ATOM | 6717 | CB | ARG | P | 429 | −16.163 | 2.690 | 10.515 | 1.00 | 44.81 | | C |
| ANISOU | 6717 | CB | ARG | P | 429 | 5973 | 5139 | 5912 | 450 | 211 | 277 | C |
| ATOM | 6718 | CG | ARG | P | 429 | −17.099 | 3.817 | 10.872 | 1.00 | 45.89 | | C |
| ANISOU | 6718 | CG | ARG | P | 429 | 6145 | 5300 | 5990 | 492 | 190 | 294 | C |
| ATOM | 6719 | CD | ARG | P | 429 | −18.397 | 3.691 | 10.094 | 1.00 | 45.47 | | C |
| ANISOU | 6719 | CD | ARG | P | 429 | 6021 | 5331 | 5924 | 469 | 249 | 254 | C |
| ATOM | 6720 | NE | ARG | P | 429 | −19.454 | 3.112 | 10.897 | 1.00 | 43.13 | | N |
| ANISOU | 6720 | NE | ARG | P | 429 | 5752 | 5019 | 5617 | 488 | 315 | 242 | N |
| ATOM | 6721 | CZ | ARG | P | 429 | −20.614 | 2.677 | 10.413 | 1.00 | 45.12 | | C |
| ANISOU | 6721 | CZ | ARG | P | 429 | 5947 | 5330 | 5866 | 467 | 382 | 204 | C |
| ATOM | 6722 | NH1 | ARG | P | 429 | −20.876 | 2.723 | 9.114 | 1.00 | 37.74 | | N |
| ANISOU | 6722 | NH1 | ARG | P | 429 | 4925 | 4475 | 4939 | 426 | 393 | 173 | N |
| ATOM | 6723 | NH2 | ARG | P | 429 | −21.514 | 2.173 | 11.239 | 1.00 | 49.71 | | N |
| ANISOU | 6723 | NH2 | ARG | P | 429 | 6560 | 5890 | 6437 | 487 | 439 | 197 | N |
| ATOM | 6724 | O | GLY | P | 430 | −12.500 | 0.641 | 9.683 | 1.00 | 33.44 | | O |
| ANISOU | 6724 | O | GLY | P | 430 | 4482 | 3601 | 4623 | 360 | 174 | 272 | O |
| ATOM | 6725 | N | GLY | P | 430 | −14.632 | −0.322 | 11.084 | 1.00 | 41.59 | | N |
| ANISOU | 6725 | N | GLY | P | 430 | 5560 | 4614 | 5628 | 392 | 296 | 246 | N |
| ATOM | 6726 | CA | GLY | P | 430 | −13.790 | −1.264 | 10.361 | 1.00 | 40.69 | | C |
| ANISOU | 6726 | CA | GLY | P | 430 | 5390 | 4501 | 5571 | 344 | 312 | 222 | C |
| ATOM | 6727 | C | GLY | P | 430 | −12.815 | −0.528 | 9.449 | 1.00 | 37.79 | | C |
| ANISOU | 6727 | C | GLY | P | 430 | 4989 | 4161 | 5207 | 327 | 244 | 234 | C |
| ATOM | 6728 | O | ILE | P | 431 | −13.217 | 0.553 | 6.316 | 1.00 | 33.85 | | O |
| ANISOU | 6728 | O | ILE | P | 431 | 4303 | 3864 | 4696 | 252 | 220 | 183 | O |
| ATOM | 6729 | N | ILE | P | 431 | −12.341 | −1.205 | 8.408 | 1.00 | 36.65 | | N |
| ANISOU | 6729 | N | ILE | P | 431 | 4768 | 4051 | 5106 | 277 | 263 | 203 | N |
| ATOM | 6730 | CA | ILE | P | 431 | −11.431 | −0.596 | 7.441 | 1.00 | 31.75 | | C |
| ANISOU | 6730 | CA | ILE | P | 431 | 4108 | 3463 | 4494 | 256 | 204 | 210 | C |
| ATOM | 6731 | C | ILE | P | 431 | −12.075 | 0.617 | 6.767 | 1.00 | 31.38 | | C |
| ANISOU | 6731 | C | ILE | P | 431 | 4033 | 3489 | 4399 | 264 | 172 | 213 | C |
| ATOM | 6732 | CB | ILE | P | 431 | −10.982 | −1.638 | 6.397 | 1.00 | 32.82 | | C |
| ANISOU | 6732 | CB | ILE | P | 431 | 4158 | 3629 | 4682 | 197 | 243 | 170 | C |
| ATOM | 6733 | CG1 | ILE | P | 431 | −10.183 | −2.745 | 7.083 | 1.00 | 34.89 | | C |
| ANISOU | 6733 | CG1 | ILE | P | 431 | 4452 | 3813 | 4992 | 191 | 263 | 172 | C |
| ATOM | 6734 | CG2 | ILE | P | 431 | −10.131 | −1.010 | 5.305 | 1.00 | 30.23 | | C |
| ANISOU | 6734 | CG2 | ILE | P | 431 | 3781 | 3343 | 4360 | 173 | 186 | 174 | C |
| ATOM | 6735 | CD1 | ILE | P | 431 | −8.873 | −2.256 | 7.701 | 1.00 | 35.00 | | C |
| ANISOU | 6735 | CD1 | ILE | P | 431 | 4532 | 3755 | 5013 | 216 | 188 | 220 | C |
| ATOM | 6736 | O | ILE | P | 432 | −11.819 | 4.214 | 4.076 | 1.00 | 32.03 | | O |
| ANISOU | 6736 | O | ILE | P | 432 | 4015 | 3759 | 4396 | 253 | −4 | 243 | O |
| ATOM | 6737 | N | ILE | P | 432 | −11.352 | 1.736 | 6.711 | 1.00 | 32.07 | | N |
| ANISOU | 6737 | N | ILE | P | 432 | 4149 | 3570 | 4466 | 283 | 90 | 251 | N |
| ATOM | 6738 | CA | ILE | P | 432 | −11.905 | 2.955 | 6.116 | 1.00 | 34.42 | | C |
| ANISOU | 6738 | CA | ILE | P | 432 | 4426 | 3934 | 4719 | 293 | 53 | 257 | C |
| ATOM | 6739 | C | ILE | P | 432 | −11.284 | 3.355 | 4.773 | 1.00 | 30.32 | | C |
| ANISOU | 6739 | C | ILE | P | 432 | 3834 | 3476 | 4211 | 255 | 16 | 247 | C |
| ATOM | 6740 | CB | ILE | P | 432 | −11.919 | 4.150 | 7.094 | 1.00 | 37.38 | | C |
| ANISOU | 6740 | CB | ILE | P | 432 | 4889 | 4268 | 5045 | 352 | −10 | 306 | C |
| ATOM | 6741 | CG1 | ILE | P | 432 | −10.559 | 4.332 | 7.760 | 1.00 | 40.47 | | C |
| ANISOU | 6741 | CG1 | ILE | P | 432 | 5345 | 4580 | 5453 | 372 | −74 | 349 | C |
| ATOM | 6742 | CG2 | ILE | P | 432 | −13.019 | 3.969 | 8.144 | 1.00 | 37.48 | | C |
| ANISOU | 6742 | CG2 | ILE | P | 432 | 4955 | 4256 | 5031 | 387 | 36 | 306 | C |
| ATOM | 6743 | CD1 | ILE | P | 432 | −10.449 | 5.644 | 8.535 | 1.00 | 44.51 | | C |
| ANISOU | 6743 | CD1 | ILE | P | 432 | 5936 | 5058 | 5916 | 426 | −148 | 399 | C |
| ATOM | 6744 | O | LYS | P | 433 | −8.171 | 0.912 | 3.440 | 1.00 | 32.29 | | O |
| ANISOU | 6744 | O | LYS | P | 433 | 3981 | 3672 | 4616 | 147 | 29 | 212 | O |
| ATOM | 6745 | N | LYS | P | 433 | −10.167 | 2.730 | 4.420 | 1.00 | 30.10 | | N |
| ANISOU | 6745 | N | LYS | P | 433 | 3782 | 3424 | 4229 | 226 | 7 | 244 | N |
| ATOM | 6746 | CA | LYS | P | 433 | −9.620 | 2.800 | 3.062 | 1.00 | 33.41 | | C |
| ANISOU | 6746 | CA | LYS | P | 433 | 4120 | 3905 | 4670 | 181 | −10 | 224 | C |
| ATOM | 6747 | C | LYS | P | 433 | −8.993 | 1.454 | 2.691 | 1.00 | 33.94 | | C |
| ANISOU | 6747 | C | LYS | P | 433 | 4143 | 3957 | 4797 | 137 | 36 | 194 | C |
| ATOM | 6748 | CB | LYS | P | 433 | −8.572 | 3.917 | 2.925 | 1.00 | 32.29 | | C |
| ANISOU | 6748 | CB | LYS | P | 433 | 4003 | 3751 | 4514 | 196 | −105 | 267 | C |
| ATOM | 6749 | CG | LYS | P | 433 | −9.060 | 5.302 | 3.329 | 1.00 | 29.89 | | C |
| ANISOU | 6749 | CG | LYS | P | 433 | 3748 | 3457 | 4153 | 241 | −159 | 301 | C |
| ATOM | 6750 | CD | LYS | P | 433 | −7.969 | 6.352 | 3.086 | 1.00 | 33.80 | | C |
| ANISOU | 6750 | CD | LYS | P | 433 | 4260 | 3944 | 4639 | 251 | −253 | 342 | C |
| ATOM | 6751 | CE | LYS | P | 433 | −8.434 | 7.761 | 3.476 | 1.00 | 31.89 | | C |
| ANISOU | 6751 | CE | LYS | P | 433 | 4067 | 3711 | 4339 | 296 | −310 | 376 | C |
| ATOM | 6752 | NZ | LYS | P | 433 | −7.389 | 8.761 | 3.068 | 1.00 | 34.77 | | N |
| ANISOU | 6752 | NZ | LYS | P | 433 | 4436 | 4078 | 4697 | 300 | −400 | 412 | N |
| ATOM | 6753 | O | THR | P | 434 | −7.088 | 0.909 | 0.074 | 1.00 | 30.52 | | O |
| ANISOU | 6753 | O | THR | P | 434 | 3548 | 3608 | 4441 | 34 | 6 | 154 | O |
| ATOM | 6754 | N | THR | P | 434 | −9.396 | 0.919 | 1.541 | 1.00 | 35.59 | | N |
| ANISOU | 6754 | N | THR | P | 434 | 4261 | 4237 | 5023 | 90 | 83 | 148 | N |
| ATOM | 6755 | CA | THR | P | 434 | −8.888 | −0.360 | 1.047 | 1.00 | 35.52 | | C |
| ANISOU | 6755 | CA | THR | P | 434 | 4201 | 4225 | 5071 | 45 | 130 | 114 | C |
| ATOM | 6756 | C | THR | P | 434 | −7.729 | −0.147 | 0.049 | 1.00 | 31.98 | | C |
| ANISOU | 6756 | C | THR | P | 434 | 3704 | 3800 | 4647 | 13 | 82 | 116 | C |
| ATOM | 6757 | CB | THR | P | 434 | −10.029 | −1.234 | 0.462 | 1.00 | 32.20 | | C |
| ANISOU | 6757 | CB | THR | P | 434 | 3712 | 3862 | 4659 | 12 | 217 | 60 | C |
| ATOM | 6758 | OG1 | THR | P | 434 | −10.738 | −0.492 | −0.539 | 1.00 | 32.65 | | O |
| ANISOU | 6758 | OG1 | THR | P | 434 | 3712 | 4008 | 4686 | −3 | 211 | 44 | O |
| ATOM | 6759 | CG2 | THR | P | 434 | −11.021 | −1.639 | 1.567 | 1.00 | 32.06 | | C |
| ANISOU | 6759 | CG2 | THR | P | 434 | 3748 | 3808 | 4627 | 43 | 267 | 59 | C |
| ATOM | 6760 | N | PHE | P | 435 | −7.440 | −1.137 | −0.802 | 1.00 | 31.73 | | N |
| ANISOU | 6760 | N | PHE | P | 435 | 3600 | 3795 | 4659 | −36 | 125 | 77 | N |
| ATOM | 6761 | CA | PHE | P | 435 | −6.234 | −1.085 | −1.658 | 1.00 | 31.87 | | C |
| ANISOU | 6761 | CA | PHE | P | 435 | 3576 | 3826 | 4707 | −66 | 84 | 79 | C |
| ATOM | 6762 | C | PHE | P | 435 | −6.339 | −0.069 | −2.801 | 1.00 | 29.79 | | C |
| ANISOU | 6762 | C | PHE | P | 435 | 3258 | 3646 | 4417 | −82 | 44 | 77 | C |
| ATOM | 6763 | O | PHE | P | 435 | −7.419 | 0.179 | −3.308 | 1.00 | 30.98 | | O |
| ANISOU | 6763 | O | PHE | P | 435 | 3369 | 3862 | 4540 | −91 | 73 | 54 | O |
| ATOM | 6764 | CB | PHE | P | 435 | −5.931 | −2.446 | −2.304 | 1.00 | 30.00 | | C |
| ANISOU | 6764 | CB | PHE | P | 435 | 3274 | 3599 | 4525 | −116 | 144 | 35 | C |
| ATOM | 6765 | CG | PHE | P | 435 | −5.662 | −3.565 | −1.320 | 1.00 | 31.76 | | C |
| ANISOU | 6765 | CG | PHE | P | 435 | 3540 | 3741 | 4784 | −108 | 183 | 33 | C |
| ATOM | 6766 | CD1 | PHE | P | 435 | −6.441 | −4.717 | −1.337 | 1.00 | 32.77 | | C |
| ANISOU | 6766 | CD1 | PHE | P | 435 | 3637 | 3880 | 4934 | −130 | 267 | −9 | C |
| ATOM | 6767 | CD2 | PHE | P | 435 | −4.621 | −3.473 | −0.406 | 1.00 | 29.15 | | C |
| ANISOU | 6767 | CD2 | PHE | P | 435 | 3282 | 3327 | 4468 | −81 | 136 | 75 | C |
| ATOM | 6768 | CE1 | PHE | P | 435 | −6.193 | −5.770 | −0.448 | 1.00 | 35.62 | | C |
| ANISOU | 6768 | CE1 | PHE | P | 435 | 4037 | 4167 | 5329 | −124 | 303 | −11 | C |
| ATOM | 6769 | CE2 | PHE | P | 435 | −4.360 | −4.515 | 0.490 | 1.00 | 29.66 | | C |
| ANISOU | 6769 | CE2 | PHE | P | 435 | 3386 | 3317 | 4566 | −75 | 172 | 73 | C |
| ATOM | 6770 | CZ | PHE | P | 435 | −5.153 | −5.665 | 0.468 | 1.00 | 32.59 | | C |
| ANISOU | 6770 | CZ | PHE | P | 435 | 3725 | 3700 | 4958 | −97 | 256 | 30 | C |
| ATOM | 6771 | O | SER | P | 436 | −2.853 | 0.265 | −4.625 | 1.00 | 35.91 | | O |
| ANISOU | 6771 | O | SER | P | 436 | 3954 | 4425 | 5266 | −146 | −87 | 105 | O |
| ATOM | 6772 | N | SER | P | 436 | −5.195 | 0.475 | −3.216 | 1.00 | 28.46 | | N |
| ANISOU | 6772 | N | SER | P | 436 | 3084 | 3474 | 4257 | −89 | −22 | 102 | N |
| ATOM | 6773 | CA | SER | P | 436 | −5.063 | 1.195 | −4.487 | 1.00 | 33.17 | | C |
| ANISOU | 6773 | CA | SER | P | 436 | 3613 | 4149 | 4841 | −116 | −56 | 94 | C |
| ATOM | 6774 | C | SER | P | 436 | −3.845 | 0.674 | −5.250 | 1.00 | 35.26 | | C |
| ANISOU | 6774 | C | SER | P | 436 | 3829 | 4414 | 5152 | −155 | −68 | 84 | C |
| ATOM | 6775 | CB | SER | P | 436 | −4.908 | 2.702 | −4.256 | 1.00 | 31.56 | | C |
| ANISOU | 6775 | CB | SER | P | 436 | 3455 | 3947 | 4591 | −79 | −139 | 141 | C |
| ATOM | 6776 | OG | SER | P | 436 | −6.064 | 3.244 | −3.639 | 1.00 | 36.23 | | O |
| ANISOU | 6776 | OG | SER | P | 436 | 4084 | 4544 | 5136 | −44 | −129 | 148 | O |
| END | | | | | | | | | | | | |

In the complex between the 15-residue F peptide and 101F, only the amino acids KNRGIIKTFS (SEQ ID NO:4) are modeled in the crystal structure as the remaining residues are disordered. As such, the coordinates disclosed in PDB acc code 3O41 only include those 10 amino acids of the 15-residue peptide.

### Example 3

### Production and Characterization of Scaffold-Based Immunogens

This Example describes the production and testing of scaffold-based immunogens designed using the atomic coordinates in PDB acc code 3IXT, i.e., the atomic coordinates of the complex between the 24-residue F peptide comprising the motavizumab binding domain and motavizumab.

Several scaffolds were designed to present the motavizumab epitope, or binding domain, using the superposition method (also referred to as side chain grafting) and the multi-segment side chain grafting method (also referred to as double superpositioning). Multi-segment side chain grafting is an extension of the superposition method described in WO 2008/025015 A2. Multi-segment side chain grafting is intended for transplantation of certain complex epitopes to scaffold proteins, in which the epitope contains two or more backbone segments in a fixed orientation relative to each other; e.g., the motavizumab epitope is composed of two helices. Although the description herein focuses on two backbone segments, it is to be appreciated that the algorithm is generalizable to any number of segments. The method works very similarly to the original superposition method, except that scaffolds are scanned for structural similarity to each of the two epitope segments individually, and whenever a match is found to one of the segments, that scaffold is searched a second time for structural similarity to the other epitope segment, with the rigid body position of the second epitope segment relative to the scaffold pre-determined by the superposition of the first segment to the scaffold. “Double” matches are identified if (a) one epitope segment matches a scaffold with backbone rmsd/nsup<threshold 1 and the other segment matches with backbone rmsd/nsup<threshold 2, where threshold 1 is typically 0.15 and threshold 2 is typically 0.2, or (b) if both segments are superimposed simultaneously onto the scaffold and the backbone rmsd/nsup is <threshold 3, where threshold 3 is typically 0.2. As used herein, backbone rmsd refers to the root-mean square deviation of a structural alignment computed over the backbone atoms N, CA, C, O; and backbone rmsd/nsup refers to backbone rmsd divided by the number of aligned residues.

An automated search of monomeric and non-monomeric proteins without co-factors within the PDB (RCSB Protein Data Bank, Brookhaven, N.Y.) was conducted in March 2009 to identify candidate scaffolds having similar three-dimensional structures to the three-dimensional structure defined by the atomic coordinates specified in PDB acc code 3IXT. Nearly all of the structural matches contained significant backbone clashes with the motavizumab antibody (multiple atomic overlaps). Several hundred structural matches were screened by eye to identify structures that could be trimmed to eliminate backbone clash. Ten variants of 3 different scaffold proteins (1lp1b (Staphylococcus aureus Protein A), 1s2xa (Helicobacter pylori CagZ protein) and 2eiaa (equine infectious anemia virus) were chosen either because no trimming was necessary (1lp1b, 2eiaa) or the necessary trimming was restricted to a terminus and could be done easily (1s2xa). It is to be appreciated that this method can also identify scaffolds requiring more advanced trimming by flexible backbone protein design.

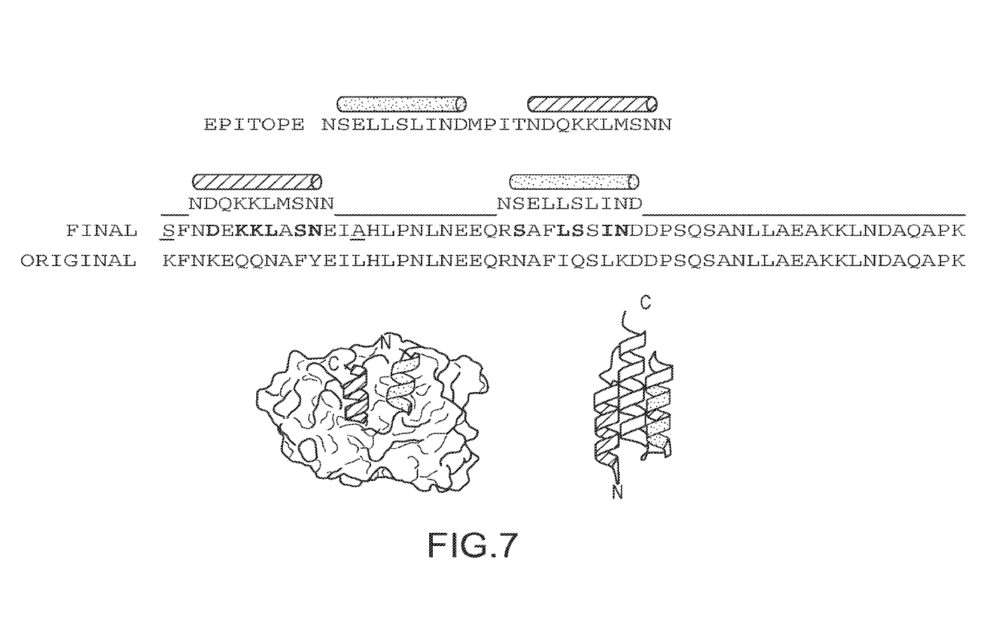

Residues required for elicitation of a humoral immune response against RSV were then implanted into the scaffolds and epitope conformation was stabilized by genetic engineering using a method similar to that described in WO 2008/025015 A2. For example, FIG. 7 shows a final scaffold comparison for immunogen 1lp1b_{—}003. Amino acids in bold are motavizumab contact residues. Amino acids that are underlined were substituted to stabilize the structure.

The amino acid sequences of ten scaffold-based immunogens are:

11p1b_001
(SEQ ID NO: 18)
VDNSFNDEKKLASNEIAHLPNLNEEQRSAFLSSINDDPSQSANLLAEA
KKLNDAQAPK
11p1b_002
(SEQ ID NO: 21)
VDNSFNDEKKLASNEIQHLPNLNEEQRSAFISSLNDDPSQSANLLAEA
KKLNDAQAPK
11p1b_003
(SEQ ID NO: 24)
SFNDEKKLASNEIAHLPNLNEEQRSAFLSSINDDPSQSANLLAEAKKL
NDAQAPK
11p1b_004
(SEQ ID NO: 149)
SFNDEKKLASNEIQHLPNLNEEQRSAFISSLNDDPSQSANLLAEAKKL
NDAQAPK
1s2xa_001
(SEQ ID NO: 152)
GSPNSRVDELGFNEAERQKILDSNSSLMRNANEVRDKFIQNYATSLKD
SNDPQDFLRRVQELRINMQKNFISFDAYYNYLNNLVLASANRCSQELS
FANDTIKNNDTKKLFSNEIADNFNNFTADEVARISDLVASYLPREYLP
PFIDGNMMGVAFQILGIDDFGKKLNEIVQDIGTKY
1s2xa_002
(SEQ ID NO: 155)
GSPNSRVDELGFNEAERQKILDSNSSLMRNANEVRDKFIQNYATSLKD
SNDPQDFLRRVQELRINMQKNFISFDAYYNYLNNLVLASANRASQELS
FINDTIKNNDTKKLFSNEAADNFNNFTADEVARISDLVASYLPREYLP
PFIDGNMMGVAFQILGIDDFGKKLNEIVQDIGTKY
1s2xa_003
(SEQ ID NO: 158)
GSPNSRVDELGFNEAERQKILDSNSSLMRNANEVRDKFIQNYATSLKD
SNDPQDFLRRVQELRINMQKNFISFDAYYNYLNNLVLASANRASQELS
FANDTIKNNDTKKLFSNEIADNFNNFTADEVARISDLVASYLPREYLP
PFIDGNMMGVAFQILGIDDFGKKLNEIVQDIGTK
1s2xa_004
(SEQ ID NO: 164)
GSPNSRVDELGFNEAERQKILDSNSSLMRNANEVRDKFIQNYATSLKD
SNDPQDFLRRVQELRINMQKNFISFDAYYNYLNNLVLASANRASQELS
FINDTIKNNDTKKLFSNEAADNFNNFTADEVARISDLVASYLPREYLP
PFIDGNMMGVAFQILGIDDFGKKLNEIVQDIGTK
2eiaa_001
(SEQ ID NO: 167)
APRGYTTWVNTIQTNGLLNEASQNLFGILSVDATSEEMNAFLDVVPGQ
AGQKQILLDAIDKIADDWDNRHPLPNAPLVAPPQGPIPMTARFIRGLG
VPRERQMEPAFDQFRQTYRQWIIEAMSEGIKVMIGKPKAQNIRQGAKE
PYPEFVSRLLSQINDEGHPNDIKKLRSNTLTIQNANEECRNAMRHLRP
SDTGAEKMYACRDIG
2eiaa_002
(SEQ ID NO: 170)
GKPKAQNIRQGAKEPYPEFVSRLLSQINDEGHPNDIKKLRSNTLTIQN
ANEECRNAMRHLRPSDTGAEKMYACRDIG

These immunogens have been further tailored to include amino (_N) or carboxyl (_C) tags or motifs to aid in purification of immunogens of the embodiments. Examples include of production of the following immunogens:

1sx2a_001_N_His
(SEQ ID NO: 177)
HHHHHHGSPNSRVDELGFNEAERQKILDSNSSLMRNANEVRDKFIQN
YATSLKDSNDPQDFLRRVQELRINMQKNFISFDAYYNYLNNLVLASA
NRCSQELSFANDTIKNNDTKKLFSNEIADNFNNFTADEVARISDLVA
SYLPREYLPPFIDGNMMGVAFQILGIDDFGKKLNEIVQDIGTKY*
1sx2a_002_N_His
(SEQ ID NO: 178)
HHHHHHGSPNSRVDELGFNEAERQKILDSNSSLMRNANEVRDKFIQN
YATSLKDSNDPQDFLRRVQELRINMQKNFISFDAYYNYLNNLVLASA
NRASQELSFINDTIKNNDTKKLFSNEAADNFNNFTADEVARISDLVA
SYLPREYLPPFIDGNMMGVAFQILGIDDFGKKLNEIVQDIGTKY*
1sx2a_003_N_His
(SEQ ID NO: 179)
HHHHHHGSPNSRVDELGFNEAERQKILDSNSSLMRNANEVRDKFIQN
YATSLKDSNDPQDFLRRVQELRINMQKNFISFDAYYNYLNNLVLASA
NRASQELSFANDTIKNNDTKKLFSNEIADNFNNFTADEVARISDLVA
SYLPREYLPPFIDGNMMGVAFQILGIDDFGKKLNEIVQDIGTK*
1sx2a_004_N_His
(SEQ ID NO: 180)
HHHHHHGSPNSRVDELGFNEAERQKILDSNSSLMRNANEVRDKFIQN
YATSLKDSNDPQDFLRRVQELRINMQKNFISFDAYYNYLNNLVLASA
NRASQELSFINDTIKNNDTKKLFSNEAADNFNNFTADEVARISDLVA
SYLPREYLPPFIDGNMMGVAFQILGIDDFGKKLNEIVQDIGTK*
2eiaa_002_N_His
(SEQ ID NO: 181)
HHHHHHGKPKAQNIRQGAKEPYPEFVSRLLSQINDEGHPNDIKKLRS
NTLTIQNANEECRNAMRHLRPSDTGAEKMYACRDIG*

Each of the four 1lp1b-based immunogens, namely 1lp1b_{—}001, 1lp1b_{—}002, 1lp1b_{—}003 and 1lp1b_{—}004 was expressed with a HRV3C site, PADRE, Caspase3 site, 6×His tag and StrepTagII in 293F mammalian cells (Invitrogen) transformed with paH (also known as p(alpha)H) vector comprising a nucleic acid sequence encoding the respective immunogen. The paH vector is a modified version of the pHLSec vector (Aricescu A R et al, ibid) that includes changes to the multi-cloning site (MCS) and removal of certain restriction enzyme sites. The resultant immunogens were purified by nickel IMAC and STREP-TACTIN® chromatography followed by gel filtration. His and Strep tags were cleaved by pro-caspase. As an example, the following sequence is 1lp1b_{—}001 with a HRV3C site (LEVLFQGP (SEQ ID NO:182)), PADRE (AKFVAAWTLKAAA (SEQ ID NO:183)), caspase3 site (DEVD (SEQ ID NO:184), 6×His tag (HHHHHH (SEQ ID NO:185)) and StrepTagII (WSHPQFEK (SEQ ID NO:186)) (each underlined) at its carboxyl terminus:

VDNSFNDEKKLASNEIAHLPNLNEEQRSAFLSSINDDPSQSANLLAE
AKKLNDAQAPKLEVLFQGPGAKFVAAWTLKAAAGDEVDGSHHHHHHS
AWSHPQFEK,
also denoted herein as_SEQ ID NO: 187.

Each of three of the four 1s2xa-based immunogens, namely 1s2xa_{—}001, 1s2xa_{—}002, and 1s2xa_{—}003, was expressed as a maltose binding protein fusion in BL21(DE3) bacteria transformed with MBP-HTSHP vector comprising a nucleic acid sequence encoding the respective immunogen. The MBP-HTSHP vector is a modified version of the pMal-c2x vector (New England Biolabs, Ipswich, Mass.) that includes a linker region with all the various tags and restriction sites. Fusion protein was recovered by nickel chromatography. The fusion protein was cleaved with pro-caspase 3 and subjected to nickel chromatography and S75 gel filtration. An anion exchange column can also be used as part of the procedure. As an example, the following sequence in bold is 1s2xa_{—}001 with maltose-binding protein, a factor Xa site, his-tag, TEV site, strep-tag, his-tag, HRV3C site and caspase 3 site (each underlined) at its amino terminus:

MKIEEGKLVIWINGDKGYNGLAEVGKKFEKDTGIKVTVEHPDKLEEK
FPQVAATGDGPDIIFWAHDRFGGYAQSGLLAEITPDKAFQDKLYPFT
WDAVRYNGKLIAYPIAVEALSLIYNKDLLPNPPKTWEEIPALDKELK
AKGKSALMFNLQEPYFTWPLIAADGGYAFKYENGKYDIKDVGVDNAG
AKAGLTFLVDLIKNKHMNADTDYSIAEAAFNKGETAMTINGPWAWSN
IDTSKVNYGVTVLPTFKGQPSKPFVGVLSAGINAASPNKELAKEFLE
NYLLTDEGLEAVNKDKPLGAVALKSYEEELAKDPRIAATMENAQKGE
IMPNIPQMSAFWYAVRTAVINAASGRQTVDEALKDAQTNSSSNNNNN
NNNNNLGIEGRISGHHHHHHHHDYDIPSSENLYFQGSASWSHPQFEK
SGHHHHHHHHDYDIPSSLEVLFQGPGSDEVDGSPNSRVDELGFNEAE
RQKILDSNSSLMRNANEVRDKFIQNYATSLKDSNDPQDFLRRVQELR
INMQKNFISFDAYYNYLNNLVLASANRCSQELSFANDTIKNNDTKKL
FSNEIADNFNNFTADEVARISDLVASYLPREYLPPFIDGNMMGVAFQ
ILGIDDFGKKLNEIVQDIGTKY,
also denoted herein as SEQ ID NO: 151.

Each immunogen of the present invention can be produced in the manner described herein.

Scaffold-based immunogens 1lp1b_{—}001, 1lp1b_{—}002, 1lp1b_{—}003, 1lp1b_{—}004, 1s2xa_{—}001, 1s2xa_{—}002 and 1s2xa_{—}003 were submitted to surface plasmon resonance binding analysis. Experiments were carried out on a Biacore 3000 instrument (GE Healthcare). Motavizumab fragment of antigen binding (Fab) was covalently coupled to a CM5 chip, and a blank surface with no antigen was created under identical coupling conditions for use as a reference. Scaffolds were serially diluted 2-fold, starting at 10 mM, into 10 mM HEPES pH 7.4, 150 mM NaCl, 3 mM EDTA and 0.005% polysorbate 20 and injected over the immobilized motavizumab Fab and reference cell at 40 ml/min. The surface was regenerated with 10 mM glycine pH 3 at a flow rate of 40 ml/min. The data were processed with SCRUBBER-2 and double referenced by subtraction of the blank surface and a blank injection (no analyte).

Results are shown in Table 5, which indicates the Kd in nanomolar (nM) concentrations for each of the tested scaffold-based immunogens.

TABLE 5
Affinity of scaffold-based immunogens for motavizumab

| Scaffold | Motavizumab affinity (K_{d} in nM) |
|---|---|
| 1lp1b_003 | 66 |
| 1lp1b_001 | 181 |
| 1lp1b_004 | 296 |
| 1lp1b_002 | 700 |
| 1s2xa_003 | 3005 |
| 1s2xa_002 | 3638 |
| 1s2xa_001 | 4755 |

Immunogen 1lp1b_{—}003 exhibited the best affinity for motavizumab in this assay. This immunogen was also tested for binding to an irrelevant IgG and irrelevant Fab. No significant binding above baseline was detected.

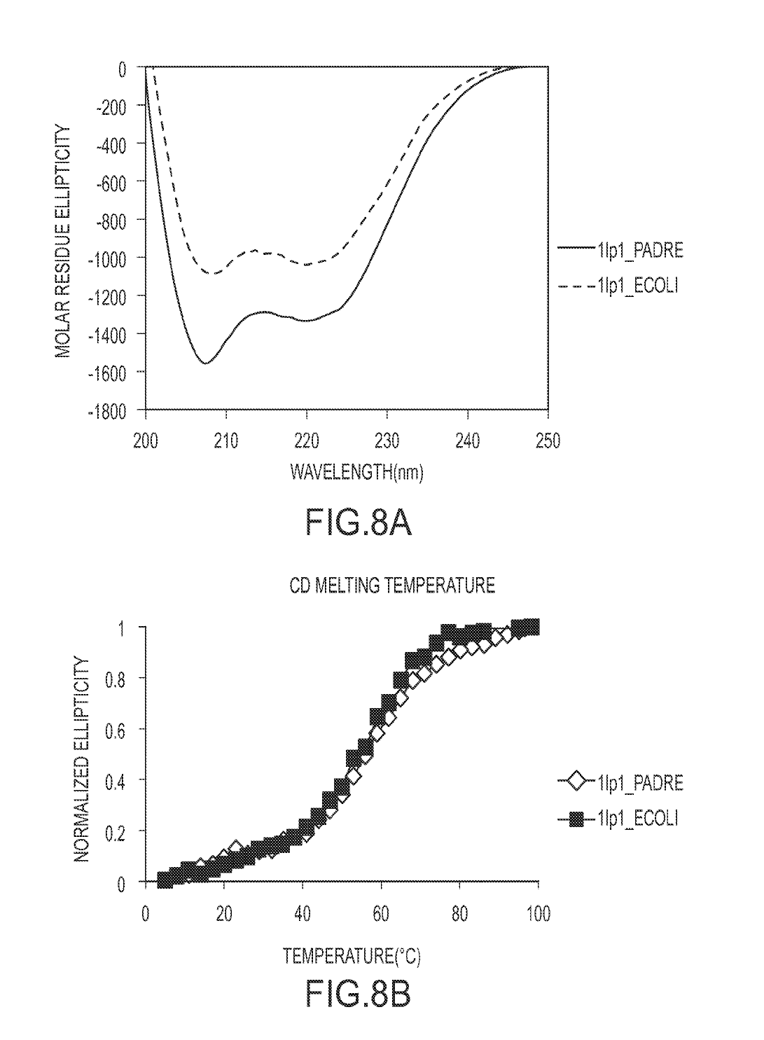

Immunogen 1lp1b_{—}003 proteins, produced in both mammalian cells and bacteria as described above, were also tested for their three-dimensional structure. The data in FIG. 8 demonstrate that both mammalian- and bacterial-produced 1lp1b_{—}003 proteins are alpha helical in solution and have a melting temperature of about 60° C., which confirms that the proteins are folded in solution.

### Example 4

### Production of Resurfaced Scaffold-Based Immunogens

This example describes the production of scaffold-based immunogens that have been resurfaced to neutralize deleterious immunodominant epitopes.

Resurfaced variants of the 1lp1b_{—}003 scaffold were designed using protocols similar to those described in WO 2009/100376 A2. This scaffold has the highest binding affinity of the initial designs. In these resurfaced variants, a significant fraction of the non-epitope surface area on the scaffold was mutated to generate antigenic surfaces distinct from the original design. In a few cases the mutations were designed to introduce N-linked glycosylation sites, but in most cases the mutations did not. These variants are useful in heterologous prime-boost immunizations using a non-resurfaced immunogen as the prime and one or more of the resurfaced variants as a boost, with the aim of focusing the antibody response onto the antibody binding domain, or epitope, which is intended to be the only antigenic surface conserved between the non-resurfaced and resurfaced immunogens.

The sequences for several resurfaced 1lp1b immunogens are below. The first two immunogens include substitutions resulting in glycan-masked immunogens; the remaining are non-glycan resurfaced immunogens.

mota_11p1b.m1.cl.d1_glyc1
(SEQ ID NO: 55)
SFNDEKKLASNEIAHLPNLNETQRSAFLSSINDDPSQSANLLANAT
KLNDAQAP
mota_11p1b.m1.cl.d1_glyc2
(SEQ ID NO: 56)
SFNDEKKLASNEIAHLPNLNETQRSAFLSSINDDPNQSANLLANAT
KLNDAQAP
mota_11p1b.m1.c1.d1_des1_1
(SEQ ID NO: 66)
SFNDEKKLASNRIANLPNLNEEQRSAFLSKINDDPSQSANLLEEAL
KLNDAQAQK
mota_11p1b.m1.c1.d1_des1_2
(SEQ ID NO: 67)
SFNDDKKLASNRIANLPNLNEEQRSAFLSKINDDPSQSRNLLEEAL
KLNDAQAQK
mota_11p1b.m1.c1.d1_des1_3
(SEQ ID NO: 68)
SFNDKKKLASNRIANLPNLNEEQRSAFLSKINDDPSKSEELLEKAL
KLNDAQAQK
mota_11p1b.m1.c1.d1_des1_5
(SEQ ID NO: 69)
SFNDKKKLASNEIANLPNLNEEQRSAFLSKINDDPSKSEELLEEAL
KLNDAQADK
mota_11p1b.m1.c1.d1_des1_6
(SEQ ID NO: 70)
SFNDKKKLASNRIAKLPNLNEKQRSAFLSKINDDPSKSEELLKKAL
KLNKAQAKK
mota_11p1b.m1.c1.d1_des1_7
(SEQ ID NO: 71)
SFNDEKKLASNRIANLPNLNQEQRSAFLSKINDDPSQSANLLEEAL
KLNDNQAQK
mota_11p1b.m1.c1.d1_des1_8
(SEQ ID NO: 72)
SFNDDKKLASNRIANLPNLNQEQRSAFLSKINDDPSQSRNLLEEAL
KLNDNQAQK
mota_11p1b.m1.c1.d1_des1_9
(SEQ ID NO: 73)
SFNDKKKLASNRIANLPNLNQEQRSAFLSKINDDPSKSEELLEKAL
KLNDNQAQK
mota_11p1b.m1.c1.d1_des1_10
(SEQ ID NO: 74)
SFNDKKKLASNEIANLPNLNQEQRSAFLSKINDDPSKSEELLEEAL
KLNDNQADK
mota_11p1b.m1.c1.d1_des1_11
(SEQ ID NO: 75)
SFNDKKKLASNRIAKLPNLNEKQRSAFLSNINDDPSKSEELLEKAL
KLNQAQAQK

Production of these resurfaced immunogens can occur using methods similar to those described in Example 3.

### Example 5

### Production of Trimeric F Immunogens

The production of RSV Fₒ Fd immunogen was described in Example 1. Additional soluble trimeric F immunogens, stabilized in the pre-fusion conformation, namely RSV F Fd and RSV F₀ Fd GAG, have been produced in a similar manner. The amino acid sequence of each of these immunogens, with the T4 fibritin trimerization domain (also referred to as Foldon, or Fd) underlined, is as follows:

RSV F₀ Fd
(SEQ ID NO: 174)
MELLILKANAITTILTAVTFCFASGQNITEEFYQSTCSAVSKGYLSA
LRTGWYTSVITIELSNIKENKCNGTDAKVKLIKQELDKYKNAVTELQ
LLMQSTPATNNQARSELPRFMNYTLNNAKKTNVTLSKKRKSSFLGFL
LGVGSAIASGVAVSKVLHLEGEVNKIKSALLSTNKAVVSLSNGVSVL
TSKVLDLKNYIDKQLLPIVNKQSCSISNIETVIEFQQKNNRLLEITR
EFSVNAGVTTPVSTYMLTNSELLSLINDMPITNDQKKLMSNNVQIVR
QQSYSIMSIIKEEVLAYVVQLPLYGVIDTPCWKLHTSPLCTTNTKEG
SNICLTRTDRGWYCDNAGSVSFFPQAETCKVQSNRVFCDTMNSLTLP
SEVNLCNVDIFNPKYDCKIMTSKTDVSSSVITSLGAIVSCYGKTKCT
ASNKNRGIIKTFSNGCDYVSNKGVDTVSVGNTLYYVNKQEGKSLYVK
GEPIINFYDPLVFPSDEFDASISQVNEKINQSLAFIRKSDELLSAIG
GYIPEAPRDGQAYVRKDGEWVLLSTFLGGIEGRHHHHHH-
RSV F Fd
(SEQ ID NO: 175)
MELLILKANAITTILTAVTFCFASGQNITEEFYQSTCSAVSKGYLSA
LRTGWYTSVITIELSNIKENKCNGTDAKVKLIKQELDKYKNAVTELQ
LLMQSTPATNNRARRELPRFMNYTLNNAKKTNVTLSKKRKRRFLGFL
LGVGSAIASGVAVSKVLHLEGEVNKIKSALLSTNKAVVSLSNGVSVL
TSKVLDLKNYIDKQLLPIVNKQSCSISNIETVIEFQQKNNRLLEITR
EFSVNAGVTTPVSTYMLTNSELLSLINDMPITNDQKKLMSNNVQIVR
QQSYSIMSIIKEEVLAYVVQLPLYGVIDTPCWKLHTSPLCTTNTKEG
SNICLTRTDRGWYCDNAGSVSFFPQAETCKVQSNRVFCDTMNSLTLP
SEVNLCNVDIFNPKYDCKIMTSKTDVSSSVITSLGAIVSCYGKTKCT
ASNKNRGIIKTFSNGCDYVSNKGVDTVSVGNTLYYVNKQEGKSLYVK
GEPIINFYDPLVFPSDEFDASISQVNEKINQSLAFIRKSDELLSAIG
GYIPEAPRDGQAYVRKDGEWVLLSTFLGGLVPRGSHHHHHHSAWSHP
QFEK--
RSV F₀ Fd GAG
(SEQ ID NO: 176)
GAGMELLILKANAITTILTAVTFCFASGQNITEEFYQSTCSAVSKGY
LSALRTGWYTSVITIELSNIKENKCNGTDAKVKLIKQELDKYKNAVT
ELQLLMQSTPATNNRARGAGKRRFLGFLLGVGSAIASGVAVSKVLHL
EGEVNKIKSALLSTNKAVVSLSNGVSVLTSKVLDLKNYIDKQLLPIV
NKQSCSISNIETVIEFQQKNNRLLEITREFSVNAGVTTPVSTYMLTN
SELLSLINDMPITNDQKKLMSNNVQIVRQQSYSIMSIIKEEVLAYVV
QLPLYGVIDTPCWKLHTSPLCTTNTKEGSNICLTRTDRGWYCDNAGS
VSFFPQAETCKVQSNRVFCDTMNSLTLPSEVNLCNVDIFNPKYDCKI
MTSKTDVSSSVITSLGAIVSCYGKTKCTASNKNRGIIKTFSNGCDYV
SNKGVDTVSVGNTLYYVNKQEGKSLYVKGEPIINFYDPLVFPSDEFD
ASISQVNEKINQSLAFIRKSDELLSAIGGYIPEAPRDGQAYVRKDGE
WVLLSTFLGGLVPRGSHHHHHHSAWSHPQFEK--

Gel filtration analysis of RSV Fₒ Fd immunogen indicates that this immunogen has a calculated molecular weight of about 240 kilodaltons (kD), indicative of a trimer (FIG. 2 e). Binding studies indicate that RSV Fₒ Fd immunogen binds motavizumab, SYNAGIS® and 101F with affinities similar to RSV F protein. RSV Fₒ Fd immunogen can bind motavizumab and 101F simultaneously.

### Example 6

### Design of Antibodies Having Higher Affinity Binding for the Motavizumab-Binding Domain

This Example describes a method to design antibodies having a higher binding affinity for the motavizumab-binding domain. Such antibodies can have utility as passive immunotherapy compositions.

Additional interactions were identified between the motavizumab antibody and its epitope using a modified Rosetta interface design protocol. The crystal structure of the complex between motavizumab and its binding domain was prepacked to remove clashes as calculated by Rosetta. The iterations of local refine docking between epitope and antibody were carried out to generate an ensemble of slightly different rigid body orientations, then the backrub algorithm was applied to both epitope and antibody to generate backbone conformational variation, and finally iterative design and minimization of sidechains in the interface was carried out. This protocol was carried out hundreds of thousands of times; 20,000 low-energy structures were generated, and the top 1% were selected based on calculated ddG. From those 200 structures, approximately 50 were selected based on the total calculated free energy. The top 50 structures were aligned and the mutations listed below were identified as ones that could enhance binding between the antibody and peptide. Several candidate mutations are listed at many of the positions. A DNA library encoding all of these mutations within a single-chain fragment variable (ScFv) construct for motavizumab will be built and screened using yeast surface display to identify the tightest binding clones.

The mutations identified that may increase binding affinity are as follows:

| Motavizumab heavy (H) chain residue | Encoded mutation |
|---|---|
| 32 | H or E |
| 35 | A |
| 52 | K, H, T, S or R |
| 53 | H or S |
| 54 | F or R |
| 56 | I, S, E or D |
| 58 | Y or W |
| 97 | D, H or R |
| 99 | D |
| 100 | Y, W or H |
| 100A | S or T |

| Motavizumab light (L) chain residue | Encoded mutation |
|---|---|
| 32 | F |
| 49 | H or R |
| 92 | K |
| 94 | H |
| 96 | H |

It was also recognized that additional contacts between the antibody and epitope could be made by increasing the length of the CDRH2 loop by 2 residues. The CDRH2 loop in the heavy chain of motavizumab spans amino acids H50 through H58. The above protocol was used for these simulations as well but it included an additional step in which the loop was explicitly rebuilt to increase the length prior to the iterations of docking, backrub and design. Following is the library of mutations for that lengthened loop. This library is merged with the library above and screened on yeast as described above.

Specifically, the 9-residue stretch between H50-H58 was removed and replaced with a variation of the 11-residue sequence below, were 1 is the first amino acid in that sequence:

| Position | Encoded mutation |
|---|---|
| 1 | E, S or M |
| 2 | I |
| 3 | H, R or F |
| 4 | S |
| 5 | G |
| 6 | G, H, K, L, N, Q, S, D, T or R |
| 7 | F, K, S, T, D or R |
| 8 | E, N or D |
| 9 | D, H, L, S, R or T |
| 10 | Y |
| 11 | Y, F or H. |

### Example 7

### Binding of Immunogens to Motavizumab

This Example demonstrates the ability of immunogens of the present disclosure to bind to motavizumab.

a. Expression protocol for 1lp1b-based immunogens: Mammalian codon-optimized genes encoding 1lp1b-based immunogens were synthesized by GeneArt with an N-terminal secretion signal (MGSLQPLATLYLLGMLVASVLA (SEQ ID NO:188)) and a C-terminal HRV3C cleavage site, PADRE epitope (AKFVAAWTLKAAA (SEQ ID NO:183)), Caspase-3 cleavage site, 6×His-tag and Strep-tag II. The genes were cloned into the mammalian expression vector paH. Proteins were expressed from the plasmids by transient transfection using the FREESTYLE™ 293 expression system (Invitrogen). 1lp1b proteins were purified from the media using Ni²⁺-NTA resin (Qiagen) and then STREP-TACTIN® resin (IBA, Goettingen, Germany) as per manufacturer's instructions, followed by passage over a 16/60 Superdex 75 column (GE Healthcare). For SPR, ITC and CD measurements, all tags were retained. For immunization experiments, Pro-caspase 3 was added to remove the 6×His-tag and Strep-tag II. The tags and protease were removed from cleaved 1lp1b by passage over Ni²⁺-NTA resin.

b. Expression protocol for 1s2xa-based immunogens: E. coli codon-optimized genes encoding 1s2xa-based immunogens were synthesized by GeneArt and cloned into a custom vector based on pMAL-c2X (New England Biolabs). The expression vectors were transformed into BL21(DE3) cells, and the cells were grown in Terrific Broth (Difco, Becton Dickinson, Franklin Lakes, N.J.) at 37° C. until OD₆₀₀=2.0. The temperature was then reduced to 22° C., and isopropyl f3-D-thiogalactoside (IPTG) was added to 1 mM. After overnight incubation at 22° C., the cells were harvested and lysed with BUGBUSTER™ Protein Extraction Reagent (Novagen, EMD Chemicals, Gibbstown, N.J.), and 1s2xa proteins were purified using Ni²⁺-NTA resin (Qiagen). Fusion tags were removed by incubation with Pro-caspase 3 and passage over Ni²⁺-NTA resin. 1s2xa proteins were further purified by passage over a 16/60 SUPERDEX™ 75 column (GE Healthcare), and anion exchange chromatography using a MonoQ column (GE Healthcare).

c. Production of ferritin-containing immunogens. The gene encoding 1lp1b_{—}003 fused to the N-terminus of the coding region of human ferritin was subcloned into a mammalian expression vector, such a pVRC8405 (Barourch D et al., 2005, J. Virol. 79, 8828-8834). Proteins were expressed from this plasmid by transient transfection in HEK293 GnTI^{-/-} cells. 1lp1b_{—}003 ferritin was initially purified from the media by anion exchange chromatography using a MonoQ HR 10/10 column (GE Healthcare). The eluted protein was then passed over a column consisting of SYNAGIS® IgG covalently coupled to Protein A agarose resin (Pierce). The column was washed with phosphate-buffered saline (PBS) and eluted with Actisep Elution Medium (Sterogene, Carlsbad, Calif.). The eluted 1lp1b_{—}003 ferritin was dialyzed against PBS, concentrated, and further purified by passage over a 16/70 SUPEROSE™6 column (GE Healthcare). Additional ferritin-containing immunogens, such as 1lp1b_{—}003_eumS, 1lp1b_{—}003_eumSP, 1lp1b_{—}003eumL, and 1lp1b_{—}003_eumLP, were produced in a similar manner.

Immunogens were produced as described herein and submitted to surface plasmon resonance binding analysis as described in Example 3. Some of the immunogens were also submitted to isothermal titration calorimetry (ITC). Table 8 indicates which immunogens were tested and the results obtained. K_{d} refers to the K_{d} of motavizumab for a tested immunogen by either Biacore or ITC measurement, respectively. dH and -TdS relate to measurements of enthalpy and entropy, respectively. Table 8 also indicates the cell type (i.e., HEK293 mammalian cells or E. coli bacteria) used to produce the respective immunogen. Table 8 also provides the name of the immunogen used in Table 9. A blank square indicates that the respective immunogen was not tested in that assay. “nd” means not detectable; i.e., below the limit of detection of the assay.

TABLE 8
Affinity of immunogens for motavizumab and ITC measurements

| | | Immunogen | | | | |
|---|---|---|---|---|---|---|
| | Name used in | production | Biacore | ITC | | |
| Name | Table 9 | system | K_{d} (nM) | dH | −TdS | K_{d} (nM) |
| 1lp1b_001 | | HEK293 | 181 | | | |
| 1lp1b_002 | | HEK293 | 700 | | | |
| 1lp1b_003 | 1lp1b | HEK293 | 66 | 1.1 | −10.5 | 126 |
| 1lp1b_003 | 1lp1b(Ecoli) | E. coli | | | | |
| 1lp1b_004 | | HEK293 | 296 | | | |
| 1lp1b_003_K272E | | HEK293 | nd | | | |
| 1l1pb_003_Glyc1 | 1lp1b_Glyc1 | HEK293 | 400 | | | |
| 1lp1b_003_Glyc2 | 1lp1b_Glyc2 | HEK293 | 812 | | | |
| 1l1pb_003_Surf1 | 1lp1b_Surf1 | HEK293 | 120 | | | |
| 1l1pb_003_Surf8 | 1lp1b_Surf8 | HEK293 | 117 | | | |
| 1s2xa_001 | | E. coli | 4755 | | | |
| 1s2xa_002 | | E. coli | 3638 | | | |
| 1s2xa_003 | 1s2xa or Cag-Z | E. coli | 3005 | −19.2 | 9.9 | 147 |
| 1s2xa_004 | | E. coli | 3950 | −19.6 | 10.7 | 275 |
| 2eiaa_001 | | HEK293 | nd | | | |
| 1lp1b_003_K46A | K46A | HEK293 | 148 | −5.3 | −5.1 | 21 |
| 1lp1b_003_Q52A | Q52A | HEK293 | 233 | −1.8 | −8.8 | 16 |
| 1lp1b_003_I13L_F27A | I13L F27A | HEK293 | 541 | −19.4 | 10.1 | 159 |
| 1lp1b-003_L41I_L42A | L41I L42A | HEK293 | 247 | −1.3 | −9.0 | 24 |
| 1lp1b_003_L41I_L42V | L41I L42V | HEK293 | 77 | −1.7 | −7.9 | 90 |
| 1lp1b_003_I13A_L42A | I13A L42A | HEK293 | >10,000 | −25.6 | 18.8 | 10,700 |
| 1lp1b_003_L19A | L19A | HEK293 | >10,000 | −27.3 | 20.9 | 21,000 |
| 1lp1b_003_L19A_L41I | L19A L41I | HEK293 | >10,000 | | | |
| 1lp1b_003_I13A | I13A | HEK293 | 2300 | −16.2 | 7.4 | 380 |
| 1lp1b_003_L16A | L16A | HEK293 | 2300 | −12.3 | 3.4 | 341 |
| 1lp1b_003_F27A | F27A | HEK293 | 490 | −12.4 | 2.7 | 83 |
| 1lp1b_003_L41A | L41A | HEK293 | 310 | −4.0 | −5.7 | 79 |
| 1lp1b_003_L42A | L42A | HEK293 | 218 | −4.4 | −5.1 | 93 |
| 1lp1b_003_ferritin | 1lp1b_ferritin | HEK293 | | | | |
| 1lp1b_003_eumS | eumS | HEK293 | | | | |
| 1lp1b_003_eumSP | eumSP | HEK293 | | | | |
| 1lp1b_003_eumL | eumL | HEK293 | | | | |
| 1lp1b_003_eumLP | eumLP | HEK293 | | | | |
| 1lp1b_003_Neg1 | Neg1 | HEK293 | | | | |

The results indicate that a number of the immunogens bind motavizumab with a K_{d} within approximately an order of magnitude of the K_{d} of RSV F peptide for motavizumab (K_{d} of ˜250 nM). Immunogen 1lp1b_{—}003_K272E has a mutation resulting in removal of a key contact residue within the motavizumab-binding domain; as such, that immunogen would not be expected to bind to motavizumab.

### Example 8

### Immunogenicity Data: Binding and Neutralization

This Example demonstrates the ability of immunogens of the embodiments to elicit a humoral immune response that yields immune sera capable of binding to F protein, F peptide (having SEQ ID NO:2), and scaffold immunogens. The Example further demonstrates the ability of immunogens of the embodiments to elicit a neutralizing humoral immune response against RSV.

Mice were immunized as follows: Six- to eight-week old female C57BL/6 mice (Jackson Laboratory, Bar Harbor, Me.) were used for all experiments. Mice were immunized with immunogen and 25 μg CpG/mouse intramuscularly. Table 9 indicates which immunogens were tested, as well as the immunogen doses, administration regimen (including identification of immunogen administered as a prime (dose 1), identification of immunogen(s) administered as a boost(s), and number of boosts (i.e., dose 2, dose 3, etc.), interval of time between doses, and number of mice used. Please note that the immunogen names in Table 9 are an abbreviated form of immunogens listed in Table 8; Table 8 provides both names. Table 9 also indicates if the immunogens did not include a PADRE motif (-PADRE). Sera were collected on day 10 or day 14 and tested as described herein.

The abilities of the murine immune sera to bind 1lp1b_{—}003 immunogen (labeled as 1lp1b in Table 9), 1lp1b(K272E) protein (the mutation causing loss of motavizumab binding, and labeled as 1lp1b(K272E) in Table 9), RSV F peptide, and RSV F protein were tested using a kinetic ELISA as follows: The RSV F protein and scaffold proteins were diluted in PBS and coated onto 96-well flat bottom ELISA plates at a concentration of 1 μg/ml and incubated overnight at 4° C. For RSV F peptide binding, a biotinylated RSV F peptide (biotin-peptide) was used; the biotin-peptide was coated onto a Neutravidin plate (Thermo Scientific, Rockford, Ill.) and incubated for 2 hours at room temperature. For all samples, nonspecific adsorption was prevented with 200 μL/well of blocking buffer (2% BSA in PBS) for 1 hour at 37° C. Plates were then washed four times on an automated plate washer (Bio-Tek Instruments, Winooski, Vt.) with wash buffer (0.02% Tween-20 in PBS). One hundred μL of diluted test sera (1:100 in blocking buffer) or positive serum control were added to each well. Plates were incubated for one hour at room temperature, washed four times, and incubated for 1 hour at room temperature with HRP-conjugated goat anti-mouse IgG antibody (Jackson ImmunoResearch Laboratories, West Grove, Pa.). Plates were washed with wash buffer four times followed by distilled water. One hundred μl of Super AquaBlue ELISA substrate (eBioscience, San Diego, Calif.) was added to each well, and plates were read immediately using a Dynex Technologies microplate reader (Chantilly, Va.). The rate of color change in mOD/min was read at a wavelength of 405 nm every 9 seconds for 5 minutes with the plates shaken before each measurement. The mean mOD/min reading of duplicate wells was calculated, and the background mOD/min was subtracted from the corresponding control well.

Neutralization activity was measured using a flow cytometric neutralization assay as described in the Examples herein.

The abilities of immune sera elicited by immunogens of the embodiments to bind to RSV F protein and RSV F peptide are indicated in Table 9. Table 9 also provides data from the neutralization assay, expressed as number of mice (between 0 and 5) showing a specified result (Frequency) and reciprocal dilution of immune sera at which EC₅₀ was achieved (Magnitude). For certain immunogens, the immune sera, while not achieving 50% neutralization under the stipulated conditions, did show lower amounts of neutralization. Those results are shown in the last column (Comments). These data are expressed as % of neutralization at a specified dilution of sera. For example, “40% neutralization in 1:10” means that all 5 mice showed 40% neutralization when the respective immune sera were diluted by a factor of 10. “3/5 20% neutralization in 1:10” means that 3 of the 5 mice showed 20% neutralization when the respective immune sera were diluted by a factor of 10. A blank square indicates that the respective immunogen was not tested in that assay. “nd” means not detectable; i.e., below the limit of detection of the assay.

TABLE 9
Immunogenicity of immune sera elicited by immunogens

| | | | | | | | | | | | Neutral- | | |
|---|---|---|---|---|---|---|---|---|---|---|---|---|---|
| | Immuno- | | | | | | | | | | ization | | |
| | gen | | | | | | | 1lp1b | RSV F | RSV F | assay | | |
| | Dose | | | | | | 1lp1b | (K272E) | Peptide | protein | (flow | | |
| | (immuno- | | | | | | binding | binding | binding | binding | cytometry) | | |
| | gen + | | | | | | (kinetic | (kinetic | (kinetic | (kinetic | Fre- | Mag- | |
| | 25 μg | | | | | | ELISA) | ELISA) | ELISA) | ELISA) | quen- | ni- | |
| Trial | CpG) | dose 1 | dose 2 | dose 3 | Interval | N | mean | mean | mean | mean | cy | tude | Comments |
| 2 | 10 μg | 1lp1b | 1lp1b | | 3 weeks | 5 | 75.8 | 67.4 | | nd | nd | nd | |
| | 10 μg | 1lp1b(−) | 1lp1b(−) | | | 5 | 2.8 | 2.3 | | 0.2 | nd | nd | |
| | | PADRE | PADRE | | | | | | | | | | |
| | 10 μg | 1s2xa | 1s2xa | | | 5 | 0.2 | 0.8 | | 1.2 | nd | nd | |
| | | (E. coli) | (E. coli) | | | | | | | | | | |
| | 10 μg | 1lp1b | 1lp1b | | | 5 | 0.4 | 0.2 | | 0.4 | nd | nd | |
| | | (E. coli) | (E. coli) | | | | | | | | | | |
| | 10 μg | 1lp1b | 1lp1b | 1lp1b | 4 weeks | 5 | 113 | 88 | | nd | nd | nd | |
| | 10 μg | 1lp1b | 1lp1b | 1lp1b | | 5 | 3.8 | 4.2 | | nd | nd | nd | |
| | 10 μg | 1s2xa | 1s2xa | 1s2xa | | 5 | 0.2 | 0.4 | | nd | nd | nd | |
| | | (E. coli) | (E. coli) | (E. coli) | | | | | | | | | |
| | 10 μg | 1lp1b | 1lp1b | 1lp1b | | 5 | 1 | nd | | 2.21 | nd | nd | |
| | | (E. coli) | (E. coli) | (E. coli) | | | | | | | | | |
| 3 | 20 μg | 1lp1b_Ferritin | 1lp1b_Ferritin | 1lp1b_Ferritin | 2 weeks | 5 | 12 | 15.33 | | | nd | nd | |
| | 20 μg | 1lp1b_Glyc 1 | 1lp1b_Glyc 1 | 1lp1b_Glyc 1 | | 5 | 3.6 | 2 | | | nd | nd | |
| | 20 μg | 1lp1b_surf 1 | 1lp1b_Glyc 1 | 1lp1b_surf 1 | | 5 | 0.6 | 1 | | | nd | nd | |
| | 20 μg | 1lp1b_surf 8 | 1lp1b_Glyc 1 | 1lp1b_surf 8 | | 5 | 0.8 | 0.4 | | | nd | nd | |
| 4 | 20 μg | 1lp1b | 1lp1b | | 2 weeks | 5 | | | 1.8 | 2.2 | nd | nd | |
| | 20 μg | L41A | L41A | | | 5 | | | 0.4 | 1.6 | nd | nd | |
| | 20 μg | L42A | L42A | | | 5 | | | 0 | 2.6 | nd | nd | |
| | 20 μg | Glyc 2 | Glyc 2 | | | 5 | | | 2.6 | 0.8 | nd | nd | |
| | 20 μg | 113L F27A | 113L F27A | | | 5 | | | 0.4 | 1.4 | nd | nd | |
| | 20 μg | 113L F27A | L42A | | | 5 | | | 0.4 | 1 | nd | nd | Additional |
| | | | | | | | | | | | | | Neutral- |
| | | | | | | | | | | | | | ization |
| | | | | | | | | | | | | | Results: |
| | 20 μg | 1lp1b | 1lp1b | 1lp1b- | 2 weeks | 5 | 18.8 | 19 | 0.2 | 1.8 | 2 | 15-20 | 40% |
| | | | | Ferritin | | | | | | | | | neutral- |
| | | | | | | | | | | | | | ization |
| | | | | | | | | | | | | | in 1:10 |
| | 20 μg | L41A | L41A | 1lp1b- | | 5 | 15.2 | 15.6 | 0.2 | 1.2 | 2 | 10-17 | 40% |
| | | | | Ferritin | | | | | | | | | neutral- |
| | | | | | | | | | | | | | ization |
| | | | | | | | | | | | | | in 1:10 |
| | 20 μg | L42A | L42A | 1lp1b- | | 5 | 12.8 | 10.2 | nd | 2.2 | 2 | 15 | 40% |
| | | | | Ferritin | | | | | | | | | neutral- |
| | | | | | | | | | | | | | ization |
| | | | | | | | | | | | | | in 1:10 |
| | 20 μg | Glyc 2 | Glyc 2 | 1lp1b- | | 5 | 1.8 | 1.8 | 0.6 | 0.4 | 2 | 10-13 | 40% |
| | | | | Ferritin | | | | | | | | | neutral- |
| | | | | | | | | | | | | | ization |
| | | | | | | | | | | | | | in 1:10 |
| | 20 μg | 113L F27A | 113L F27A | 1lp1b- | | 5 | 6.2 | 5.4 | nd | 0.6 | 2 | 10 | 40% |
| | | | | Ferritin | | | | | | | | | neutral- |
| | | | | | | | | | | | | | ization |
| | | | | | | | | | | | | | in 1:10 |
| | 20 μg | 113L F27A | L42A | Glyc 2 | | 5 | 11.4 | 11.4 | 0.4 | 0.8 | 3 | 9-15 | 40% |
| | | | | | | | | | | | | | neutral- |
| | | | | | | | | | | | | | ization |
| | | | | | | | | | | | | | in 1:10 |
| 5 | 20 μg | k46A | k46A | 1lp1b_Ferritin | 2 weeks | 5 | 14.8 | 6.6 | nd | 1.75 | 4 | 10-20 | |
| | 20 μg | q52A | q52A | 1lp1b- | | 5 | 7.8 | 7.2 | nd | 3.6 | 4 | 11-20 | |
| | | | | Ferritin | | | | | | | | | |
| | 20 μg | L141L/L42V | L141L/L42V | 1lp1b- | | 5 | 19 | 12.8 | nd | 2.75 | 3 | 10-16 | |
| | | | | Ferritin | | | | | | | | | |
| | 20 μg | eumS | eumS | 1lp1b- | | 5 | 12 | 11.6 | nd | 2.2 | 3 | 10-24 | |
| | | | | Ferritin | | | | | | | | | |
| | 20 μg | eumSP | eumSP | 1lp1b- | | 5 | 25.4 | 22.8 | nd | 3 | 3 | 10-20 | |
| | | | | Ferritin | | | | | | | | | |
| | 20 μg | eumL | eumL | 1lp1b- | | 5 | 15.4 | 14.8 | 0.4 | 3.4 | nd | ~10 | 50% |
| | | | | Ferritin | | | | | | | | | neutral- |
| | | | | | | | | | | | | | ization |
| | | | | | | | | | | | | | in 1:10 |
| | 20 μg | eumLP | eumLP | 1lp1b- | | 5 | 13.5 | 15 | 0.2 | 2.8 | 4 | 13-27 | |
| | | | | Ferritin | | | | | | | | | |
| 6 | 20 μg | L16A | L16A | L16A | 2 weeks | 5 | 13 | 15.4 | 0.6 | 1.4 | nd | nd | 3/5 20% |
| | | | | | | | | | | | | | neutral- |
| | | | | | | | | | | | | | ization |
| | | | | | | | | | | | | | in 1:10 |
| | 20 μg | Neg 1 | Neg 1 | Neg 1 | | 5 | 10.6 | 14.2 | 0.2 | 3 | nd | nd | 3/4 50% |
| | | | | | | | | | | | | | neutral- |
| | | | | | | | | | | | | | ization |
| | | | | | | | | | | | | | in 1:10 |
| | 20 μg | 113A | 113A | 113A | | 5 | 1.8 | 2 | 0.4 | 1.2 | nd | nd | 3/5 35% |
| | | | | | | | | | | | | | neutral- |
| | | | | | | | | | | | | | ization |
| | | | | | | | | | | | | | in 1:10 |
| | 20 μg | F27A | F27A | F27A | | 5 | 3 | 2.2 | 0.2 | 2.8 | nd | nd | 3/5 25% |
| | | | | | | | | | | | | | neutral- |
| | | | | | | | | | | | | | ization |
| | | | | | | | | | | | | | in 1:10 |

| | | | | | | | | 1lp1b | RSV F | RSV F | | |
|---|---|---|---|---|---|---|---|---|---|---|---|---|
| | | | | | | | 1lp1b | (K272E) | Peptide | protein | | |
| | | | | | | | binding | binding | binding | binding | | |
| PROSCI | | | | | | | (kinetic | (kinetic | (kinetic | (kinetic | Neutralization assay | |
| Trial | Vaccination | dose 1, 2 | dose 3, 4 | dose 5, 6 | Interval | N | ELISA) | ELISA) | ELISA) | ELISA) | (flow cytometry) | |
| 1139 | 20 μg | 2X 1lp1b | 2X 1lp1b | 2X 1lp1b_Ferritin | 2 weeks | 5 | | | nd | 4.6 | nd | nd |
| 1140 | scaffold + | 2X 1lp1b | 2X 1s2xa | 2X 1lp1b_Ferritin | | 5 | | | 4.25 | 4.75 | nd | nd |
| | 50 μg CpG- | | (CagZ) | | | | | | | | | |
| 1141 | 25 μl in 2% | 2X 1lp1b | 2X | | | 5 | | | nd | 59 | nd | nd |
| | alum | | 1lpb1_surf1 | | | | | | | | | |
| 1142 | | 2X 1s2xa | 2X 1s2xa | | | 5 | | | 0.4 | 1.4 | nd | nd |
| | | (CagZ) | (CagZ) | | | | | | | | | |
| 1143 | | 2X 1s2xa | 2X 1lp1b | | | 5 | | | 0.2 | 3.8 | nd | nd |
| | | (CagZ) | | | | | | | | | | |
| 1144 | | 2X pepide | 2X 1lp1b | | | 5 | | | 49.8 | 9.4 | nd | nd |

The results in Table 9 indicate that immune sera elicited by immunogens of the embodiments demonstrated binding to 1lp1b_{—}003 (referred to in Table 9 as 1lp1b) under the conditions tested. Some binding was also observed to 1lp1b_{—}003_K272E under the conditions tested. A number of the immune sera also bound to RSV F peptide or RSV F protein under the conditions tested. Interestingly, immune sera from all 5 mice immunized twice with 1lp1b_{—}003 followed by two boosts with 1lp1b_{—}003_Surf1 (trial 1141) showed high binding to RSV F protein. Some of the immunogens also elicited a neutralizing humoral immune response. Interestingly, boosting with a multivalent immunogen comprising ferritin stimulated a neutralizing humoral immune response. It is to be appreciated that while a number of immunogen immunizations led to undetectable neutralization, those results do not preclude other prime and/or boost conditions being found that could enable neutralization with such immunogens.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims.

1. An RSV immunogen comprising an amino acid sequence of 1LP1_b (SEQ ID NO: 11) having from one to twenty amino acid substitutions, wherein at least one amino acid substitution is selected from the group consisting of:
(a) substitution of a serine at amino acid position N-25 in SEQ ID NO:11;
(b) substitution of a leucine at amino acid position I-28 in SEQ ID NO:11;
(c) substitution of a serine at amino acid position Q-29 in SEQ ID NO: 11;
(d) substitution of an isoleucine at amino acid position L-31 in SEQ ID NO:11;
(e) substitution of an asparagine at amino acid position K-32 in SEQ ID NO:11;
(f) substitution of an aspartic acid at amino acid position K-4 in SEQ ID NO:11;
(g) substitution of a lysine at amino acid position Q-6 in SEQ ID NO:11;
(h) substitution of a lysine at amino acid position Q-7 in SEQ ID NO:11;
(i) substitution of a leucine at amino acid position N-8 in SEQ ID NO:11;
(j) substitution of a serine at amino acid position F-10 in SEQ ID NO:11; and
(k) substitution of an asparagine at amino acid position Y-11 in SEQ ID NO:11.

2. (canceled)

3. The RSV immunogen of claim 1, wherein said RSV immunogen comprises an amino acid sequence that is at least 95% identical to an amino acid sequence of a protein selected from the group consisting of 1lp1b_{—}001 (SEQ ID NO:18), 1lp1b_{—}002 (SEQ ID NO:21), 1lp1b_{—}003 (SEQ ID NO:24), and 1lp1b_{—}004 (SEQ ID NO:149).

4. The RSV immunogen of claim 1, wherein said RSV immunogen comprises an amino acid sequence of a protein selected from the group consisting of 1lp1b_{—}001 (SEQ ID NO:18), 1lp1b_{—}002 (SEQ ID NO:21), 1lp1b_{—}003 (SEQ ID NO:24), 1lp1b_{—}004 (SEQ ID NO:149), mota_{—}1lp1b.m1.c1.d1_glyc1 (SEQ ID NO:55), mota_{—}1lp1b.m1.c1.d1_glyc2 (SEQ ID NO:56), 1lp1b_{—}003_Glyc1 (SEQ ID NO:39), 1lp1b_{—}003_Glyc2 (SEQ ID NO:42), 1lp1b_{—}003_Glyc3 (SEQ ID NO:45), 1lp1b_{—}003_Glyc4 (SEQ ID NO:48), 1lp1b_{—}003_Glyc5 (SEQ ID NO:51), 1lp1b_{—}003_Glyc6 (SEQ ID NO:54), mota_{—}1lp1b.m1.c1.d1_des1_{—}1 (SEQ ID NO:66), mota_{—}1lp1b.m1.c1.d1_des1_{—}2 (SEQ ID NO:67), mota_{—}1lp1b.m1.c1.d1_des1_{—}3 (SEQ ID NO:68 mota_{—}1lp1b.m1.c1.d1_des1_{—}5 (SEQ ID NO:69), mota_{—}1lp1b.m1.c1.d1_des1_{—}6 (SEQ ID NO:70), mota_{—}1lp1b.m1.c1.d1_des1_{—}7 (SEQ ID NO:71), mota_{—}1lp1b.m1.c1.d1_des1_{—}8 (SEQ ID NO:72), mota_{—}1lp1b.m1.c1.d1_des1_{—}9 (SEQ ID NO:73), mota_{—}1lp1b.m1.c1.d1_des1_{—}10 (SEQ ID NO:74), mota_{—}1lp1b.m1.c1.d1_des1_{—}11 (SEQ ID NO:75), 1lp1b_{—}003_Surf1 (SEQ ID NO:59), 1lp1b_{—}003_Surf6 (SEQ ID NO:62), 1lp1b_{—}003_Surf8 (SEQ ID NO:65), 1lp1b_{—}003_ferritin (SEQ ID NO:138), 1lp1b_{—}003_eumS (SEQ ID NO:140), 1lp1b_{—}003_eumSP (SEQ ID NO:142), 1lp1b_{—}003_eumL (SEQ ID NO:144), 1lp1b_{—}003_eumLP (SEQ ID NO:146), 1lp1b_{—}003_K46A (SEQ ID NO:78), 1lp1b_{—}003_Q52A (SEQ ID NO:81), 1lp1b_{—}003_I13L_F27A (SEQ ID NO:87), 1lp1b_{—}003_L41I_L42V (SEQ ID NO:90), 1lp1b_{—}003_L41I_L42A (SEQ ID NO:93), lp1b_{—}003_I13A (SEQ ID NO:105), 1lp1b_{—}003_L16A (SEQ ID NO:108), 1lp1b_{—}003_F27A (SEQ ID NO:111), 1lp1b_{—}003_L41A (SEQ ID NO:114), 1lp1b_{—}003_L42A (SEQ ID NO:117), and 1lp1b_{—}003_Neg1 (SEQ ID NO:132).

5-10. (canceled)

11. An RSV immunogen comprising an amino acid sequence of truncated 1S2X_a (SEQ ID NO:13) having from one to twenty-five amino acid substitutions, wherein at least one amino acid substitution is selected from the group consisting of:
(a) substitution of a serine at amino acid position 92 in SEQ ID NO:13;
(b) substitution of a leucine at amino acid position 95 in SEQ ID NO:13;
(c) substitution of a serine at amino acid position 96 in SEQ ID NO:13;
(d) substitution of an isoleucine at amino acid position 98 in SEQ ID NO:13;
(e) substitution of an asparagine at amino acid position 99 in SEQ ID NO:13;
(f) substitution of an aspartic acid at amino acid position 100 in SEQ ID NO:13;
(g) substitution of an asparagine at amino acid position 105 in SEQ ID NO:13;
(h) substitution of an aspartic acid at amino acid position 106 in SEQ ID NO:13;
(i) substitution of a lysine at amino acid position 108 in SEQ ID NO:13;
(j) substitution of a lysine at amino acid position 109 in SEQ ID NO:13;
(k) substitution of a leucine at amino acid position 110 in SEQ ID NO:13;
(l) substitution of a serine at amino acid position 112 in SEQ ID NO:13; and
(m) substitution of an asparagine at amino acid position 113 in SEQ ID NO:13.

12. (canceled)

13. The RSV immunogen of claim 11, wherein said RSV immunogen comprises an amino acid sequence that is at least 95% identical to an amino acid sequence of a protein selected from the group consisting of 1s2xa_{—}001 (SEQ ID NO:152), 1s2xa_{—}002 (SEQ ID NO:155), 1s2xa_{—}003 (SEQ ID NO:158), and 1s2xa_{—}004 (SEQ ID NO:164).

14. The RSV immunogen of claim 11, wherein said RSV immunogen comprises an amino acid sequence of a protein selected from the group consisting of 1s2xa_{—}001 (SEQ ID NO:152), 1s2xa_{—}002 (SEQ ID NO:155), 1s2xa_{—}003 (SEQ ID NO:158), and 1s2xa_{—}004 (SEQ ID NO:164).

15. The RSV immunogen of claim 1, wherein said RSV immunogen comprises at least one characteristic selected from the group consisting of binding a motavizumab antibody, eliciting a humoral immune response against RSV and failing to elicit a cellular immune response.

16-18. (canceled)

19. The RSV immunogen of claim 1, wherein said RSV immunogen has a motavizumab antibody-binding domain comprising less than 9 consecutive amino acids from a motavizumab antibody-binding domain of RSV fusion protein.

20. A nucleic acid molecule comprising a nucleic acid sequence that encodes an immunogen of claim 11.

21. A nucleic acid molecule comprising a nucleic acid sequence that encodes an immunogen of claim 1.

22-26. (canceled)

27. A method selected from the group consisting of a method to elicit a neutralizing humoral immune response against RSV and a method to protect a patient from RSV infection, said method comprising administering an RSV immunogen of claim 1.

28. (canceled)

29. An immunogen comprising an antibody-binding domain that binds an antibody selected from the group consisting of motavizumab and 101F antibody,
wherein the three-dimensional structure of said antibody-binding domain of said immunogen spatially corresponds to a three-dimensional structure of an antibody-binding domain of a fusion (F) peptide derived from respiratory syncytial virus (RSV) fusion (F) protein in a complex selected from the group consisting of:
(a) a complex between a F peptide consisting of amino acid sequence SEQ ID NO:2 and motavizumab, said complex being set forth in the three-dimensional model defined by the atomic coordinates specified in Protein Data Bank accession code 3IXT; and
(b) a complex between a F peptide consisting of amino acid sequence SEQ ID NO:4 and 101F antibody, said complex being set forth in the three-dimensional model defined by the atomic coordinates specified in Protein Data Bank accession code 3O41;
wherein said antibody-binding domain of said immunogen comprises less than 12 consecutive amino acids from a motavizumab-binding domain or a 101F antibody-binding domain of RSV F protein, and wherein said immunogen elicits a humoral immune response against RSV.

30. The immunogen of claim 29, wherein said antibody-binding domain of said immunogen comprises contact residues selected from the group consisting of:
(a) contact residues that have a spatial orientation represented by atomic coordinates that have a root mean square deviation of protein backbone atoms of less than 10 angstroms from the corresponding backbone atoms of contact residues in an RSV F peptide that contacts motavizumab or 101F antibody in a complex set forth in claim 29;
(b) contact residues of the motavizumab-binding domain embedded in a protein scaffold comprising a three-dimensional structure having two alpha helices defined by atomic coordinates that have a root mean square deviation of protein backbone atoms of less than 10 angstroms when superimposed on the two alpha helices of the peptide consisting of amino acid sequence SEQ ID NO:2 when complexed with motavizumab, the three-dimensional model of said complex being defined by the coordinates specified in Protein Data Bank accession code 3IXT; and,
(c) contact residues of the 101F antibody-binding domain embedded in a protein scaffold comprising a three-dimensional structure with atomic coordinates that have a root mean square deviation of protein backbone atoms of less than 10 angstroms when superimposed on the peptide consisting of amino acid sequence SEQ ID NO:4 when complexed with 101F antibody, the 3-dimensional model of said complex being defined by the coordinates specified in Protein Data Bank accession code 3O41.

31-33. (canceled)

34. An RSV immunogen of claim 29, wherein said antibody-binding domain of said immunogen comprises less than 9 consecutive amino acids from a motavizumab-binding domain or a 101F antibody-binding domain of RSV F protein.

35. The immunogen of claim 29, wherein the motavizumab-binding domain of said immunogen comprises less than 15 amino acids of the motavizumab-binding domain from said RSV F peptide, wherein said amino acids are in clusters of no more than 8 consecutive amino acids per cluster.

36. (canceled)

37. The immunogen of claim 29, wherein the 101F antibody-binding domain of said immunogen comprises no more than 10 amino acids of the 101F antibody-binding domain of said RSV F peptide, wherein said amino acids are in clusters of no more than 8 consecutive amino acids per cluster.

38. (canceled)

39. The immunogen of claim 29, wherein said immunogen comprises an amino acid sequence of a protein selected from the group consisting of 1LP1_b (SEQ ID NO:11), 1S2X_a (SEQ ID NO:12), truncated 1S2X_a (SEQ ID NO:13), and 2EIA_a (SEQ ID NO:14).

40. The immunogen of claim 29, wherein said immunogen comprises an amino acid sequence selected from the group consisting of 1LP1_b (SEQ ID NO:11), 1S2X_a (SEQ ID NO:12), truncated 1S2X_a (SEQ ID NO:13), and 2EIA_a (SEQ ID NO:14), wherein said amino acid sequence comprises at least one substitution selected from the group consisting of:
(a) substitution of a serine for the amino acid spatially corresponding to the amino acid at position 2 of SEQ ID NO:2;
(b) substitution of a leucine for the amino acid spatially corresponding to the amino acid at position 5 of SEQ ID NO:2;
(c) substitution of a serine for the amino acid spatially corresponding to the amino acid at position 6 of SEQ ID NO:2;
(d) substitution of an isoleucine for the amino acid spatially corresponding to the amino acid at position 8 of SEQ ID NO:2;
(e) substitution of an asparagine for the amino acid spatially corresponding to the amino acid at position 9 of SEQ ID NO:2;
(f) substitution of an aspartic acid for the amino acid spatially corresponding to the amino acid at position 10 of SEQ ID NO:2;
(g) substitution of a threonine for the amino acid spatially corresponding to the amino acid at position 14 of SEQ ID NO:2;
(h) substitution of an asparagine for the amino acid spatially corresponding to the amino acid at position 15 of SEQ ID NO:2;
(i) substitution of an aspartic acid for the amino acid spatially corresponding to the amino acid at position 16 of SEQ ID NO:2;
(j) substitution of a lysine for the amino acid spatially corresponding to the amino acid at position 18 of SEQ ID NO:2;
(k) substitution of a lysine for the amino acid spatially corresponding to the amino acid at position 19 of SEQ ID NO:2;
(l) substitution of a leucine for the amino acid spatially corresponding to the amino acid at position 20 of SEQ ID NO:2;
(m) substitution of a serine for the amino acid spatially corresponding to the amino acid at position 22 of SEQ ID NO:2; and
(n) substitution of an asparagine for the amino acid spatially corresponding to the amino acid at position 23 of SEQ ID NO:2.

41. (canceled)

42. The immunogen of claim 30, wherein said protein scaffold comprises an amino acid sequence comprising at least one substitution selected from the group consisting of:
(a) substitution of a lysine for the amino acid spatially corresponding to the amino acid at position 1 of SEQ ID NO:4;
(b) substitution of an arginine for the amino acid spatially corresponding to the amino acid at position 3 of SEQ ID NO:4;
(c) substitution of a isoleucine for the amino acid spatially corresponding to the amino acid at position 5 of SEQ ID NO:4;
(d) substitution of a isoleucine for the amino acid spatially corresponding to the amino acid at position 6 of SEQ ID NO:4;
(e) substitution of a lysine for the amino acid spatially corresponding to the amino acid at position 7 of SEQ ID NO:4;
(f) substitution of a threonine for the amino acid spatially corresponding to the amino acid at position 8 of SEQ ID NO:4;
(g) substitution of a phenylalanine for the amino acid spatially corresponding to the amino acid at position 9 of SEQ ID NO:4; and,
(h) substitution of a serine for the amino acid spatially corresponding to the amino acid at position 10 of SEQ ID NO: 4.

43. (canceled)

44. The immunogen of claim 29, wherein said immunogen comprises an amino acid sequence of a protein selected from the group consisting of 1lp1b_{—}001 (SEQ ID NO:18), 1lp1b_{—}002 (SEQ ID NO:21), 1lp1b_{—}003 (SEQ ID NO:24), 1lp1b_{—}004 (SEQ ID NO:149), 1s2xa_{—}001 (SEQ ID NO:152), 1s2xa_{—}002 (SEQ ID NO:155), 1s2xa_{—}003 (SEQ ID NO:158), 1s2xa_{—}004 (SEQ ID NO:164), 2eiaa_{—}001 (SEQ ID NO:167), 2eiaa_{—}002 (SEQ ID NO:170), mota_{—}1lp1b.m1.c1.d1_glyc1 (SEQ ID NO:55), mota_{—}1lp1b.m1.c1.d1_glyc2 (SEQ ID NO:56), 1lp1b_{—}003_Glyc1 (SEQ ID NO:39), 1lp1b_{—}003_Glyc2 (SEQ ID NO:42), 1lp1b_{—}003_Glyc3 (SEQ ID NO:45), 1lp1b_{—}003_Glyc4 (SEQ ID NO:48), 1lp1b_{—}003_Glyc5 (SEQ ID NO:51), 1lp1b_{—}003_Glyc6 (SEQ ID NO:54), mota_{—}1lp1b.m1.c1.d1_des1_{—}1 (SEQ ID NO:66), mota_{—}1lp1b.m1.c1.d1_des1_{—}2 (SEQ ID NO:67), mota_{—}1lp1b.m1.c1.d1_des1_{—}3 (SEQ ID NO:68), mota_{—}1lp1b.m1.c1.d1_des1_{—}5 (SEQ ID NO:69), mota_{—}1lp1b.m1.c1.d1_des1_{—}6 (SEQ ID NO:70), mota_{—}1lp1b.m1.c1.d1_des1_{—}7 (SEQ ID NO:71), mota_{—}1lp1b.m1.c1.d1_des1_{—}8 (SEQ ID NO:72), mota_{—}1lp1b.m1.c1.d1_des1_{—}9 (SEQ ID NO:73), mota_{—}1lp1b.m1.c1.d1_des1_{—}10 (SEQ ID NO:74), mota_{—}1lp1b.m1.c1.d1_des1_{—}11 (SEQ ID NO:75), 1lp1b_{—}003_Surf1 (SEQ ID NO:59), 1lp1b_{—}003_Surf6 (SEQ ID NO:62), and 1lp1b_{—}003_Surf8 (SEQ ID NO:65), 1lp1b_{—}003_ferritin (SEQ ID NO:138), 1lp1b_{—}003_eumS (SEQ ID NO:140), 1lp1b_{—}003_eumSP (SEQ ID NO:142), 1lp1b_{—}003_eum_L (SEQ ID NO:144), 1lp1b_{—}003eum_LP (SEQ ID NO:146), 1lp1b_{—}003_K46A (SEQ ID NO:78), 1lp1b_{—}003_Q52A (SEQ ID NO:81), 1lp1b_{—}003_I13L_F27A (SEQ ID NO:87), 1lp1b_{—}003_L41I_L42V (SEQ ID NO:90), 1lp1b-003_L41I_L42A (SEQ ID NO:93), 1lp1b_{—}003_I13A (SEQ ID NO:105), 1lp1b_{—}003_L16A (SEQ ID NO:108), 1lp1b_{—}003_F27A (SEQ ID NO:111), 1lp1b_{—}003_L41A (SEQ ID NO:114), 1lp1b_{—}003_L42A (SEQ ID NO:117), and 1lp1b_{—}003_Neg1 (SEQ ID NO:132).

45-49. (canceled)

50. The RSV immunogen of claim 29, wherein said immunogen elicits a humoral immune response against RSV, but not a cellular immune response.

51. An immunogen comprising a protein comprising an amino acid sequence of a protein selected from the group consisting of RSV F₀ Fd (SEQ ID NO:174), RSV F Fd (SEQ ID NO:175), and RSV F0 Fd GAG (SEQ ID NO:176).

52. A nucleic acid molecule comprising a nucleic acid sequence that encodes an immunogen of claim 29.

53-59. (canceled)

60. A method selected from the group consisting of:
(a) a method to elicit a neutralizing humoral immune response against RSV and,
(b) a method to protect a patient from RSV infection, said method comprising administering an RSV immunogen of claim 29.

61. (canceled)

62. An antibody protein selected from the group consisting of:
A) an antibody protein comprising a heavy chain comprising SEQ ID NO:5, except that said antibody protein comprises at least one amino acid substitution selected from the group consisting of:
(a) substitution of the amino acid corresponding to position 32 of SEQ ID NO:5, is a histidine or a glutamic acid;
(b) substitution of the amino acid at position 35 of SEQ ID NO:5 is substituted with an alanine;
(c) substitution of the amino acid at position 52 of SEQ ID NO:5 is substituted with an amino acid selected from the group consisting of lysine, histidine, threonine, serine and arginine;
(d) substitution of the amino acid at position 53 of SEQ ID NO:5 is substituted with a histidine or a serine;
(e) substitution of the amino acid at position 54 of SEQ ID NO:5 is substituted with a phenylalanine or an arginine;
(f) substitution of the amino acid at position 56 of SEQ ID NO:5 is substituted with an amino acid selected from the group consisting of isoleucine, serine, glutamic acid, and aspartic acid;
(g) substitution of the amino acid at position 58 of SEQ ID NO:5 is substituted with a tyrosine or a tryptophan;
(h) substitution of the amino acid at position 97 of SEQ ID NO:5 is substituted with an aspartic acid, a histidine or an arginine;
(i) substitution of the amino acid at position 99 of SEQ ID NO:5 is substituted with aspartic acid;
(j) substitution of the amino acid at position 100 of SEQ ID NO:5 is substituted with a tyrosine, a tryptophan or a histidine; and
(k) substitution of the amino acid at position 100A of SEQ ID NO:5 is substituted with a serine or a threonine; and,
B). an antibody protein comprising a heavy chain comprising SEQ ID NO:6, except that said antibody protein comprises at least one amino acid substitution selected from the group consisting of:
(a) substitution of the amino acid at position 32 of SEQ ID NO:6 is substituted with a phenylalanine;
(b) substitution of the amino acid at position 49 of SEQ ID NO:6 is substituted with a histidine or an arginine;
(c) substitution of the amino acid at position 92 of SEQ ID NO:6 is substituted with a lysine;
(d) substitution of the amino acid at position 94 of SEQ ID NO:6 is substituted with a histidine; and
(e) substitution of the amino acid at position 96 of SEQ ID NO:6 is substituted with a histidine.

63. (canceled)

64. A nucleic acid molecule comprising a nucleic acid sequence that encodes an antibody protein of claim 62.

65-68. (canceled)

69. A method to protect a patient from RSV infection comprising administering to said patient an antibody protein of claim 62, wherein said administration protects said patient from RSV infection.


SEQUENCE LISTING (188 sequences)

SEQ ID NO: 1 - protein, 513 aa, respiratory syncytial virus
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELL

SEQ ID NO: 2 - protein, 24 aa, respiratory syncytial virus
NSELLSLINDMPITNDQKKLMSNN

SEQ ID NO: 3 - protein, 15 aa, respiratory syncytial virus
STASNKNRGIIKTFS

SEQ ID NO: 4 - protein, 10 aa, respiratory syncytial virus
KNRGIIKTFS

SEQ ID NO: 5 - protein, 120 aa, Artificial Sequence [sequence omitted]

SEQ ID NO: 6 - protein, 106 aa, Artificial Sequence [sequence omitted]

SEQ ID NO: 7 - protein, 120 aa, Artificial Sequence
  synthetic construct
QVTLKESGPGILQPSQTLSLTCSFSGFSLSTSGMGVSWIRQPSGKGLEWLAHIYWDDDKRYNPSLKSRLTISKDTSRNQVFLKITSVDTADTATYYCARLYGFTYGFAYWGQGTLVTVSA

SEQ ID NO: 8 - protein, 111 aa, Artificial Sequence
  synthetic construct
DIVLTQSPASLAVSLGQRATIFCRASQSVDYNGISYMHWFQQKPGQPPKLLIYAASNPESGIPARFTGSGSGTDFTLNIHPVEEEDAATYYCQQIIEDPWTFGGGTKLEIK

SEQ ID NO: 9 - protein, 17 aa, respiratory syncytial virus
STASNKNRGIIKTFSNG

SEQ ID NO: 10 - protein, 15 aa, respiratory syncytial virus
CTASNKNRGIIKTFS

SEQ ID NO: 11 - protein, 58 aa, Staphylococcus aureus [sequence omitted]

SEQ ID NO: 12 - protein, 206 aa, Helicobacter pylori
GSPNSRVDELGFNEAERQKILDSNSSLMRNANEVRDKFIQNYATSLKDSNDPQDFLRRVQELRINMQKNFISFDAYYNYLNNLVLASYNRCKQEKTFAESTIKNELTLGEFVAEISDNFNNFTCDEVARISDLVASYLPREYLPPFIDGNMMGVAFQILGIDDFGKKLNEIVQDIGTKYIILSKNKTYLTSLERAKLITQLKLNLE

SEQ ID NO: 13 - protein, 178 aa, Helicobacter pylori
GSPNSRVDELGFNEAERQKILDSNSSLMRNANEVRDKFIQNYATSLKDSNDPQDFLRRVQELRINMQKNFISFDAYYNYLNNLVLASYNRCKQEKTFAESTIKNELTLGEFVAEISDNFNNFTCDEVARISDLVASYLPREYLPPFIDGNMMGVAFQILGIDDFGKKLNEIVQDIGTK

SEQ ID NO: 14 - protein, 206 aa, Equine infectious anemia virus
PRGYTTWVNTIQTNGLLNEASQNLFGILSVDCTSEEMNAFLDVVPGQAGQKQILLDAIDKIADDWDNRHPLPNAPLVAPPQGPIPMTARFIRGLGVPRERQMEPAFDQFRQTYRQWIIEAMSEGIKVMIGKPKAQNIRQGAKEPYPEFVDRLLSQIKSEGHPQEISKFLTDTLTIQNANEECRNAMRHLRPEDTLEEKMYACRDIG

SEQ ID NO: 15 - protein, 43 aa, respiratory syncytial virus
SAIGGYIPEAPRDGQAYVRKDGEWVLLSTFLGGIEGRHHHHHH

SEQ ID NO: 16 - DNA, 381 bp, Artificial Sequence
  synthetic construct
atgggcagcctgcagcctctggccacactgtacctgctgggcatgctggtggcctctgtgctggccgtggacaacagcttcaacgacgagaagaagctcgctagcaacgagatcgcccatctgcccaacctgaacgaggaacagcggagcgccttcctgagcagcatcaacgatgaccccagccagagcgccaatctgctggccgaggccaagaagctgaacgacgcccaggcccccaagctggaagtgctgttccagggaccaggcgctaaatttgtggccgcatggacactcaaagccgccgctggcgacgaagtggatggcagccaccaccaccatcaccacagcgcctggtcccacccccagttcgagaagtgatga

SEQ ID NO: 17 - protein, 125 aa, Artificial Sequence
  synthetic construct
MGSLQPLATLYLLGMLVASVLAVDNSFNDEKKLASNEIAHLPNLNEEQRSAFLSSINDDPSQSANLLAEAKKLNDAQAPKLEVLFQGPGAKFVAAWTLKAAAGDEVDGSHHHHHHSAWSHPQFEK

SEQ ID NO: 18 - protein, 58 aa, Artificial Sequence
  synthetic construct
VDNSFNDEKKLASNEIAHLPNLNEEQRSAFLSSINDDPSQSANLLAEAKKLNDAQAPK

SEQ ID NO: 19 - DNA, 381 bp, Artificial Sequence
  synthetic construct
atgggcagcctgcagcctctggccacactgtacctgctgggcatgctggtggcctctgtgctggccgtggacaacagcttcaacgacgagaagaagctcgctagcaacgagatccagcatctgcccaacctgaacgaggaacagcggagcgccttcatcagcagcctgaacgatgaccccagccagagcgccaatctgctggccgaggccaagaagctgaacgacgcccaggcccccaagctggaagtgctgttccagggaccaggcgctaaatttgtggccgcatggacactcaaagccgccgctggcgacgaagtggatggcagccaccaccaccatcaccacagcgcctggtcccacccccagttcgagaagtgatga

SEQ ID NO: 20 - protein, 125 aa, Artificial Sequence
  synthetic construct
MGSLQPLATLYLLGMLVASVLAVDNSFNDEKKLASNEIQHLPNLNEEQRSAFISSLNDDPSQSANLLAEAKKLNDAQAPKLEVLFQGPGAKFVAAWTLKAAAGDEVDGSHHHHHHSAWSHPQFEK

SEQ ID NO: 21 - protein, 58 aa, Artificial Sequence
  synthetic construct
VDNSFNDEKKLASNEIQHLPNLNEEQRSAFISSLNDDPSQSANLLAEAKKLNDAQAPK

SEQ ID NO: 22 - DNA, 372 bp, Artificial Sequence
  synthetic construct
atgggcagcctgcagcctctggccacactgtacctgctgggcatgctggtggcctctgtgctggccagcttcaacgacgagaagaagctcgctagcaacgagatcgcccatctgcccaacctgaacgaggaacagcggagcgccttcctgagcagcatcaacgatgaccccagccagagcgccaatctgctggccgaggccaagaagctgaacgacgcccaggcccccaagctggaagtgctgttccagggaccaggcgctaaatttgtggccgcatggacactcaaagccgccgctggcgacgaagtggatggcagccaccaccaccatcaccacagcgcctggtcccacccccagttcgagaagtgatga

SEQ ID NO: 23 - protein, 122 aa, Artificial Sequence
  synthetic construct
MGSLQPLATLYLLGMLVASVLASFNDEKKLASNEIAHLPNLNEEQRSAFLSSINDDPSQSANLLAEAKKLNDAQAPKLEVLFQGPGAKFVAAWTLKAAAGDEVDGSHHHHHHSAWSHPQFEK

SEQ ID NO: 24 - protein, 55 aa, Artificial Sequence
  synthetic construct
SFNDEKKLASNEIAHLPNLNEEQRSAFLSSINDDPSQSANLLAEAKKLNDAQAPK

SEQ ID NO: 25 - DNA, 297 bp, Artificial Sequence
  synthetic construct
atgggcagcctgcagcctctggccaccctgtacctgctgggcatgctggtggccagcgtgctcgccagctacaacgacgagaagaagctggctagcaacgagatcgccaacctgcccaacctgaacgaggaacagcggagcgccttcctgagcagcatcaacgacgaccccagccagagcgccaatctgctggccgaggccaagaagctgaacgacgcccaggccgacgaggtggacggctctcaccaccaccatcaccacagcgcctggtcccacccccagttcgagaagtgatga

SEQ ID NO: 26 - protein, 97 aa, Artificial Sequence
  synthetic construct
MGSLQPLATLYLLGMLVASVLASYNDEKKLASNEIANLPNLNEEQRSAFLSSINDDPSQSANLLAEAKKLNDAQADEVDGSHHHHHHSAWSHPQFEK

SEQ ID NO: 27 - protein, 57 aa, Artificial Sequence
  synthetic construct
SYNDEKKLASNEIANLPNLNEEQRSAFLSSINDDPSQSANLLAEAKKLNDAQADEVD

SEQ ID NO: 28 - DNA, 372 bp, Artificial Sequence
  synthetic construct
atgggcagcctgcagcctctggccacactgtacctgctgggcatgctggtggccagcgtgctcgcctcttttaacgacgagaaggaactcgcttctaacgagatcgcccatctgcccaacctgaacgaggaacagcggagcgccttcctgagcagcatcaacgatgaccccagccagagcgccaacctgctggccgaggccaagaagctgaacgacgcccaggcccccaagctggaagtgctgttccagggacctggggctaaattcgtggccgcctggactctcaaagccgccgctggcgacgaagtggatggcagccaccaccaccatcaccacagcgcctggtcccacccccagttcgagaagtgatga

SEQ ID NO: 29 - protein, 122 aa, Artificial Sequence
  synthetic construct
MGSLQPLATLYLLGMLVASVLASFNDEKELASNEIAHLPNLNEEQRSAFLSSINDDPSQSANLLAEAKKLNDAQAPKLEVLFQGPGAKFVAAWTLKAAAGDEVDGSHHHHHHSAWSHPQFEK

SEQ ID NO: 30 - protein, 55 aa, Artificial Sequence
  synthetic construct
SFNDEKELASNEIAHLPNLNEEQRSAFLSSINDDPSQSANLLAEAKKLNDAQAPK

SEQ ID NO: 31 - DNA, 372 bp, Artificial Sequence
  synthetic construct
atgggcagcctgcagcctctggccacactgtacctgctgggcatgctggtggccagcgtgctcgcctcttttaacgacgagaagaaactcgcttctaacgagatcgcccatctgcccaacctgaacgaggaacagcggagcgccttcctgagcagcatcaacgatgaccccagccagagcgccaacctgctgtgcgaggccaagaagctgaacgacgcccaggcccccaagctggaagtgctgttccagggacctggggctaaattcgtggccgcctggactctcaaagccgccgctggcgacgaagtggatggcagccaccaccaccatcaccacagcgcctggtcccacccccagttcgagaagtgatga

SEQ ID NO: 32 - protein, 122 aa, Artificial Sequence
  synthetic construct
MGSLQPLATLYLLGMLVASVLASFNDEKKLASNEIAHLPNLNEEQRSAFLSSINDDPSQSANLLCEAKKLNDAQAPKLEVLFQGPGAKFVAAWTLKAAAGDEVDGSHHHHHHSAWSHPQFEK

SEQ ID NO: 33 - protein, 55 aa, Artificial Sequence
  synthetic construct
SFNDEKKLASNEIAHLPNLNEEQRSAFLSSINDDPSQSANLLCEAKKLNDAQAPK

SEQ ID NO: 34 - DNA, 372 bp, Artificial Sequence
  synthetic construct
atgggcagcctgcagcctctggccacactgtacctgctgggcatgctggtggccagcgtgctcgcctcttttaacgacgagaagaaactcgcttctaacgagatcgcccatctgcccaacctgaacgaggaacagcggagcgccttcctgagcagcatcaacgatgaccccagccagagcgccaacctgctggccgaggccaagtgcctgaacgacgcccaggcccccaagctggaagtgctgttccagggacctggggctaaattcgtggccgcctggactctcaaagccgccgctggcgacgaagtggatggcagccaccaccaccatcaccacagcgcctggtcccacccccagttcgagaagtgatga

SEQ ID NO: 35 - protein, 122 aa, Artificial Sequence
  synthetic construct
MGSLQPLATLYLLGMLVASVLASFNDEKKLASNEIAHLPNLNEEQRSAFLSSINDDPSQSANLLAEAKCLNDAQAPKLEVLFQGPGAKFVAAWTLKAAAGDEVDGSHHHHHHSAWSHPQFEK

SEQ ID NO: 36 - protein, 55 aa, Artificial Sequence
  synthetic construct
SFNDEKKLASNEIAHLPNLNEEQRSAFLSSINDDPSQSANLLAEAKCLNDAQAPK

SEQ ID NO: 37 - DNA, 369 bp, Artificial Sequence
  synthetic construct
atgggcagcctgcagcctctggccaccctgtacctgctgggcatgctggtggccagcgtcctcgccagcttcaacgacgagaagaagctggctagcaacgagatcgcccatctgcccaacctgaacgaaacccagcggagcgccttcctgagcagcatcaacgatgaccccagccagagcgccaacctgctggccaacgccaccaagctgaacgacgcccaggcccccctggaagtgctgtttcagggccctggggccaagttcgtggccgcctggacactgaaagccgccgctggcgacgaggtggacggctctcaccaccaccatcaccacagcgcctggtcccacccccagttcgagaagtgatga

SEQ ID NO: 38 - protein, 121 aa, Artificial Sequence
  synthetic construct
MGSLQPLATLYLLGMLVASVLASFNDEKKLASNEIAHLPNLNETQRSAFLSSINDDPSQSANLLANATKLNDAQAPLEVLFQGPGAKFVAAWTLKAAAGDEVDGSHHHHHHSAWSHPQFEK

SEQ ID NO: 39 - protein, 54 aa, Artificial Sequence
  synthetic construct
SFNDEKKLASNEIAHLPNLNETQRSAFLSSINDDPSQSANLLANATKLNDAQAP

SEQ ID NO: 40 - DNA, 372 bp, Artificial Sequence
  synthetic construct
atgggcagcctgcagcctctggccaccctgtacctgctgggcatgctggtggccagcgtgctggctagcttcaacgacgagaagaagctggccagcaacgagatcgcccatctgcccaacctgaacgagacccagcggagcgccttcctgagcagcatcaacgatgaccccaaccagagcgccaacctgctggccaacgccaccaagctgaacgacgcccaggcccccaagctggaagtgctgtttcagggccctggcgccaagttcgtggccgcttggacactgaaagccgccgctggcgacgaggtggacggctctcaccaccaccatcaccacagcgcctggtcccacccccagttcgagaagtgatga

SEQ ID NO: 41 - protein, 122 aa, Artificial Sequence
  synthetic construct
MGSLQPLATLYLLGMLVASVLASFNDEKKLASNEIAHLPNLNETQRSAFLSSINDDPNQSANLLANATKLNDAQAPKLEVLFQGPGAKFVAAWTLKAAAGDEVDGSHHHHHHSAWSHPQFEK

SEQ ID NO: 42 - protein, 55 aa, Artificial Sequence
  synthetic construct
SFNDEKKLASNEIAHLPNLNETQRSAFLSSINDDPNQSANLLANATKLNDAQAPK

SEQ ID NO: 43 - DNA, 372 bp, Artificial Sequence
  synthetic construct
atgggcagcctgcagcctctggccaccctgtacctgctgggcatgctggtggccagcgtcctcgccagcttcaacgacgagaagaagctggctagcaacgagatcgcccatctgcccaacctgaacgaaacccagcggagcgccttcctgagcagcatcaacgatgaccccagccagagcgccaacctgagcgccgaggccaagaagctgaacgacaaccagacccccaagctggaagtgctgttccaggggcctggcgccaagtttgtggccgcctggacactgaaggccgctgccggcgacgaggtggacggctctcaccaccaccatcaccacagcgcctggtcccacccccagttcgagaagtgatga

SEQ ID NO: 44 - protein, 122 aa, Artificial Sequence
  synthetic construct
MGSLQPLATLYLLGMLVASVLASFNDEKKLASNEIAHLPNLNETQRSAFLSSINDDPSQSANLSAEAKKLNDNQTPKLEVLFQGPGAKFVAAWTLKAAAGDEVDGSHHHHHHSAWSHPQFEK

SEQ ID NO: 45 - protein, 55 aa, Artificial Sequence
  synthetic construct
SFNDEKKLASNEIAHLPNLNETQRSAFLSSINDDPSQSANLSAEAKKLNDNQTPK

SEQ ID NO: 46 - DNA, 372 bp, Artificial Sequence
  synthetic construct
atgggcagcctgcagcctctggccaccctgtacctgctgggcatgctggtggccagcgtcctcgccagcttcaacgacgagaagaagctggctagcaacgagatcgcccatctgcccaacctgaacgaaacccagcggagcgccttcctgagcagcatcaacgatgaccccagcaacagcaccaacctgctggccgaggccaagaacctgagcgacgcccaggcccccaagctggaagtgctgtttcaggggcctggcgccaagttcgtggccgcctggacactgaaagccgccgctggcgacgaggtggacggctctcaccaccaccatcaccacagcgcctggtcccacccccagttcgagaagtgatga

SEQ ID NO: 47 - protein, 122 aa, Artificial Sequence
  synthetic construct
MGSLQPLATLYLLGMLVASVLASFNDEKKLASNEIAHLPNLNETQRSAFLSSINDDPSNSTNLLAEAKNLSDAQAPKLEVLFQGPGAKFVAAWTLKAAAGDEVDGSHHHHHHSAWSHPQFEK

SEQ ID NO: 48 - protein, 55 aa, Artificial Sequence
  synthetic construct
SFNDEKKLASNEIAHLPNLNETQRSAFLSSINDDPSNSTNLLAEAKNLSDAQAPK

SEQ ID NO: 49 - DNA, 372 bp, Artificial Sequence
  synthetic construct
atgggcagcctgcagcctctggccaccctgtacctgctgggcatgctggtggccagcgtcctcgccagcttcaacgacgagaagaagctggctagcaacgagatcgcccatctgcccaacctgaacgaaacccagcggagcgccttcctgagcagcatcaacgatgaccccagcaacagcaccaacctgctggccgaggccaagaacctgagcgacgcccagaacgccagcctggaagtgctgtttcaggggcctggcgccaagttcgtggccgcctggacactgaaagccgccgctggcgacgaggtggacggctctcaccaccaccatcaccacagcgcctggtcccacccccagttcgagaagtgatga

SEQ ID NO: 50 - protein, 122 aa, Artificial Sequence
  synthetic construct
MGSLQPLATLYLLGMLVASVLASFNDEKKLASNEIAHLPNLNETQRSAFLSSINDDPSNSTNLLAEAKNLSDAQNASLEVLFQGPGAKFVAAWTLKAAAGDEVDGSHHHHHHSAWSHPQFEK

SEQ ID NO: 51 - protein, 55 aa, Artificial Sequence
  synthetic construct
SFNDEKKLASNEIAHLPNLNETQRSAFLSSINDDPSNSTNLLAEAKNLSDAQNAS

SEQ ID NO: 52 - DNA, 372 bp, Artificial Sequence
  synthetic construct
atgggcagcctgcagcctctggccaccctgtacctgctgggcatgctggtggccagcgtcctcgccagcttcaacgacgagaagaagctggctagcaacgagatcgcccatctgcccaacctgaacgaaacccagcggagcgccttcctgagcagcatcaacgatgaccccagcaacagcaccaacctgctggccaacgccaccaacctgagcgacgcccagaacgccagcctggaagtgctgtttcaggggcctggcgccaagttcgtggccgcctggacactgaaagccgccgctggcgacgaggtggacggctctcaccaccaccatcaccacagcgcctggtcccacccccagttcgagaagtgatga

SEQ ID NO: 53 - protein, 122 aa, Artificial Sequence
  synthetic construct
MGSLQPLATLYLLGMLVASVLASFNDEKKLASNEIAHLPNLNETQRSAFLSSINDDPSNSTNLLANATNLSDAQNASLEVLFQGPGAKFVAAWTLKAAAGDEVDGSHHHHHHSAWSHPQFEK

SEQ ID NO: 54 - protein, 55 aa, Artificial Sequence
  synthetic construct
SFNDEKKLASNEIAHLPNLNETQRSAFLSSINDDPSNSTNLLANATNLSDAQNAS

SEQ ID NO: 55 - protein, 54 aa, Artificial Sequence; identical to SEQ ID NO: 39
  synthetic construct

SEQ ID NO: 56 - protein, 54 aa, Artificial Sequence
  synthetic construct
SFNDEKKLASNEIAHLPNLNETQRSAFLSSINDDPNQSANLLANATKLNDAQAP

SEQ ID NO: 57 - DNA, 372 bp, Artificial Sequence
  synthetic construct
atgggcagcctgcagcctctggccaccctgtacctgctgggcatgctggtggccagcgtcctcgccagcttcaacgacgagaagaagctcgccagcaaccggatcgccaacctgcccaacctgaacgaggaacagcggagcgccttcctgagcaagatcaacgacgaccccagccagagcgccaatctgctggaagaggccctgaagctgaacgacgcccaggcccagaaactggaagtgctgttccaggggccaggcgccaagtttgtggccgcctggacactgaaggccgctgccggcgacgaggtggacggctctcaccaccaccatcaccacagcgcctggtcccacccccagttcgagaagtgatga

SEQ ID NO: 58 - protein, 122 aa, Artificial Sequence
  synthetic construct
MGSLQPLATLYLLGMLVASVLASFNDEKKLASNRIANLPNLNEEQRSAFLSKINDDPSQSANLLEEALKLNDAQAQKLEVLFQGPGAKFVAAWTLKAAAGDEVDGSHHHHHHSAWSHPQFEK

SEQ ID NO: 59 - protein, 55 aa, Artificial Sequence
  synthetic construct
SFNDEKKLASNRIANLPNLNEEQRSAFLSKINDDPSQSANLLEEALKLNDAQAQK

SEQ ID NO: 60 - DNA, 372 bp, Artificial Sequence
  synthetic construct
atgggcagcctgcagcctctggccaccctgtacctgctgggcatgctggtggcctccgtgctggctagcttcaacgacaagaagaagctggcctccaacgagatcgccaacctgcccaacctgaacgaggaacagcggagcgccttcctgagcaagatcaacgacgaccccagcaagagcgaggaactgctggaagaggccctgaagctgaacgacgcccaggccgacaagctggaagtgctgtttcaggggcctggcgccaagttcgtggccgcctggacactgaaagccgccgctggcgacgaggtggacggctctcaccaccaccatcaccacagcgcctggtcccacccccagttcgagaagtgatga

SEQ ID NO: 61 - protein, 122 aa, Artificial Sequence
  synthetic construct
MGSLQPLATLYLLGMLVASVLASFNDKKKLASNEIANLPNLNEEQRSAFLSKINDDPSKSEELLEEALKLNDAQADKLEVLFQGPGAKFVAAWTLKAAAGDEVDGSHHHHHHSAWSHPQFEK

SEQ ID NO: 62 - protein, 55 aa, Artificial Sequence
  synthetic construct
SFNDKKKLASNEIANLPNLNEEQRSAFLSKINDDPSKSEELLEEALKLNDAQADK

SEQ ID NO: 63 - DNA, 372 bp, Artificial Sequence
  synthetic construct
atgggcagcctgcagcctctggccaccctgtacctgctgggcatgctggtggccagcgtgctcgccagcttcaacgacgagaagaagctggcctccaaccggatcgccaacctgcccaacctgaaccaggaacagcggagcgccttcctgagcaagatcaacgacgaccccagccagagcgccaatctgctggaagaggccctgaagctgaacgacaaccaggcccagaaactggaagtgctgttccaggggcctggcgccaagtttgtggccgcctggacactgaaggccgctgccggcgacgaggtggacggctctcaccaccaccatcaccacagcgcctggtcccacccccagttcgagaagtgatga

SEQ ID NO: 64 - protein, 122 aa, Artificial Sequence
  synthetic construct
MGSLQPLATLYLLGMLVASVLASFNDEKKLASNRIANLPNLNQEQRSAFLSKINDDPSQSANLLEEALKLNDNQAQKLEVLFQGPGAKFVAAWTLKAAAGDEVDGSHHHHHHSAWSHPQFEK

SEQ ID NO: 65 - protein, 55 aa, Artificial Sequence
  synthetic construct
SFNDEKKLASNRIANLPNLNQEQRSAFLSKINDDPSQSANLLEEALKLNDNQAQK

SEQ ID NO: 66 - protein, 55 aa, Artificial Sequence; identical to SEQ ID NO: 59
  synthetic construct

SEQ ID NO: 67 - protein, 55 aa, Artificial Sequence
  synthetic construct
SFNDDKKLASNRIANLPNLNEEQRSAFLSKINDDPSQSRNLLEEALKLNDAQAQK

SEQ ID NO: 68 - protein, 55 aa, Artificial Sequence
  synthetic construct
SFNDKKKLASNRIANLPNLNEEQRSAFLSKINDDPSKSEELLEKALKLNDAQAQK

SEQ ID NO: 69 - protein, 55 aa, Artificial Sequence; identical to SEQ ID NO: 62
  synthetic construct

SEQ ID NO: 70 - protein, 55 aa, Artificial Sequence
  synthetic construct
SFNDKKKLASNRIAKLPNLNEKQRSAFLSKINDDPSKSEELLKKALKLNKAQAKK

SEQ ID NO: 71 - protein, 55 aa, Artificial Sequence; identical to SEQ ID NO: 65
  synthetic construct

SEQ ID NO: 72 - protein, 55 aa, Artificial Sequence
  synthetic construct
SFNDDKKLASNRIANLPNLNQEQRSAFLSKINDDPSQSRNLLEEALKLNDNQAQK

SEQ ID NO: 73 - protein, 55 aa, Artificial Sequence
  synthetic construct
SFNDKKKLASNRIANLPNLNQEQRSAFLSKINDDPSKSEELLEKALKLNDNQAQK

SEQ ID NO: 74 - protein, 55 aa, Artificial Sequence
  synthetic construct
SFNDKKKLASNEIANLPNLNQEQRSAFLSKINDDPSKSEELLEEALKLNDNQADK

SEQ ID NO: 75 - protein, 55 aa, Artificial Sequence
  synthetic construct
SFNDKKKLASNRIAKLPNLNEKQRSAFLSNINDDPSKSEELLEKALKLNQAQAQK

SEQ ID NO: 76 - DNA, 372 bp, Artificial Sequence
  synthetic construct
atgggcagcctgcagcctctggccaccctgtacctgctgggcatgctggtggccagcgtgctggctagcttcaacgacgagaagaagctggccagcaacgagatcgcccatctgcccaacctgaacgaggaacagcggagcgccttcctgagcagcatcaacgatgaccccagccagagcgccaacctgctggccgaggccgccaagctgaacgacgcccaggcccccaagctggaagtgctgtttcagggccctggcgccaagttcgtggccgcttggacactgaaagccgccgctggcgacgaggtggacggctctcaccaccaccatcaccacagcgcctggtcccacccccagttcgagaagtgatga

SEQ ID NO: 77 - protein, 122 aa, Artificial Sequence
  synthetic construct
MGSLQPLATLYLLGMLVASVLASFNDEKKLASNEIAHLPNLNEEQRSAFLSSINDDPSQSANLLAEAAKLNDAQAPKLEVLFQGPGAKFVAAWTLKAAAGDEVDGSHHHHHHSAWSHPQFEK

SEQ ID NO: 78 - protein, 55 aa, Artificial Sequence
  synthetic construct
SFNDEKKLASNEIAHLPNLNEEQRSAFLSSINDDPSQSANLLAEAAKLNDAQAPK

SEQ ID NO: 79 - DNA, 372 bp, Artificial Sequence
  synthetic construct
atgggcagcctgcagcctctggccaccctgtacctgctgggcatgctggtggccagcgtgctggctagcttcaacgacgagaagaagctggccagcaacgagatcgcccatctgcccaacctgaacgaggaacagcggagcgccttcctgagcagcatcaacgatgaccccagccagagcgccaacctgctggccgaggccaagaagctgaacgacgccgctgcccccaagctggaagtgctgtttcagggccctggcgccaagttcgtggccgcttggacactgaaagccgccgctggcgacgaggtggacggctctcaccaccaccatcaccacagcgcctggtcccacccccagttcgagaagtgatga

SEQ ID NO: 80 - protein, 122 aa, Artificial Sequence
  synthetic construct
MGSLQPLATLYLLGMLVASVLASFNDEKKLASNEIAHLPNLNEEQRSAFLSSINDDPSQSANLLAEAKKLNDAAAPKLEVLFQGPGAKFVAAWTLKAAAGDEVDGSHHHHHHSAWSHPQFEK

SEQ ID NO: 81 - protein, 55 aa, Artificial Sequence
  synthetic construct
SFNDEKKLASNEIAHLPNLNEEQRSAFLSSINDDPSQSANLLAEAKKLNDAAAPK

SEQ ID NO: 82 - DNA, 372 bp, Artificial Sequence
  synthetic construct
atgggcagcctgcagcctctggccaccctgtacctgctgggcatgctggtggccagcgtgctggctagcttcaacgacgagaagaagctggccagcaacgagatcgcccatctgcccaacctgaacgaggaacagcggagcgccttcctgagcagcatcaacgatgaccccagccagagcgccaacctgctggccgaggccgccaagctgaacgacgccgctgcccccaagctggaagtgctgtttcagggccctggcgccaagttcgtggccgcttggacactgaaagccgccgctggcgacgaggtggacggctctcaccaccaccatcaccacagcgcctggtcccacccccagttcgagaagtgatga

SEQ ID NO: 83 - protein, 122 aa, Artificial Sequence
  synthetic construct
MGSLQPLATLYLLGMLVASVLASFNDEKKLASNEIAHLPNLNEEQRSAFLSSINDDPSQSANLLAEAAKLNDAAAPKLEVLFQGPGAKFVAAWTLKAAAGDEVDGSHHHHHHSAWSHPQFEK

SEQ ID NO: 84 - protein, 55 aa, Artificial Sequence
  synthetic construct
SFNDEKKLASNEIAHLPNLNEEQRSAFLSSINDDPSQSANLLAEAAKLNDAAAPK

SEQ ID NO: 85 - DNA, 372 bp, Artificial Sequence
  synthetic construct
atgggcagcctgcagcctctggccaccctgtacctgctgggcatgctggtggccagcgtgctggctagcttcaacgacgagaagaagctggccagcaacgagctggcccatctgcccaacctgaacgaggaacagcggagcgccgctctgagcagcatcaacgatgaccccagccagagcgccaacctgctggccgaggccaagaagctgaacgacgcccaggcccccaagctggaagtgctgtttcagggccctggcgccaagttcgtggccgcttggacactgaaagccgccgctggcgacgaggtggacggctctcaccaccaccatcaccacagcgcctggtcccacccccagttcgagaagtgatga

SEQ ID NO: 86 - protein, 122 aa, Artificial Sequence
  synthetic construct
MGSLQPLATLYLLGMLVASVLASFNDEKKLASNELAHLPNLNEEQRSAALSSINDDPSQSANLLAEAKKLNDAQAPKLEVLFQGPGAKFVAAWTLKAAAGDEVDGSHHHHHHSAWSHPQFEK

SEQ ID NO: 87 - protein, 55 aa, Artificial Sequence
  synthetic construct
SFNDEKKLASNELAHLPNLNEEQRSAALSSINDDPSQSANLLAEAKKLNDAQAPK

SEQ ID NO: 88 - DNA, 372 bp, Artificial Sequence
  synthetic construct
atgggcagcctgcagcctctggccaccctgtacctgctgggcatgctggtggccagcgtgctggctagcttcaacgacgagaagaagctggccagcaacgagatcgcccatctgcccaacctgaacgaggaacagcggagcgccttcctgagcagcatcaacgatgaccccagccagagcgccaacatcgtggccgaggccaagaagctgaacgacgcccaggcccccaagctggaagtgctgtttcagggccctggcgccaagttcgtggccgcttggacactgaaagccgccgctggcgacgaggtggacggctctcaccaccaccatcaccacagcgcctggtcccacccccagttcgagaagtgatga

SEQ ID NO: 89 - protein, 122 aa, Artificial Sequence
  synthetic construct
MGSLQPLATLYLLGMLVASVLASFNDEKKLASNEIAHLPNLNEEQRSAFLSSINDDPSQSANIVAEAKKLNDAQAPKLEVLFQGPGAKFVAAWTLKAAAGDEVDGSHHHHHHSAWSHPQFEK

SEQ ID NO: 90 - protein, 55 aa, Artificial Sequence
  synthetic construct
SFNDEKKLASNEIAHLPNLNEEQRSAFLSSINDDPSQSANIVAEAKKLNDAQAPK

SEQ ID NO: 91 - DNA, 372 bp, Artificial Sequence
  synthetic construct
atgggcagcctgcagcctctggccaccctgtacctgctgggcatgctggtggccagcgtgctggctagcttcaacgacgagaagaagctggccagcaacgagatcgcccatctgcccaacctgaacgaggaacagcggagcgccttcctgagcagcatcaacgatgaccccagccagagcgccaacatcgccgccgaggccaagaagctgaacgacgcccaggcccccaagctggaagtgctgtttcagggccctggcgccaagttcgtggccgcttggacactgaaagccgccgctggcgacgaggtggacggctctcaccaccaccatcaccacagcgcctggtcccacccccagttcgagaagtgatga

SEQ ID NO: 92 - protein, 122 aa, Artificial Sequence
  synthetic construct
MGSLQPLATLYLLGMLVASVLASFNDEKKLASNEIAHLPNLNEEQRSAFLSSINDDPSQSANIAAEAKKLNDAQAPKLEVLFQGPGAKFVAAWTLKAAAGDEVDGSHHHHHHSAWSHPQFEK

SEQ ID NO: 93 - protein, 55 aa, Artificial Sequence
  synthetic construct
SFNDEKKLASNEIAHLPNLNEEQRSAFLSSINDDPSQSANIAAEAKKLNDAQAPK

SEQ ID NO: 94 - DNA, 372 bp, Artificial Sequence
  synthetic construct
atgggcagcctgcagcctctggccaccctgtacctgctgggcatgctggtggccagcgtgctggctagcttcaacgacgagaagaagctggccagcaacgaggccgcccatctgcccaacctgaacgaggaacagcggagcgccttcctgagcagcatcaacgatgaccccagccagagcgccaacctggccgccgaggccaagaagctgaacgacgcccaggcccccaagctggaagtgctgtttcagggccctggcgccaagttcgtggccgcttggacactgaaagccgccgctggcgacgaggtggacggctctcaccaccaccatcaccacagcgcctggtcccacccccagttcgagaagtgatga

SEQ ID NO: 95 - protein, 122 aa, Artificial Sequence
  synthetic construct
MGSLQPLATLYLLGMLVASVLASFNDEKKLASNEAAHLPNLNEEQRSAFLSSINDDPSQSANLAAEAKKLNDAQAPKLEVLFQGPGAKFVAAWTLKAAAGDEVDGSHHHHHHSAWSHPQFEK

SEQ ID NO: 96 - protein, 55 aa, Artificial Sequence
  synthetic construct
SFNDEKKLASNEAAHLPNLNEEQRSAFLSSINDDPSQSANLAAEAKKLNDAQAPK

SEQ ID NO: 97 - DNA, 372 bp, Artificial Sequence
  synthetic construct
atgggcagcctgcagcctctggccaccctgtacctgctgggcatgctggtggccagcgtgctggctagcttcaacgacgagaagaagctggccagcaacgagatcgcccatctgcccaacgccaacgaggaacagcggagcgccttcctgagcagcatcaacgatgaccccagccagagcgccaacctgctggccgaggccaagaagctgaacgacgcccaggcccccaagctggaagtgctgtttcagggccctggcgccaagttcgtggccgcttggacactgaaagccgccgctggcgacgaggtggacggctctcaccaccaccatcaccacagcgcctggtcccacccccagttcgagaagtgatga

SEQ ID NO: 98 - protein, 122 aa, Artificial Sequence
  synthetic construct
MGSLQPLATLYLLGMLVASVLASFNDEKKLASNEIAHLPNANEEQRSAFLSSINDDPSQSANLLAEAKKLNDAQAPKLEVLFQGPGAKFVAAWTLKAAAGDEVDGSHHHHHHSAWSHPQFEK

SEQ ID NO: 99 - protein, 55 aa, Artificial Sequence
  synthetic construct
SFNDEKKLASNEIAHLPNANEEQRSAFLSSINDDPSQSANLLAEAKKLNDAQAPK

SEQ ID NO: 100 - DNA, 372 bp, Artificial Sequence
  synthetic construct
atgggcagcctgcagcctctggccaccctgtacctgctgggcatgctggtggccagcgtgctggctagcttcaacgacgagaagaagctggccagcaacgagatcgcccatctgcccaacgccaacgaggaacagcggagcgccttcctgagcagcatcaacgatgaccccagccagagcgccaacatcctggccgaggccaagaagctgaacgacgcccaggcccccaagctggaagtgctgtttcagggccctggcgccaagttcgtggccgcttggacactgaaagccgccgctggcgacgaggtggacggctctcaccaccaccatcaccacagcgcctggtcccacccccagttcgagaagtgatga

SEQ ID NO: 101 - protein, 122 aa, Artificial Sequence
  synthetic construct
MGSLQPLATLYLLGMLVASVLASFNDEKKLASNEIAHLPNANEEQRSAFLSSINDDPSQSANILAEAKKLNDAQAPKLEVLFQGPGAKFVAAWTLKAAAGDEVDGSHHHHHHSAWSHPQFEK

SEQ ID NO: 102 - protein, 55 aa, Artificial Sequence
  synthetic construct
SFNDEKKLASNEIAHLPNANEEQRSAFLSSINDDPSQSANILAEAKKLNDAQAPK

SEQ ID NO: 103 - DNA, 372 bp, Artificial Sequence
  synthetic construct
atgggcagcctgcagcctctggccaccctgtacctgctgggcatgctggtggccagcgtgctggctagcttcaacgacgagaagaagctggccagcaacgaggccgcccatctgcccaacctgaacgaggaacagcggagcgccttcctgagcagcatcaacgatgaccccagccagagcgccaacctgctggccgaggccaagaagctgaacgacgcccaggcccccaagctggaagtgctgtttcagggccctggcgccaagttcgtggccgcttggacactgaaagccgccgctggcgacgaggtggacggctctcaccaccaccatcaccacagcgcctggtcccacccccagttcgagaagtgatga

SEQ ID NO: 104 - protein, 122 aa, Artificial Sequence
  synthetic construct
MGSLQPLATLYLLGMLVASVLASFNDEKKLASNEAAHLPNLNEEQRSAFLSSINDDPSQSANLLAEAKKLNDAQAPKLEVLFQGPGAKFVAAWTLKAAAGDEVDGSHHHHHHSAWSHPQFEK

SEQ ID NO: 105 - protein, 55 aa, Artificial Sequence
  synthetic construct
SFNDEKKLASNEAAHLPNLNEEQRSAFLSSINDDPSQSANLLAEAKKLNDAQAPK

SEQ ID NO: 106 - DNA, 372 bp, Artificial Sequence
  synthetic construct
atgggcagcctgcagcctctggccaccctgtacctgctgggcatgctggtggccagcgtgctggctagcttcaacgacgagaagaagctggccagcaacgagatcgcccatgcccccaacctgaacgaggaacagcggagcgccttcctgagcagcatcaacgatgaccccagccagagcgccaacctgctggccgaggccaagaagctgaacgacgcccaggcccccaagctggaagtgctgtttcagggccctggcgccaagttcgtggccgcttggacactgaaagccgccgctggcgacgaggtggacggctctcaccaccaccatcaccacagcgcctggtcccacccccagttcgagaagtgatga

SEQ ID NO: 107 - protein, 122 aa, Artificial Sequence
  synthetic construct
MGSLQPLATLYLLGMLVASVLASFNDEKKLASNEIAHAPNLNEEQRSAFLSSINDDPSQSANLLAEAKKLNDAQAPKLEVLFQGPGAKFVAAWTLKAAAGDEVDGSHHHHHHSAWSHPQFEK

SEQ ID NO: 108 - protein, 55 aa, Artificial Sequence
  synthetic construct
SFNDEKKLASNEIAHAPNLNEEQRSAFLSSINDDPSQSANLLAEAKKLNDAQAPK

SEQ ID NO: 109 - DNA, 372 bp, Artificial Sequence
  synthetic construct
atgggcagcctgcagcctctggccaccctgtacctgctgggcatgctggtggccagcgtgctggctagcttcaacgacgagaagaagctggccagcaacgagatcgcccatctgcccaacctgaacgaggaacagcggagcgccgctctgagcagcatcaacgatgaccccagccagagcgccaacctgctggccgaggccaagaagctgaacgacgcccaggcccccaagctggaagtgctgtttcagggccctggcgccaagttcgtggccgcttggacactgaaagccgccgctggcgacgaggtggacggctctcaccaccaccatcaccacagcgcctggtcccacccccagttcgagaagtgatga

SEQ ID NO: 110 - protein, 122 aa, Artificial Sequence
  synthetic construct
MGSLQPLATLYLLGMLVASVLASFNDEKKLASNEIAHLPNLNEEQRSAALSSINDDPSQSANLLAEAKKLNDAQAPKLEVLFQGPGAKFVAAWTLKAAAGDEVDGSHHHHHHSAWSHPQFEK

SEQ ID NO: 111 - protein, 55 aa, Artificial Sequence
  synthetic construct
SFNDEKKLASNEIAHLPNLNEEQRSAALSSINDDPSQSANLLAEAKKLNDAQAPK

SEQ ID NO: 112 - DNA, 372 bp, Artificial Sequence
  synthetic construct
atgggcagcctgcagcctctggccaccctgtacctgctgggcatgctggtggccagcgtgctggctagcttcaacgacgagaagaagctggccagcaacgagatcgcccatctgcccaacctgaacgaggaacagcggagcgccttcctgagcagcatcaacgatgaccccagccagagcgccaacgccctggccgaggccaagaagctgaacgacgcccaggcccccaagctggaagtgctgtttcagggccctggcgccaagttcgtggccgcttggacactgaaagccgccgctggcgacgaggtggacggctctcaccaccaccatcaccacagcgcctggtcccacccccagttcgagaagtgatga

SEQ ID NO: 113 - protein, 122 aa, Artificial Sequence
  synthetic construct
MGSLQPLATLYLLGMLVASVLASFNDEKKLASNEIAHLPNLNEEQRSAFLSSINDDPSQSANALAEAKKLNDAQAPKLEVLFQGPGAKFVAAWTLKAAAGDEVDGSHHHHHHSAWSHPQFEK

SEQ ID NO: 114 - protein, 55 aa, Artificial Sequence
  synthetic construct
SFNDEKKLASNEIAHLPNLNEEQRSAFLSSINDDPSQSANALAEAKKLNDAQAPK

SEQ ID NO: 115 - DNA, 372 bp, Artificial Sequence
  synthetic construct
atgggcagcctgcagcctctggccaccctgtacctgctgggcatgctggtggccagcgtgctggctagcttcaacgacgagaagaagctggccagcaacgagatcgcccatctgcccaacctgaacgaggaacagcggagcgccttcctgagcagcatcaacgatgaccccagccagagcgccaacctggccgccgaggccaagaagctgaacgacgcccaggcccccaagctggaagtgctgtttcagggccctggcgccaagttcgtggccgcttggacactgaaagccgccgctggcgacgaggtggacggctctcaccaccaccatcaccacagcgcctggtcccacccccagttcgagaagtgatga

SEQ ID NO: 116 - protein, 122 aa, Artificial Sequence
  synthetic construct
MGSLQPLATLYLLGMLVASVLASFNDEKKLASNEIAHLPNLNEEQRSAFLSSINDDPSQSANLAAEAKKLNDAQAPKLEVLFQGPGAKFVAAWTLKAAAGDEVDGSHHHHHHSAWSHPQFEK

SEQ ID NO: 117 - protein, 55 aa, Artificial Sequence
  synthetic construct
SFNDEKKLASNEIAHLPNLNEEQRSAFLSSINDDPSQSANLAAEAKKLNDAQAPK

SEQ ID NO: 118 - DNA, 372 bp, Artificial Sequence
  synthetic construct
atgggcagcctgcagcctctggctaccctgtacctgctgggcatgctggtggctagcgtgctcgccagcttcaacgacgagaagaagctgggcagcaacgagatcgcccatctgcccaacctgaacgaggaacagcggagcgccttcctgagcggcatcaacgacgaccccagccagagcgccaacctgctggccgaggccaagaagctgaacgacgcccaggcccccaagctggaagtgctgtttcaggggcctggcgccaagttcgtggccgcctggacactgaaagccgccgctggcgacgaggtggacggctctcaccaccaccatcaccacagcgcctggtcccacccccagttcgagaagtgatga

SEQ ID NO: 119 - protein, 122 aa, Artificial Sequence
  synthetic construct
MGSLQPLATLYLLGMLVASVLASFNDEKKLGSNEIAHLPNLNEEQRSAFLSGINDDPSQSANLLAEAKKLNDAQAPKLEVLFQGPGAKFVAAWTLKAAAGDEVDGSHHHHHHSAWSHPQFEK

SEQ ID NO: 120 - protein, 55 aa, Artificial Sequence
  synthetic construct
SFNDEKKLGSNEIAHLPNLNEEQRSAFLSGINDDPSQSANLLAEAKKLNDAQAPK

SEQ ID NO: 121 - DNA, 372 bp, Artificial Sequence; identical to SEQ ID NO: 118
  synthetic construct

SEQ ID NO: 122 - protein, 122 aa, Artificial Sequence
  synthetic construct
MGSLQPLATLYLLGMLVASVLASFNDGKKLGSNEIAHLPNLNEEQRSGFLSGINDDPSQSANLLAEAKKLNDAQAPKLEVLFQGPGAKFVAAWTLKAAAGDEVDGSHHHHHHSAWSHPQFEK

SEQ ID NO: 123 - protein, 55 aa, Artificial Sequence
  synthetic construct
SFNDGKKLGSNEIAHLPNLNEEQRSGFLSGINDDPSQSANLLAEAKKLNDAQAPK

SEQ ID NO: 124 - DNA, 372 bp, Artificial Sequence
  synthetic construct
atgggcagcctgcagcctctggccaccctgtacctgctgggcatgctggtggccagcgtcctcgccagcttcaacgacgagaagaagctggctagcaacgagatcgcccatctgcccaacctgaacgaggaacagcggagcgccttcctgagcagcatcaacgatgaccccaagaagtccaagaagctgctggccgaggccaagaaactgaacaagaagcaggcccccaagctggaagtgctgttccaggggcctggcgccaagtttgtggccgcctggacactgaaggccgctgccggcgacgaggtggacggctctcaccaccaccatcaccacagcgcctggtcccacccccagttcgagaagtgatga

SEQ ID NO: 125 - protein, 122 aa, Artificial Sequence
  synthetic construct
MGSLQPLATLYLLGMLVASVLASFNDEKKLASNEIAHLPNLNEEQRSAFLSSINDDPKKSKKLLAEAKKLNKKQAPKLEVLFQGPGAKFVAAWTLKAAAGDEVDGSHHHHHHSAWSHPQFEK

SEQ ID NO: 126 - protein, 55 aa, Artificial Sequence
  synthetic construct
SFNDEKKLASNEIAHLPNLNEEQRSAFLSSINDDPKKSKKLLAEAKKLNKKQAPK

SEQ ID NO: 127 - DNA, 372 bp, Artificial Sequence
  synthetic construct
atgggcagcctgcagcctctggccaccctgtacctgctgggcatgctggtggccagcgtgctcgccagcttcaacgacaagaagaagctggctagcaacaagatcgcccatctgcccaacctgaacgaggaacagcggagcaagttcctgagcaagatcaacgataagcccaagaagtccaagaagctgctgaagaaggccaagaaactgaacaagaagcaggcccccaagctggaagtgctgttccaggggcctggcgccaagtttgtggccgcctggacactgaaggccgctgccggcgacgaggtggacggctctcaccaccaccatcaccacagcgcctggtcccacccccagttcgagaagtgatga

SEQ ID NO: 128 - protein, 122 aa, Artificial Sequence
  synthetic construct
MGSLQPLATLYLLGMLVASVLASFNDKKKLASNKIAHLPNLNEEQRSKFLSKINDKPKKSKKLLKKAKKLNKKQAPKLEVLFQGPGAKFVAAWTLKAAAGDEVDGSHHHHHHSAWSHPQFEK

SEQ ID NO: 129 - protein, 55 aa, Artificial Sequence
  synthetic construct
SFNDKKKLASNKIAHLPNLNEEQRSKFLSKINDKPKKSKKLLKKAKKLNKKQAPK

SEQ ID NO: 130 - DNA, 372 bp, Artificial Sequence
  synthetic construct
atgggcagcctgcagcctctggccaccctgtacctgctgggcatgctggtggccagcgtcctggccagcttcaacgacgagaagaagctggctagcaacgagatcgcccatctgcccaacctgaacgaggaacagcggagcgccttcctgagcagcatcaacgatgaccccgaggaaagcgaggacctgctggccgaggccaaagagctgaacgacgagcaggcccccaagctggaagtgctgtttcaggggcctggcgccaagttcgtggccgcctggacactgaaagccgccgctggcgacgaggtggacggctctcaccaccaccatcaccacagcgcctggtcccacccccagttcgagaagtgatga

SEQ ID NO: 131 - protein, 122 aa, Artificial Sequence
  synthetic construct
MGSLQPLATLYLLGMLVASVLASFNDEKKLASNEIAHLPNLNEEQRSAFLSSINDDPEESEDLLAEAKELNDEQAPKLEVLFQGPGAKFVAAWTLKAAAGDEVDGSHHHHHHSAWSHPQFEK

SEQ ID NO: 132 - protein, 55 aa, Artificial Sequence
  synthetic construct
SFNDEKKLASNEIAHLPNLNEEQRSAFLSSINDDPEESEDLLAEAKELNDEQAPK

SEQ ID NO: 133 - DNA, 372 bp, Artificial Sequence
  synthetic construct
atgggcagcctgcagcctctggccaccctgtacctgctgggcatgctggtggccagcgtcctcgccagcttcaacgacgagaagaagctggctagcaacgagatcgcccatctgcccaacctgaacgaggaacagcggagcgagttcctgagcgagatcaacgacgaccccgaggaaagcgaggacctgctggaagaggccgaggaactgaacgacgagcaggcccccaagctggaagtgctgtttcaggggcctggcgccaagttcgtggccgcctggacactgaaagccgccgctggcgacgaggtggacggctctcaccaccaccatcaccacagcgcctggtcccacccccagttcgagaagtgatga

SEQ ID NO: 134 - protein, 122 aa, Artificial Sequence
  synthetic construct
MGSLQPLATLYLLGMLVASVLASFNDEKKLASNEIAHLPNLNEEQRSEFLSEINDDPEESEDLLEEAEELNDEQAPKLEVLFQGPGAKFVAAWTLKAAAGDEVDGSHHHHHHSAWSHPQFEK

SEQ ID NO: 135 - protein, 55 aa, Artificial Sequence
  synthetic construct
SFNDEKKLASNEIAHLPNLNEEQRSEFLSEINDDPEESEDLLEEAEELNDEQAPK

SEQ ID NO: 136 - DNA, 768 bp, Artificial Sequence
  synthetic construct
atgggcagcctgcagcctctggccaccctgtacctgctgggcatgctggtggctagcgtgctggccagcttcaacgacgagaagaagctggctagcaacgagatcgcccatctgcccaacctgaacgaggaacagcggagcgccttcctgagcagcatcaacgatgaccccagccagagcgccaacctgctggccgaggccaagaagctgaacgacgcccaggcccccaagggatccggcagcagccagatccggcagaactacgccaccgacgtggaagccgccgtgaacagcctggtgaacctgtatctgcaggccagctacacctacctgagcctgggcttctacttcgaccgggacgacgtggccctggaaggcgtgtcccacttcttcagagagctggccgaggaaaagagagagggctacgagcggctgctgaagatgcagaaccagagaggcggcagagccctgttccaggacatcaagaagcccgccgaggacgagtggggcaagacccctgatgccatgaaggccgccatggctctggaaaagaaactgaaccaggccctgctggacctgcacgccctgggctccgccagaaccgacccccacctgtgcgactttctggaaacccacttcctggacgaggaagtgaagctgatcaagaagatgggcgaccacctgaccaacctgcacagactgggcggacctgaggccggcctgggagagtacctgttcgagcggctgaccctgaagcacgactgatga

SEQ ID NO: 137 - protein, 254 aa, Artificial Sequence
  synthetic construct
MGSLQPLATLYLLGMLVASVLASFNDEKKLASNEIAHLPNLNEEQRSAFLSSINDDPSQSANLLAEAKKLNDAQAPKGSGSSQIRQNYATDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 138 - protein, 232 aa, Artificial Sequence
  synthetic construct
SFNDEKKLASNEIAHLPNLNEEQRSAFLSSINDDPSQSANLLAEAKKLNDAQAPKGSGSSQIRQNYATDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 139 - protein, 249 aa, Artificial Sequence
  synthetic construct
MGSLQPLATLYLLGMLVASVLASFNDEKKLASNEIAHLPNLNEEQRSAFLSSINDDPSQSANLLAEAKKLNDAQAPKSGESQVRQNFKPEMEEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDGS

SEQ ID NO: 140 - protein, 227 aa, Artificial Sequence
  synthetic construct
SFNDEKKLASNEIAHLPNLNEEQRSAFLSSINDDPSQSANLLAEAKKLNDAQAPKSGESQVRQNFKPEMEEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDGS

SEQ ID NO: 141 - protein, 262 aa, Artificial Sequence
  synthetic construct
MGSLQPLATLYLLGMLVASVLASFNDEKKLASNEIAHLPNLNEEQRSAFLSSINDDPSQSANLLAEAKKLNDAQAPKSGESQVRQNFKPEMEEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDGSAKFVAAWTLKAAA

SEQ ID NO: 142 - protein, 240 aa, Artificial Sequence
  synthetic construct
SFNDEKKLASNEIAHLPNLNEEQRSAFLSSINDDPSQSANLLAEAKKLNDAQAPKSGESQVRQNFKPEMEEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDGSAKFVAAWTLKAAA

SEQ ID NO: 143 - protein, 256 aa, Artificial Sequence
  synthetic construct
MGSLQPLATLYLLGMLVASVLASFNDEKKLASNEIAHLPNLNEEQRSAFLSSINDDPSQSANLLAEAKKLNDAQAPKGGGSGGGSGESQVRQNFKPEMEEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDGS

SEQ ID NO: 144 - protein, 234 aa, Artificial Sequence
  synthetic construct
SFNDEKKLASNEIAHLPNLNEEQRSAFLSSINDDPSQSANLLAEAKKLNDAQAPKGGGSGGGSGESQVRQNFKPEMEEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDGS

SEQ ID NO: 145 - protein, 269 aa, Artificial Sequence
  synthetic construct
MGSLQPLATLYLLGMLVASVLASFNDEKKLASNEIAHLPNLNEEQRSAFLSSINDDPSQSANLLAEAKKLNDAQAPKGGGSGGGSGESQVRQNFKPEMEEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDGSAKFVAAWTLKAAA

SEQ ID NO: 146 - protein, 247 aa, Artificial Sequence
  synthetic construct
SFNDEKKLASNEIAHLPNLNEEQRSAFLSSINDDPSQSANLLAEAKKLNDAQAPKGGGSGGGSGESQVRQNFKPEMEEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDGSAKFVAAWTLKAAA

SEQ ID NO: 147 - DNA, 372 bp, Artificial Sequence
  synthetic construct
atgggcagcctgcagcctctggccacactgtacctgctgggcatgctggtggcctctgtgctggccagcttcaacgacgagaagaagctcgctagcaacgagatccagcatctgcccaacctgaacgaggaacagcggagcgccttcatcagcagcctgaacgatgaccccagccagagcgccaatctgctggccgaggccaagaagctgaacgacgcccaggcccccaagctggaagtgctgttccagggaccaggcgctaaatttgtggccgcatggacactcaaagccgccgctggcgacgaagtggatggcagccaccaccaccatcaccacagcgcctggtcccacccccagttcgagaagtgatga

SEQ ID NO: 148 - protein, 122 aa, Artificial Sequence
  synthetic construct
MGSLQPLATLYLLGMLVASVLASFNDEKKLASNEIQHLPNLNEEQRSAFISSLNDDPSQSANLLAEAKKLNDAQAPKLEVLFQGPGAKFVAAWTLKAAAGDEVDGSHHHHHHSAWSHPQFEK

SEQ ID NO: 149 - protein, 55 aa, Artificial Sequence
  synthetic construct
SFNDEKKLASNEIQHLPNLNEEQRSAFISSLNDDPSQSANLLAEAKKLNDAQAPK

SEQ ID NO: 150 - DNA, 1905 bp, Artificial Sequence
  synthetic construct
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatcagcggccaccaccaccaccaccaccaccacgactacgacatcccctcctccgagaacctgtacttccagggatctgctagctggagccacccgcagttcgaaaaaagcggccaccaccaccaccaccaccaccacgactacgacatcccctcctccctggaagttctgttccaggggcccggatccgatgaagttgatggtagcccgaatagccgtgttgatgaactgggttttaatgaagccgaacgccagaaaattctggatagcaatagcagcctgatgcgtaatgcaaatgaagtgcgcgataaatttattcagaattatgcgaccagcctgaaagatagcaatgatccgcaggattttctgcgtcgtgttcaagaactgcgtattaatatgcagaaaaattttattagctttgatgcctattataactatctgaataatctggttctggcaagcgcaaatcgttgtagccaggaactgagctttgcaaacgataccattaaaaataacgataccaaaaaactgtttagcaatgaaattgccgataattttaataactttaccgcagatgaagttgcacgtattagcgatctggttgcaagctatctgccgcgtgaatatctgccgccgtttattgatggtaatatgatgggtgtggcctttcagattctgggcattgatgattttggcaaaaaactgaatgaaattgtgcaggatattggcaccaaatattgatga

SEQ ID NO: 151 - protein, 633 aa, Artificial Sequence
  synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISGHHHHHHHHDYDIPSSENLYFQGSASWSHPQFEKSGHHHHHHHHDYDIPSSLEVLFQGPGSDEVDGSPNSRVDELGFNEAERQKILDSNSSLMRNANEVRDKFIQNYATSLKDSNDPQDFLRRVQELRINMQKNFISFDAYYNYLNNLVLASANRCSQELSFANDTIKNNDTKKLFSNEIADNFNNFTADEVARISDLVASYLPREYLPPFIDGNMMGVAFQILGIDDFGKKLNEIVQDIGTKY

SEQ ID NO: 152 - protein, 179 aa, Artificial Sequence
  synthetic construct
GSPNSRVDELGFNEAERQKILDSNSSLMRNANEVRDKFIQNYATSLKDSNDPQDFLRRVQELRINMQKNFISFDAYYNYLNNLVLASANRCSQELSFANDTIKNNDTKKLFSNEIADNFNNFTADEVARISDLVASYLPREYLPPFIDGNMMGVAFQILGIDDFGKKLNEIVQDIGTKY

SEQ ID NO: 153 - DNA, 1905 bp, Artificial Sequence
  synthetic construct
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatcagcggccaccaccaccaccaccaccaccacgactacgacatcccctcctccgagaacctgtacttccagggatctgctagctggagccacccgcagttcgaaaaaagcggccaccaccaccaccaccaccaccacgactacgacatcccctcctccctggaagttctgttccaggggcccggatccgatgaagttgatggtagcccgaatagccgtgttgatgaactgggttttaatgaagccgaacgccagaaaattctggatagcaatagcagcctgatgcgtaatgcaaatgaagtgcgcgataaatttattcagaattatgcgaccagcctgaaagatagcaatgatccgcaggattttctgcgtcgtgttcaagaactgcgtattaatatgcagaaaaattttattagctttgatgcctactataattatctgaataatctggttctggcaagcgcaaatcgtgcaagccaggaactgagctttattaacgataccattaaaaacaatgataccaaaaaactgtttagcaatgaagcagccgataattttaataactttaccgcagatgaagttgcacgtattagcgatctggttgcaagctatctgcctcgtgaatatctgcctccgtttattgatggtaatatgatgggtgtggcctttcagattctgggcattgatgattttggcaaaaaactgaatgaaattgtgcaggatattggcaccaaatattgatga

SEQ ID NO: 154 - protein, 633 aa, Artificial Sequence
  synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISGHHHHHHHHDYDIPSSENLYFQGSASWSHPQFEKSGHHHHHHHHDYDIPSSLEVLFQGPGSDEVDGSPNSRVDELGFNEAERQKILDSNSSLMRNANEVRDKFIQNYATSLKDSNDPQDFLRRVQELRINMQKNFISFDAYYNYLNNLVLASANRASQELSFINDTIKNNDTKKLFSNEAADNFNNFTADEVARISDLVASYLPREYLPPFIDGNMMGVAFQILGIDDFGKKLNEIVQDIGTKY

SEQ ID NO: 155 - protein, 179 aa, Artificial Sequence
  synthetic construct
GSPNSRVDELGFNEAERQKILDSNSSLMRNANEVRDKFIQNYATSLKDSNDPQDFLRRVQELRINMQKNFISFDAYYNYLNNLVLASANRASQELSFINDTIKNNDTKKLFSNEAADNFNNFTADEVARISDLVASYLPREYLPPFIDGNMMGVAFQILGIDDFGKKLNEIVQDIGTKY

SEQ ID NO: 156 - DNA, 1902 bp, Artificial Sequence
  synthetic construct
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatcagcggccaccaccaccaccaccaccaccacgactacgacatcccctcctccgagaacctgtacttccagggatctgctagctggagccacccgcagttcgaaaaaagcggccaccaccaccaccaccaccaccacgactacgacatcccctcctccctggaagttctgttccaggggcccggatccgatgaagttgatggtagcccgaatagccgtgttgatgaactgggttttaatgaagccgaacgccagaaaattctggatagcaatagcagcctgatgcgtaatgcaaatgaagtgcgcgataaatttattcagaattatgcgaccagcctgaaagatagcaatgatccgcaggattttctgcgtcgtgttcaagaactgcgtattaatatgcagaaaaattttattagctttgatgcctactataattatctgaataatctggttctggcaagcgcaaatcgtgcaagccaggaactgagctttgcaaacgataccattaaaaacaatgataccaaaaaactgtttagcaatgaaattgccgataattttaataactttaccgcagatgaagttgcacgtattagcgatctggttgcaagctatctgcctcgtgaatatctgcctccgtttattgatggtaatatgatgggtgtggcctttcagattctgggcattgatgattttggcaaaaaactgaatgaaattgtgcaggatattggcaccaaatgatga

SEQ ID NO: 157 - protein, 632 aa, Artificial Sequence
  synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISGHHHHHHHHDYDIPSSENLYFQGSASWSHPQFEKSGHHHHHHHHDYDIPSSLEVLFQGPGSDEVDGSPNSRVDELGFNEAERQKILDSNSSLMRNANEVRDKFIQNYATSLKDSNDPQDFLRRVQELRINMQKNFISFDAYYNYLNNLVLASANRASQELSFANDTIKNNDTKKLFSNEIADNFNNFTADEVARISDLVASYLPREYLPPFIDGNMMGVAFQILGIDDFGKKLNEIVQDIGTK

SEQ ID NO: 158 - protein, 178 aa, Artificial Sequence
  synthetic construct
GSPNSRVDELGFNEAERQKILDSNSSLMRNANEVRDKFIQNYATSLKDSNDPQDFLRRVQELRINMQKNFISFDAYYNYLNNLVLASANRASQELSFANDTIKNNDTKKLFSNEIADNFNNFTADEVARISDLVASYLPREYLPPFIDGNMMGVAFQILGIDDFGKKLNEIVQDIGTK

SEQ ID NO: 159 - DNA, 1947 bp, Artificial Sequence
  synthetic construct
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatcagcggccaccaccaccaccaccaccaccacgactacgacatcccctcctccgagaacctgtacttccagggatctgctagctggagccacccgcagttcgaaaaaagcggccaccaccaccaccaccaccaccacgactacgacatcccctcctccctggaagttctgttccaggggcccggatccgatgaagttgatggtgcaaaatttgttgcagcatggaccctgaaagcagcagccggtggtagcccgaatagccgtgttgatgaactgggttttaatgaagcagaacgccagaaaattctggatagcaatagcagcctgatgcgtaatgcaaatgaagtgcgcgataaatttattcagaattatgcgaccagcctgaaagatagcaatgatccgcaggattttctgcgtcgtgttcaagaactgcgtattaacatgcagaaaaattttattagctttgatgcctactataattatctgaataatctggttctggccagcgcaaatcgtgcaagccaagaactgagctttgcaaacgataccattaaaaataacgataccaaaaaactgtttagcaatgaaattgccgataattttaataattttaccgcagatgaagtggcacgtattagcgatctggttgcaagctatctgccgcgtgaatatctgccgccgtttattgatggtaatatgatgggtgtggcctttcagattctgggtattgatgattttggcaaaaaactgaatgaaattgtgcaggatattggcaccaaatgatga

SEQ ID NO: 160 - protein, 647 aa, Artificial Sequence
  synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISGHHHHHHHHDYDIPSSENLYFQGSASWSHPQFEKSGHHHHHHHHDYDIPSSLEVLFQGPGSDEVDGAKFVAAWTLKAAAGGSPNSRVDELGFNEAERQKILDSNSSLMRNANEVRDKFIQNYATSLKDSNDPQDFLRRVQELRINMQKNFISFDAYYNYLNNLVLASANRASQELSFANDTIKNNDTKKLFSNEIADNFNNFTADEVARISDLVASYLPREYLPPFIDGNMMGVAFQILGIDDFGKKLNEIVQDIGTK

SEQ ID NO: 161 - protein, 193 aa, Artificial Sequence
  synthetic construct
GAKFVAAWTLKAAAGGSPNSRVDELGFNEAERQKILDSNSSLMRNANEVRDKFIQNYATSLKDSNDPQDFLRRVQELRINMQKNFISFDAYYNYLNNLVLASANRASQELSFANDTIKNNDTKKLFSNEIADNFNNFTADEVARISDLVASYLPREYLPPFIDGNMMGVAFQILGIDDFGKKLNEIVQDIGTK

SEQ ID NO: 162 - DNA, 1902 bp, Artificial Sequence
  synthetic construct
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatcagcggccaccaccaccaccaccaccaccacgactacgacatcccctcctccgagaacctgtacttccagggatctgctagctggagccacccgcagttcgaaaaaagcggccaccaccaccaccaccaccaccacgactacgacatcccctcctccctggaagttctgttccaggggcccggatccgatgaagttgatggtagcccgaatagccgtgttgatgaactgggttttaatgaagccgaacgccagaaaattctggatagcaatagcagcctgatgcgtaatgcaaatgaagtgcgcgataaatttattcagaattatgcgaccagcctgaaagatagcaatgatccgcaggattttctgcgtcgtgttcaagaactgcgtattaatatgcagaaaaattttattagctttgatgcctactataattatctgaataatctggttctggcaagcgcaaatcgtgcaagccaggaactgagctttattaacgataccattaaaaacaatgataccaaaaaactgtttagcaatgaagcagccgataattttaataactttaccgcagatgaagttgcacgtattagcgatctggttgcaagctatctgcctcgtgaatatctgcctccgtttattgatggtaatatgatgggtgtggcctttcagattctgggcattgatgattttggcaaaaaactgaatgaaattgtgcaggatattggcaccaaatgatga

SEQ ID NO: 163 - protein, 632 aa, Artificial Sequence
  synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISGHHHHHHHHDYDIPSSENLYFQGSASWSHPQFEKSGHHHHHHHHDYDIPSSLEVLFQGPGSDEVDGSPNSRVDELGFNEAERQKILDSNSSLMRNANEVRDKFIQNYATSLKDSNDPQDFLRRVQELRINMQKNFISFDAYYNYLNNLVLASANRASQELSFINDTIKNNDTKKLFSNEAADNFNNFTADEVARISDLVASYLPREYLPPFIDGNMMGVAFQILGIDDFGKKLNEIVQDIGTK

SEQ ID NO: 164 - protein, 178 aa, Artificial Sequence
  synthetic construct
GSPNSRVDELGFNEAERQKILDSNSSLMRNANEVRDKFIQNYATSLKDSNDPQDFLRRVQELRINMQKNFISFDAYYNYLNNLVLASANRASQELSFINDTIKNNDTKKLFSNEAADNFNNFTADEVARISDLVASYLPREYLPPFIDGNMMGVAFQILGIDDFGKKLNEIVQDIGTK

SEQ ID NO: 165 - DNA, 828 bp, Artificial Sequence
  synthetic construct
atgggcagcctgcagcctctggccacactgtacctgctgggcatgctggtggcctctgtgctggccgctcccagaggctacaccacctgggtgaacaccatccagaccaacggcctgctgaacgaggccagccagaacctgttcggcatcctgagcgtggacgccaccagcgaggaaatgaacgccttcctggacgtggtgccaggacaggccggacagaagcagatcctgctggacgccatcgacaagatcgccgacgactgggacaacagacaccccctgcccaatgcccctctggtggcccctcctcagggccctatccccatgaccgcccggtttatcagaggcctgggcgtgccacgggagagacagatggaacccgccttcgaccagttccggcagacctaccggcagtggatcatcgaggccatgagcgagggcatcaaagtgatgatcggcaagcccaaggcccagaacatccggcagggcgccaaagagccctaccccgagttcgtgagccggctgctgagccagatcaacgacgagggccaccccaacgacatcaagaagctgcggagcaacaccctgaccatccagaacgccaacgaggaatgccggaacgccatgagacacctgaggcccagcgacacaggcgccgagaagatgtacgcctgccgggacatcggcctggaagtgctgtttcagggcccaggagcaaaatttgtcgccgcctggaccctgaaagccgccgctggcgacgaagtggatggcagccaccaccaccatcaccacagcgcctggtcccacccccagttcgagaagtgatga

SEQ ID NO: 166 - protein, 274 aa, Artificial Sequence
  synthetic construct
MGSLQPLATLYLLGMLVASVLAAPRGYTTWVNTIQTNGLLNEASQNLFGILSVDATSEEMNAFLDVVPGQAGQKQILLDAIDKIADDWDNRHPLPNAPLVAPPQGPIPMTARFIRGLGVPRERQMEPAFDQFRQTYRQWIIEAMSEGIKVMIGKPKAQNIRQGAKEPYPEFVSRLLSQINDEGHPNDIKKLRSNTLTIQNANEECRNAMRHLRPSDTGAEKMYACRDIGLEVLFQGPGAKFVAAWTLKAAAGDEVDGSHHHHHHSAWSHPQFEK

SEQ ID NO: 167 - protein, 207 aa, Artificial Sequence
  synthetic construct
APRGYTTWVNTIQTNGLLNEASQNLFGILSVDATSEEMNAFLDVVPGQAGQKQILLDAIDKIADDWDNRHPLPNAPLVAPPQGPIPMTARFIRGLGVPRERQMEPAFDQFRQTYRQWIIEAMSEGIKVMIGKPKAQNIRQGAKEPYPEFVSRLLSQINDEGHPNDIKKLRSNTLTIQNANEECRNAMRHLRPSDTGAEKMYACRDIG

SEQ ID NO: 168 - DNA, 444 bp, Artificial Sequence
  synthetic construct
atgggcagcctgcagcctctggccacactgtacctgctgggcatgctggtggcctctgtgctggccggaagccaccaccaccatcaccacagcgcctggtcccacccccagttcgagaagtccggcgacgaggtggacggcgccaaatttgtggccgcctggaccctgaaagccgccgctggcctggaagtgctgttccagggcccaggcaagcccaaggcccagaacatccggcagggcgccaaagagccctaccccgagttcgtgagccggctgctgagccagatcaacgacgagggccaccccaacgacatcaagaagctgcggagcaacaccctgaccatccagaacgccaacgaggaatgccggaacgccatgagacacctgaggcccagcgacacaggcgccgagaagatgtacgcctgccgggacatcggctgatga

SEQ ID NO: 169 - protein, 146 aa, Artificial Sequence
  synthetic construct
MGSLQPLATLYLLGMLVASVLAGSHHHHHHSAWSHPQFEKSGDEVDGAKFVAAWTLKAAAGLEVLFQGPGKPKAQNIRQGAKEPYPEFVSRLLSQINDEGHPNDIKKLRSNTLTIQNANEECRNAMRHLRPSDTGAEKMYACRDIG

SEQ ID NO: 170 - protein, 77 aa, Artificial Sequence
  synthetic construct
GKPKAQNIRQGAKEPYPEFVSRLLSQINDEGHPNDIKKLRSNTLTIQNANEECRNAMRHLRPSDTGAEKMYACRDIG

SEQ ID NO: 171 - DNA, 438 bp, Artificial Sequence
  synthetic construct
atgggcagcctgcagcctctggccacactgtacctgctgggcatgctggtggcctctgtgctggccggcaagcccaaggcccagaacatccggcagggcgccaaagagccctaccccgagttcgtgagccggctgctgagccagatcaacgacgagggccaccccaacgacatcaagaagctgcggagcaacaccctgaccatccagaacgccaacgaggaatgccggaacgccatgagacacctgaggcccagcgacacaggcgccgagaagatgtacgcctgccgggacatcggcctggaagtgctgtttcagggcccaggagcaaaatttgtcgccgcctggaccctgaaagccgccgctggcgacgaagtggatggcagccaccaccaccatcaccacagcgcctggtcccacccccagttcgagaagtgatga

SEQ ID NO: 172 - protein, 144 aa, Artificial Sequence
  synthetic construct
MGSLQPLATLYLLGMLVASVLAGKPKAQNIRQGAKEPYPEFVSRLLSQINDEGHPNDIKKLRSNTLTIQNANEECRNAMRHLRPSDTGAEKMYACRDIGLEVLFQGPGAKFVAAWTLKAAAGDEVDGSHHHHHHSAWSHPQFEK

SEQ ID NO: 173 - protein, 77 aa, Artificial Sequence; identical to SEQ ID NO: 170
  synthetic construct

SEQ ID NO: 174 - protein, 556 aa, respiratory syncytial virus
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNQARSELPRFMNYTLNNAKKTNVTLSKKRKSSFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGIEGRHHHHHH

SEQ ID NO: 175 - protein, 568 aa, respiratory syncytial virus
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 176 - protein, 549 aa, respiratory syncytial virus
GAGMELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARGAGKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 177 - protein, 185 aa, Artificial Sequence
  synthetic construct
HHHHHHGSPNSRVDELGFNEAERQKILDSNSSLMRNANEVRDKFIQNYATSLKDSNDPQDFLRRVQELRINMQKNFISFDAYYNYLNNLVLASANRCSQELSFANDTIKNNDTKKLFSNEIADNFNNFTADEVARISDLVASYLPREYLPPFIDGNMMGVAFQILGIDDFGKKLNEIVQDIGTKY

SEQ ID NO: 178 - protein, 185 aa, Artificial Sequence
  synthetic construct
HHHHHHGSPNSRVDELGFNEAERQKILDSNSSLMRNANEVRDKFIQNYATSLKDSNDPQDFLRRVQELRINMQKNFISFDAYYNYLNNLVLASANRASQELSFINDTIKNNDTKKLFSNEAADNFNNFTADEVARISDLVASYLPREYLPPFIDGNMMGVAFQILGIDDFGKKLNEIVQDIGTKY

SEQ ID NO: 179 - protein, 184 aa, Artificial Sequence
  synthetic construct
HHHHHHGSPNSRVDELGFNEAERQKILDSNSSLMRNANEVRDKFIQNYATSLKDSNDPQDFLRRVQELRINMQKNFISFDAYYNYLNNLVLASANRASQELSFANDTIKNNDTKKLFSNEIADNFNNFTADEVARISDLVASYLPREYLPPFIDGNMMGVAFQILGIDDFGKKLNEIVQDIGTK

SEQ ID NO: 180 - protein, 184 aa, Artificial Sequence
  synthetic construct
HHHHHHGSPNSRVDELGFNEAERQKILDSNSSLMRNANEVRDKFIQNYATSLKDSNDPQDFLRRVQELRINMQKNFISFDAYYNYLNNLVLASANRASQELSFINDTIKNNDTKKLFSNEAADNFNNFTADEVARISDLVASYLPREYLPPFIDGNMMGVAFQILGIDDFGKKLNEIVQDIGTK

SEQ ID NO: 181 - protein, 83 aa, Artificial Sequence
  synthetic construct
HHHHHHGKPKAQNIRQGAKEPYPEFVSRLLSQINDEGHPNDIKKLRSNTLTIQNANEECRNAMRHLRPSDTGAEKMYACRDIG

SEQ ID NO: 182 - protein, 8 aa, Artificial Sequence
  synthetic construct
LEVLFQGP

SEQ ID NO: 183 - protein, 13 aa, Artificial Sequence
  synthetic construct
AKFVAAWTLKAAA

SEQ ID NO: 184 - protein, 4 aa, Artificial Sequence
  synthetic construct
DEVD

SEQ ID NO: 185 - protein, 6 aa, Artificial Sequence
  synthetic construct
HHHHHH

SEQ ID NO: 186 - protein, 8 aa, Artificial Sequence
  synthetic construct
WSHPQFEK

SEQ ID NO: 187 - protein, 103 aa, Artificial Sequence
  synthetic construct
VDNSFNDEKKLASNEIAHLPNLNEEQRSAFLSSINDDPSQSANLLAEAKKLNDAQAPKLEVLFQGPGAKFVAAWTLKAAAGDEVDGSHHHHHHSAWSHPQFEK

SEQ ID NO: 188 - protein, 22 aa, Artificial Sequence
  synthetic construct
MGSLQPLATLYLLGMLVASVLA